SEQUENCE LISTING (13 sequences)

SEQ ID NO: 1 - RNA, 34 nt, Artificial Sequence
  SYNTHETIC CONSTRUCT
agaucggaagagcacacgucugaacuccagucac

SEQ ID NO: 2 - RNA, 35 nt, Artificial Sequence
  SYNTHETIC CONSTRUCT
aagaucggaagagcacacgucugaacuccagucac

SEQ ID NO: 3 - DNA, 35 bp, Artificial Sequence
  SYNTHETIC CONSTRUCT
  misc_feature (35)..(35): n is a, c, g, or t
gtgactggagttcagacgtgtgctcttccgatctn

SEQ ID NO: 4 - DNA, 36 bp, Artificial Sequence
  SYNTHETIC CONSTRUCT
  misc_feature (36)..(36): n is a, c, g, or t
gtgactggagttcagacgtgtgctcttccgatcttn

SEQ ID NO: 5 - DNA, 32 bp, Artificial Sequence
  SYNTHETIC CONSTRUCT
gatcgtcggactgtagaactctgaacgtgtag

SEQ ID NO: 6 - DNA, 55 bp, Artificial Sequence
  SYNTHETIC CONSTRUCT
aatgatacggcgaccaccgagatctacacgttcagagttctacagtccgacgatc

SEQ ID NO: 7 - DNA, 58 bp, Artificial Sequence
  SYNTHETIC CONSTRUCT
caagcagaagacggcatacgagatgtgactggagttcagacgtgtgctcttccgatct

SEQ ID NO: 8 - RNA, 15 nt, Artificial Sequence
  SYNTHETIC CONSTRUCT
  misc_feature (8)..(15): n is a, c, g, or u
uccaucannnnnnnn

SEQ ID NO: 9 - RNA, 35 nt, Artificial Sequence; identical to SEQ ID NO: 2
  SYNTHETIC CONSTRUCT

SEQ ID NO: 10 - DNA, 36 bp, Artificial Sequence; identical to SEQ ID NO: 4
  SYNTHETIC CONSTRUCT
  misc_feature (36)..(36): n is a, c, g, or t

SEQ ID NO: 11 - DNA, 32 bp, Artificial Sequence; identical to SEQ ID NO: 5
  SYNTHETIC CONSTRUCT

SEQ ID NO: 12 - DNA, 55 bp, Artificial Sequence; identical to SEQ ID NO: 6
  SYNTHETIC CONSTRUCT

SEQ ID NO: 13 - DNA, 58 bp, Artificial Sequence; identical to SEQ ID NO: 7
  SYNTHETIC CONSTRUCT

(19) United States
(12) Patent Application Publication
(10) Pub. No.: US 2024/0218448 A1
(43) Pub. Date: Jul. 4, 2024

(54) METHODS AND COMPOSITIONS RELATED TO FULL-LENGTH EXCISED INTRON RNAS (FLEXI RNAS)

(71) Applicant: Board of Regents, The University of Texas System, Austin, TX (US)

(72) Inventors: Alan Lambowitz, Austin, TX (US); Jun Yao, Austin, TX (US); Ching Kai Douglas Wu, Rockville, MD (US); Hengyi XU, Cedar Park, TX (US)

(21) Appl. No.: 17/920,843

(22) PCT Filed: Apr. 23, 2021
(86) PCT No.: PCT/US2021/028826

§ 371 Date: Oct. 24, 2022

Related U.S. Application Data
(60) Provisional application No. 63/014,429, filed on Apr. 23, 2020.

(51) Int. Cl. C12Q 1/6886; C12N 15/10; C12Q 1/6869

(52) U.S. Cl. CPC C12Q 1/6886; C12N 15/1096; C12Q 1/6869; C12Q 2600/106; C12Q 2600/118; C12Q 2600/156; C12Q 2600/158

(57) ABSTRACT

Disclosed herein are methods and compositions related to determining one or more biomarkers in Full-Length Excised Linear Intron RNAs (FLEXI RNAs) and Intron RNA fragments. These FLEXI RNAs and Intron RNA fragments can be indicative of a specific characteristic. trait. disease. disorder or condition. FLEXI RNAs and Intron RNA fragments can be used to establish a predictive bio-marker. a diagnostic biomarker, a prognostic biomarker, or a biomarker that relates to drug interaction, drug response, or to a heritable condition. These biomarkers can then be used to treat. monitor. or inform patients.

### I. CROSS-REFERENCE TO RELATED APPLICATIONS

This application claims benefit of U.S. Provisional Application No. 63/014,429, filed Apr. 23, 2020, incorporated herein by reference in its entirety.

### II. GOVERNMENT SUPPORT

. This invention was made with government support under Grant No. ROI GM037949 and Grant No. R35 GM136216 awarded by the National Institutes of Health. The government has certain rights in the invention.

### III. BACKGROUND

. Introns are segments of an RNA transcript that are flanked by regions of functional importance (exons) and eliminated from transcripts by chemical reactions that precisely excise the intron segment and ligate the flanking exons, a process known as RNA splicing (Chorev et al. 2012). Introns are found in the genes of most organisms and many viruses and can be located in a wide range of genes, including those that encode proteins, ribosomal RNA (rRNA) and transfer RNA (tRNA). A number of different types of introns are known, including eukaryotic spliceosomal introns, tRNA introns, group I introns and group II introns. When proteins are generated from an intron-containing gene, RNA splicing takes place as part of the RNA processing pathway that follows transcription and precedes translation.

Many families of non-coding RNAs (ncRNAs) have been characterized, such as microRNAs (miRNAs), small nucleolar RNAs (snoRNAs), piwi-interacting RNAs (piRNAs), small-interfering RNAs (siRNAs), and various long non-coding RNAs (lncRNAs). In some genes, ncRNAs, such as miRNAs or snoRNAs, are encoded within introns, leading to the hypothesis that some genes may regulate their own expression or that of other genes by hosting regulatory ncRNAs within their introns (Rearick et al. 2011). Some types of introns, such as group I and group II introns, encode functional proteins. In the case of group II introns, these proteins are reverse transcriptases that function in both RNA splicing and mobility (retrotransposition) of the intron to new genomic DNA sites (reviewed in Lambowitz and Zimmerly, 2011). Group II intron-encoded reverse transcriptases have also been found to be useful for biotechnological applications, such as high-throughput RNA sequencing (RNA-seq) (Mohr et al., RNA 2013; Qin et al., RNA 2016; Nottingham et al., RNA 2016). The beneficial properties of group II introns for RNA-seq enable them to accurately reverse transcribe highly structured RNAs, making it possible to obtain full-length end-to-end sequence reads of such RNAs (Katibah et al. Proc. Nat. Acad. Sci., USA, 2014). Group II intron reverse transcriptases from bacterial thermophiles are thermostable and are referred to as thermostable group II intron reverse transcriptases (TGIRT) enzymes, which are sold commercially for RNA-seq applications.

Group II intron reverse transcriptases are members of a larger family of reverse transcriptases known as non-LTR-retroelement reverse transcriptases (sometimes also referred to as non-retroviral reverse transcriptases). Group II intron reverse transciptases are comprised of a reverse transcriptase (RT) domain, which contains seven conserved amino acid sequence blocks (RT1-7), which are found in the fingers an palm regions of retroviral RTs; a thumb domain (sometimes referred to as domain X); a DNA-binding domain, and in some cases, a DNA endonuclease domain (Blocker et al. RNA 2005). The RT and thumb (X) domains of group II intron and other non-LTR-retroelement reverse transcriptases are larger than those of retroviral reverse transcriptases, with the RT domain having a distinctive N-terminal extension (NTE), which can contain a conserved amino acid sequence block denoted (RTO), and two distinctive insertions denoted RT2a and RT3a between the conserved RT sequence blocks (Blocker et al. RNA 2005). Recent structural and biochemical studies have related some of these distinctive structural features to the beneficial properties of group II intron reverse transcriptases for RNA-seq (Stamos et al. Mol. Cell 2017; Lentzsch et al. J. Biol. Chem. 2019).

In eukaryotes, introns in genes encoding proteins and long non-coding RNAs (lncRNAs) are spliced by a complex apparatus known as the spliceosome, which consists of small nuclear RNAs (snRNAs) and approximately 100 proteins (Wilkinson et al. Annu. Rev. Biochem. 2019). Such introns, referred to here as spliceosomal introns, are spliced in two sequential chemical reactions (transesterifications) that produce ligated exons and an excised intron lariat RNA in which the 5′ end of the intron RNA is linked to a branch-point nucleotide, usually an adenosine, near the 3′ end of the intron by a 2′,5′ phosphodiester bond. This linkage leaves a short 3′ tail after the branch point. In most cases, spliceosomal introns are debranched by debranching enzyme DBR1 to produce linear intron RNAs, which are then rapidly degraded by cellular ribonucleases (Chapman and Boeke, Cell 1991).

In a few cases, excised spliceosomal intron RNAs that are not rapidly degraded after excision stable have been identified. Stable intron sequence RNAs (sisRNAs) have been found in the cytoplasm of Xenopus oocytes and Drosophila embryos, as well as human, mouse, chicken, and zebrafish cells (Gardner et al. Genes Dev. 2012; Talhouame and Gall, Proc. Nat. 3′ tail), typically 100-500 nucleotides in length, and often have an unusual cytosine branch-point nucleotide, which may make them resistant to debranching enzyme. Those sisRNAs that have a canonical adenosine branch point may have other structural features that likewise make them resistant to debranching enzyme. Additional examples of stable intron RNAs include a linear sisRNA detected in the cytoplasm of a Drosophila embryo (Pek et al.. J. Cell Biol. 2015) and branched circular intron RNAs that are found in the nucleus and neuronal projections of mammalian cells (Zhang et al. Mol. Cell 2013; Saini et al. eLife, 2019).

Morgan et al. Nature (2019) described 34 excised intron RNAs in the yeast Saccharomyces cerevisiae that are rapidly degraded in log phase cells but are debranched and accumulate as linear RNAs in cells undergoing nutrient starvation or other stresses. In related findings, Parenteau et al. Nature (2019) found that in most cases, yeast cells with deletion of an intron are impaired when nutrients are depleted and suggested that excised intron RNAs that accumulate under these conditions sequester spliceosome components, thereby inhibiting RNA splicing to reduce nutrient consumption and promote cell survival.

Previous examples of structured spliceosomal intron RNAs include mirtrons and agotrons. Mirtrons are pre-miRNA/introns that are excised by RNA splicing, debranched by debranching enzyme (DBR1), and processed by Dicer into mature miRNAs that function in the regulation of gene expression (Berezikov et al. Mol. Cell 2007; Okamura et al. Cell 2007; Ruby et al. Nature 2007), while agotrons are structured intron RNAs that bind Ago2 and function directly to repress target mRNAs in a miRNA-like manner (Hansen, 2018; Hansen et al., 2016). Like mirtrons, agotrons are thought to be excised as lariat RNAs and debranched by debranching enzyme. Based on Northern hybridization experiments and CLIPseq 5′-end sequences, agotrons were hypothesized to function as full-length linear intron RNAs. However, full-length excised intron RNAs corresponding to agotrons or mirtrons pre-miRNAs have not been identified by full-length end-to-end sequence reads using previous RNA-seq methods, likely because the retroviral reverse transcriptases used in these methods are unable to fully reverse transcribe these structured RNAs.

Classification of specific biomarkers can provide a biosignature that can be indicative of a specific characteristic, trait, disease, disorder or condition. What is needed in the art are biomarkers found in full-length excised intron RNAs (FLEXI RNAs).

### IV. SUMMARY

Disclosed are methods and compositions related to determining one or more structured RNAs, making it possible to obtain full-length end-to-end sequence reads of such RNAs (Katibah et al. Proc. Nat. Acad. Sci., USA, 2014). Group II intron reverse transcriptases from bacterial thermophiles are thermostable and are referred to as thermostable group II intron reverse transcriptases (TGIRT) enzymes, which are sold commercially for RNA-seq applications.

Group II intron reverse transcriptases are members of a larger family of reverse transcriptases known as non-LTR-retroelement reverse transcriptases (sometimes also referred to as non-retroviral reverse transcriptases). Group II intron reverse transciptases are comprised of a reverse transcriptase (RT) domain, which contains seven conserved amino acid sequence blocks (RT1-7), which are found in the fingers an palm regions of retroviral RTs; a thumb domain (sometimes referred to as domain X); a DNA-binding domain, and in some cases, a DNA endonuclease domain (Blocker et al. RNA 2005). The RT and thumb (X) domains of group II intron and other non-LTR-retroelement reverse transcriptases are larger than those of retroviral reverse transcriptases, with the RT domain having a distinctive N-terminal extension (NTE), which can contain a conserved amino acid sequence block denoted (RTO), and two distinctive insertions denoted RT2a and RT3a between the conserved RT sequence blocks (Blocker et al. RNA 2005). Recent structural and biochemical studies have related some of these distinctive structural features to the beneficial properties of group II intron reverse transcriptases for RNA-seq (Stamos et al. Mol. Cell 2017; Lentzsch et al. J. Biol. Chem. 2019).

In eukaryotes, introns in genes encoding proteins and long non-coding RNAs (lncRNAs) are spliced by a complex apparatus known as the spliceosome, which consists of small nuclear RNAs (snRNAs) and approximately 100 proteins (Wilkinson et al. Annu. Rev. Biochem. 2019). Such introns, referred to here as spliceosomal introns, are spliced in two sequential chemical reactions (transesterifications) that produce ligated exons and an excised intron lariat RNA in which the 5′ end of the intron RNA is linked to a branch-point nucleotide, usually an adenosine, near the 3′ end of the intron by a 2′,5′ phosphodiester bond. This linkage leaves a short 3′ tail after the branch point. In most cases, spliceosomal introns are debranched by debranching enzyme DBR1 to produce linear intron RNAs, which are then rapidly degraded by cellular ribonucleases (Chapman and Boeke, Cell 1991).

In a few cases, excised spliceosomal intron RNAs that are not rapidly degraded after excision stable have been identified. Stable intron sequence RNAs (sisRNAs) have been found in the cytoplasm of Xenopus oocytes and Drosophila embryos, as well as human, mouse, chicken, and zebrafish cells (Gardner et al. Genes Dev. 2012; Talhouame and Gall, Proc. Nat. 3′ tail), typically 100-500 nucleotides in length, and often have an unusual cytosine branch-point nucleotide, which may make them resistant to debranching enzyme. Those sisRNAs that have a canonical adenosine branch point may have other structural features that likewise make them resistant to debranching enzyme. Additional examples of stable intron RNAs include a linear sisRNA detected in the cytoplasm of a Drosophila embryo (Pek et al.. J. Cell Biol. 2015) and branched circular intron RNAs that are found in the nucleus and neuronal projections of mammalian cells (Zhang et al. Mol. Cell 2013; Saini et al. eLife, 2019).

Morgan et al. Nature (2019) described 34 excised intron RNAs in the yeast Saccharomyces cerevisiae that are rapidly degraded in log phase cells but are debranched and accumulate as linear RNAs in cells undergoing nutrient starvation or other stresses. In related findings, Parenteau et al. Nature (2019) found that in most cases, yeast cells with deletion of an intron are impaired when nutrients are depleted and suggested that excised intron RNAs that accumulate under these conditions sequester spliceosome components, thereby inhibiting RNA splicing to reduce nutrient consumption and promote cell survival.

examples of structured spliceosomal intron RNAs include mirtrons and agotrons. Mirtrons are pre-miRNA/introns that are excised by RNA splicing, debranched by debranching enzyme (DBR1), and processed by Dicer into mature miRNAs that function in the regulation of gene expression (Berezikov et al. Mol. Cell 2007; Okamura et al. Cell 2007; Ruby et al. Nature 2007), while agotrons are structured intron RNAs that bind Ago2 and function directly to repress target mRNAs in a miRNA-like manner (Hansen, 2018; Hansen et al., 2016). Like mirtrons, agotrons are thought to be excised as lariat RNAs and debranched by debranching enzyme. Based on Northern hybridization experiments and CLIPseq 5′-end sequences, agotrons were hypothesized to function as full-length linear intron RNAs. However, full-length excised intron RNAs corresponding to agotrons or mirtrons pre-miRNAs have not been identified by full-length end-to-end sequence reads using previous RNA-seq methods, likely because the retroviral reverse transcriptases used in these methods are unable to fully reverse transcribe these structured RNAs.

Classification of specific biomarkers can provide a biosignature that can be indicative of a specific characteristic, trait, disease, disorder or condition. What is needed in the art are biomarkers found in full-length excised intron RNAs (FLEXI RNAs).

### V. SUMMARY

Disclosed are methods and compositions related to determining one or more RNAs which are less than 300 nucleotides in length, with 5′ and 3′ ends within 3 nucleotides of annotated splice sites), wherein said one or more biomarkers are indicative of a specific characteristic, trait, disease, disorder or condition, the method comprising: a) obtaining FLEXI RNAs from one or more subjects with a specific characteristic, trait, disease, disorder or condition; b) determining the sequence or sequences of the FLEXI RNAs from said one or more subjects; c) comparing the sequence or sequences of said FLEXI RNAs from subjects with a specific characteristic, trait, disease, disorder or condition to sequences of control FLEXI RNAs to determine differences; and d) determining which differences are indicative of a specific characteristic, trait, disease, disorder or condition, thereby identifying biomarkers for said specific characteristic, trait, disease, disorder or condition. Also disclosed are fragments of an Intron RNA

Said FLEXI RNAs can be identified by RNA sequencing, preferably by an RNA-sequencing method that utilizes a non-LTR-retroelement reverse transcriptase to obtain full-length end-to-end sequence reads of FLEXI RNAs. The non-LTR retroelement reverse transcriptase can be a group II intron-encoded reverse transcriptase, for example. Once identified, FLEXI RNAs can be detected and quantitated by a variety of methods, including RT-qPCR, microarrays or other nucleic acid hybridization-based methods, or targeted RNA-seq. FLEXI RNAs found to be useful biomarkers for a specific trait could be incorporated into targeted RNA panels and kits by themselves or together with other RNA or non-RNA analytes for a variety of applications, including those using diagnostic, predictive, or prognostic biomarkers.

The FLEXI RNAs discovered by the methods disclosed herein can be useful in determining gene expression, alternative splicing, or differential stability. The biomarkers disclosed herein can be for a specific disease such as cancer (for example breast cancer), an infectious disease, an autoimmune disease, tissue damage, or a mental disease. The biomarker can be a predictive biomarker, a diagnostic biomarker, a prognostic biomarker, or can relate to drug interaction, drug response, or to a heritable condition. The biomarkers can be used to track disease progression and response to treatment in a subject.

One, or more than one, biomarkers in FLEXI RNAs can be determined using the methods described herein. For example, two or more FLEXI RNA biomarkers can be determined. When at least two biomarkers are present together, they can be indicative of a specific characteristic, trait, disease, disorder or condition. The two biomarkers can be present in the same, or in two or more different, genes.

In determining biomarkers using the methods disclosed herein, control FLEXI RNAs from one or more subjects without the specific characteristic, trait, disease, disorder or condition can be used. The biomarkers disclosed herein can be part of a panel. For example, the panel can include FLEXI RNAs discovered using the methods discussed herein. The panel can also comprise control FLEXI RNAs.

The methods disclosed herein of determining which differences are indicative of a specific characteristic, trait, disease, disorder or condition can be carried out via computer program The FLEXI RNAs disclosed herein can be specific for a cell or tissue type, and can be obtained from a variety of sources, including plasma.

Further disclosed herein is a method of treating or preventing a disease or disorder in a subject, the method comprising: a) obtaining a sample from a subject; b) sequencing all or a portion of one or more Full-Length Excised Intron RNAs (FLEXI RNAs); c) analyzing sequence data from step b); d) determining that the subject has a disease or disorder based on results of step c); and e) treating or preventing the disease or disorder in the subject. After obtaining a sample from the subject, RNA can be isolated. Said FLEXI RNAs can be sequenced or analyzed using RT-qPCR, a microarray or other hybridization-based assay, or targeted RNA-seq. The specific disease can be cancer, an infectious disease, an autoimmune disease, tissue damage, or a mental disease. At least two different biomarkers can be used to determine that the subject has a disease or disorder. Said FLEXI RNAs, and biomarkers thereof, can comprise a panel. The panel can further comprise control FLEXI RNAs. Biomarkers in said FLEXI RNAs from said subject can be compared to a set of control FLEXI RNAs to determine differences in the subject's FLEXI RNAs which are related to a disease or disorder. This method can be done via computer program

Further disclosed herein is a method of treating a subject based on disease prognosis for the subject, the method comprising: a) obtaining a sample from a subject; b) sequencing all or a portion of one or more Full-Length Excised Intron RNAs (FLEXI RNAs); c) analyzing sequence data from step b); d) determining disease prognosis for the subject based on results of step c); and e) treating the disease or disorder in the subject according to said prognosis. After obtaining a sample from the subject, RNA can be isolated. Said FLEXI RNAs can be sequenced or analyzed using RT-qPCR, a microarray or other hybridization-based assay, or targeted RNA-seq. The specific disease can be cancer, an infectious disease, an autoimmune disease, tissue damage, or a mental disease. At least two different biomarkers can be used in the prognosis of the subject. Said FLEXI RNAs, and biomarkers thereof, can comprise a panel. The panel can further comprise control FLEXI RNAs. Biomarkers in said FLEXI RNAs from said subject can be compared to a set of control FLEXI RNAs to determine differences in the subject's FLEXI RNAs which are related to prognosis of a given disease or disorder. This method can be done via computer program.

Disclosed herein is a method of determining potential drug interaction for a subject and treating the subject accordingly, the method comprising: a) obtaining a sample from a subject; b) sequencing all or a portion of one or more Full-Length Excised Intron RNAs (FLEXI RNAs); c) analyzing sequence data from step b) to determine potential drug interactions; and d) administering a drug or drugs based on the results of step c). After obtaining a sample from the subject, RNA can be isolated. Said FLEXI RNAs can be sequenced or analyzed using RT-qPCR, a microarray or other hybridization-based assay, or targeted RNA-seq. At least two different biomarkers can be used to determine potential drug interactions for the subject. Said FLEXI RNAs, and biomarkers thereof, can comprise a panel. The panel can further comprise control FLEXI RNAs. Biomarkers in said FLEXI RNAs from said subject can be compared to a set of control FLEXI RNAs to determine differences in the subject's FLEXI RNAs which are related to potential drug interaction. This method can be done via computer program.

Further disclosed is a method of determining potential response to a drug in a subject and administering a drug based on results thereof, the method comprising: a) obtaining a sample from a subject; b) sequencing all or a portion of one or more Full-Length Excised Intron RNAs (FLEXI RNAs); c) analyzing sequence data from step b) to determine potential response to a drug; and d) administering a drug or drugs based on the results of step c). After obtaining a sample from the subject, RNA can be isolated. Said FLEXI RNAs can be sequenced or analyzed using RT-qPCR, a microarray or other hybridization-based assay, or targeted RNA-seq. At least two different biomarkers can be used to determine potential drug response for the subject. Said FLEXI RNAs, and biomarkers thereof, can comprise a panel. The panel can further comprise control FLEXI RNAs. Biomarkers in said FLEXI RNAs from said subject can be compared to a set of control FLEXI RNAs to determine differences in the subject's FLEXI RNAs which are related to potential drug response. This method can be done via computer program.

Also disclosed herein is a method of tracking disease progression and/or response to treatment in a subject, and treating the subject accordingly, the method comprising: a) obtaining a sample from a subject; b) sequencing all or a portion of one or more Full-Length Excised Intron RNAs (FLEXI RNAs); c) analyzing sequence data from step b) to determine disease progression and/or treatment response; and d) treating the subject based on the results of step c). RNA can be isolated after the sample is obtained. Said FLEXI RNAs can be sequenced or analyzed using RT-qPCR, a microarray or other hybridization-based assay, or targeted RNA-seq. At least two different biomarkers can be used to determine disease progression and/or treatment response of the subject. Said FLEXI RNAs can comprise a panel, which can optionally include control FLEXI RNAs and/or other RNA or non-RNA analytes. Comparing said FLEXI RNAs from said subject to a set of control FLEXI RNAs to determine differences in the subject's FLEXI RNAs which are related to a disease or disorder, can be done via computer program

Disclosed herein is a computer-implemented method for providing an evaluation for display, which evaluation is with respect to identifying one or more variations in one or more FLEXI RNAs that are associated with a specific characteristic, trait, disease, disorder or condition, comprising: a) obtaining sequence data from one or more FLEXI RNAs from subjects with and without a specific characteristic, trait, disease, disorder or condition; b) evaluating FLEXI RNA data from step a) using computer software executed on a computer to determine relevant biomarkers for a specific characteristic, trait, disease, disorder or condition, wherein said evaluation is algorithmically constructed and manipulated to detect patterns; and c) providing said evaluation for display on a computer generated report that identifies said one or more biomarkers in one or more FLEXI RNAs that are indicative of a specific characteristic, trait, disease, disorder or condition. Said FLEXI RNAs can be sequenced by RNA sequencing, such as by using a non-LTR-retroelement reverse transcriptase-based method. A group II intron-encoded reverse transcriptase in an example thereof.

The FLEXI RNAs can be useful in determining gene expression, alternative splicing, or differential stability in the computer-implemented methods disclosed herein. The biomarkers disclosed herein can be for a specific disease such as cancer (such as breast cancer), an infectious disease, an autoimmune disease, tissue damage, or mental disease. The biomarker can be a predictive biomarker, a diagnostic biomarker, a prognostic biomarker, or can relate to drug interaction, drug response, or to a heritable condition. The biomarkers can be used to track disease progression in a subject.

One, or more than one, FLEXI-RNAs can be determined using the computer-implemented methods described herein. For example, two or more FLEXI RNA biomarkers can be determined. When at least two biomarkers are present together, they can be indicative of a specific characteristic, trait, disease, disorder or condition. The two biomarkers can be present in the same, or in two or more different, genes. In determining biomarkers using the computer-implemented methods disclosed herein, control FLEXI RNAs from one or more subjects without the specific characteristic, trait, disease, disorder or condition can be used. The biomarkers disclosed herein for use in a computer-implemented method can be part of a panel. For example, the panel can include FLEXI RNAs discovered using the methods discussed herein. The panel can also comprise control FLEXI RNAs. The FLEXI RNAs disclosed herein for use in computer-implemented methods can be specific for a cell or tissue type, and can be obtained from a variety of sources, including plasma.

Further disclosed herein is a computer-implemented display for displaying the biomarkers identified in the computer-implemented methods disclosed herein.

Also disclosed is an assay comprising a panel of biomarkers, wherein said biomarkers are found in FLEXI RNAs, wherein said biomarkers are indicative of a specific characteristic, trait, disease, disorder or condition. Disclosed also is a kit comprising the assay.

### VI. BRIEF DESCRIPTION OF THE DRA WINGS

The accompanying drawings, which are incorporated in and constitute a part of this specification, illustrate several embodiments and together with the description illustrate the disclosed compositions and methods.

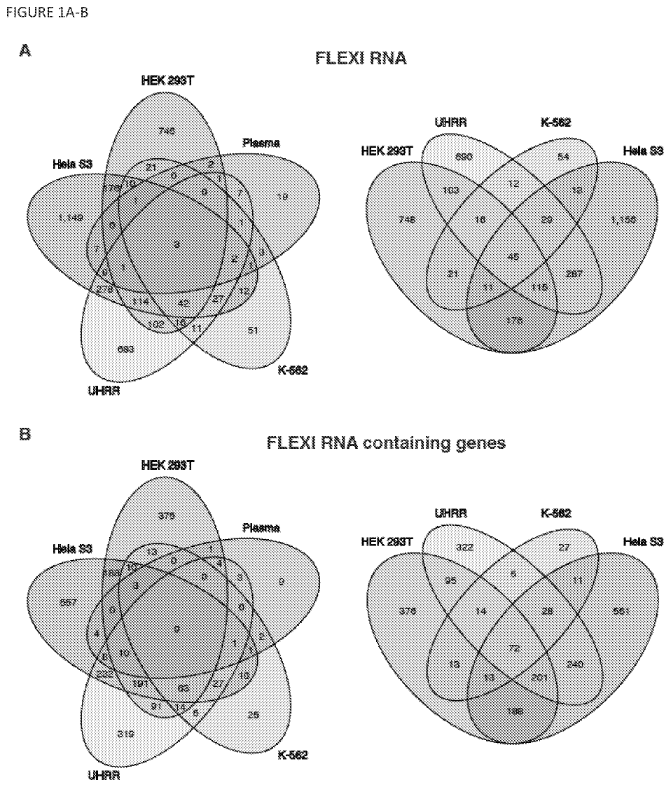

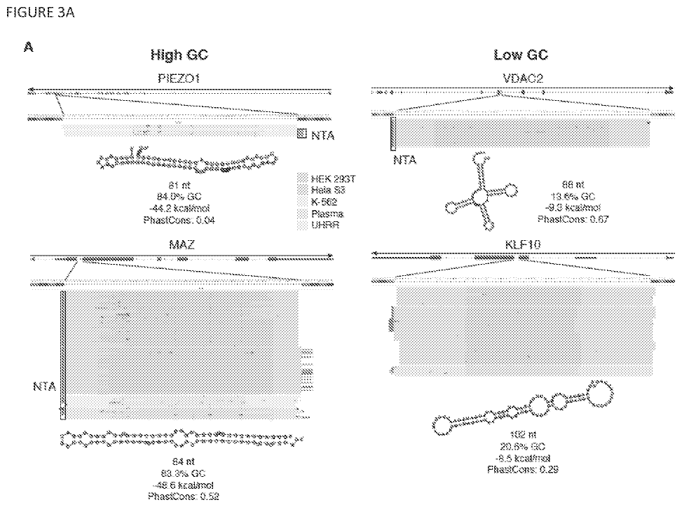

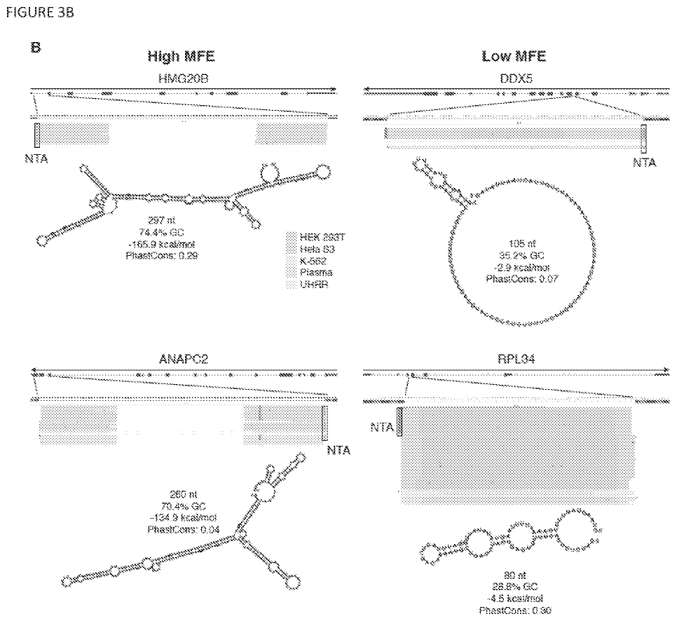

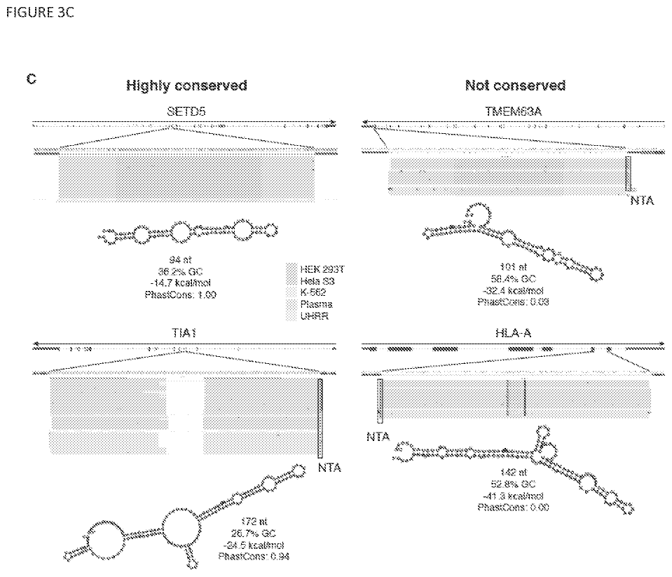

FIG. 1A-B are Venn diagrams showing the relationships between the full-length excised intron RNAs (FLEXI RNAs; intron RNAs 300 nt with 5′ and 3′ ends within 3 nts of annotated splice sites) and the genes encoding them identified by TGIRT-seq in human cellular and plasma RNA preparations. (A) FLEXI RNAs (5 reads) identified by TGIRT-seq in Universal Human Reference RNA (UHRR) and RNAs from HeLa S3 cells, HEK 293T cells, K-562 cells, and human plasma. (B) Genes in which the introns in panel (A) are encoded. A total 3,495 FLEXI RNAs were identified in five sources of RNA: UHRR (purchased from Agilent); HeLa S3 cell RNA (purchased from ThermoFisher); RNA extracted from cultured K-562 and HEK 293T cells, as described in Materials and Methods; and RNA extracted from commercial human plasma (Innovative Research, IPLA-N), as described in Materials and Methods. Analysis of combined TGIRT-seq datasets for each sample type (Table 1) identified 201 to 1,832 FLEXI RNAs in the different cellular RNA samples, with the lower number in K-562 cells reflecting lower read depth in the K-562 cell datasets. Most FLEXI RNAs (76%; 2,648 FLEXI RNAs) were specific to individual cell types or plasma. In these initial experiments, twenty four percent (847 FLEXI RNAs) were found in two or more sample types, but only three FLEXI RNAs were found in all five sample types (ACTB intron 5; SEMA4C intron 10, and JUP intron 11), and only 45 FLEXI RNAs were found in all sample types excluding plasma. The abundance of FLEXI RNAs (total FLEXI RNA counts per million; CPMs) were UHHR, 47.8; HeLa S3 cells, 88.5; K-562 cells, 53.4; HEK 293T cells, 180.4; and plasma, 19.4 CPM, with the most abundant FLEXI RNA in each sample type being present at 1.7-5.9 CPM. The majority of the genes from which FLEXI RNAs were detected (59%) also differed in different cell types. The identification of FLEXI RNAs by TGIRT-seq as full-length introns RNAs that have discrete 5′ and 3′ ends and extend from the 5′ to the 3′ splice site without an impediment that might be expected for a branch point (see FIG. 3) indicates that they are predominantly linear RNA molecules. Small subsets of the FLEXI RNAs (1.4 to 4% of FLEXI RNAs in cellular RNA preparations) corresponded to annotated mirtrons (pre-miRNAs/introns that are processed by Dicer into functional miRNA) (Berezikov et al., 2007; Ruby et al., 2007; Wen et al., 2015), and/or agotrons (intron RNAs that bind Ago2 and function as miRNAs (Hansen, 2018; Hansen et al., 2016)(Table 2). Full-length excised intron RNAs that correspond to mirtron pre-miRNAs or agotrons have not been detected previously by RNA-seq, presumably because their stable secondary structure makes them intractable to previously used RNA-seq methods employing retroviral reverse transcriptases.

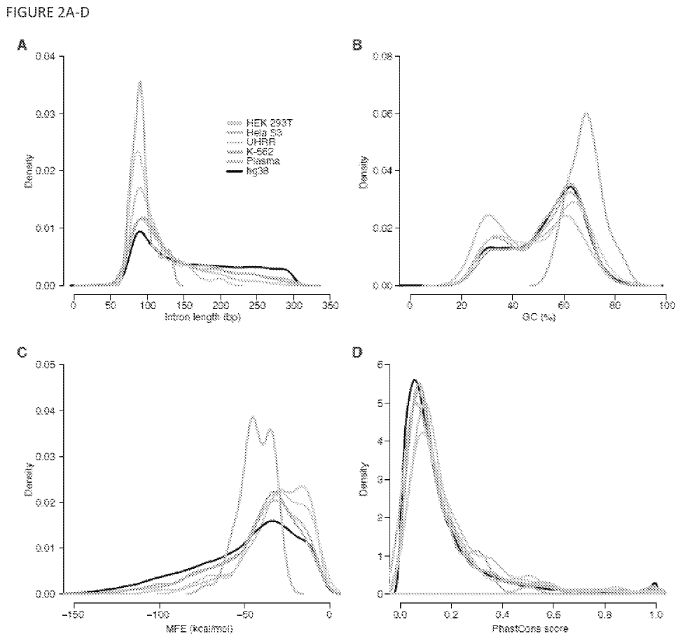

FIG. 2A-D shows density plots for several characteristics of the FLEXI RNAs that were detected in different human cell types and plasma, as well as for all annotated introns 300 nt in the hg38 human genome reference sequence. The latter totaled 51,664 different human introns that could potentially give rise to FLEXI RNAs. (A) Length. Most FLEXI RNAS are short (<150 nt), but those in whole-cell RNAs have a wider size distribution than those found in plasma. (B) GC content. FLEXI RNAs in cells have two peaks at-30 and 60% GC, whereas FLEXI RNAs in plasma have a single peak at −70% GC. (C) Minimum free energy (MFE; 11G) for the most stable secondary structure predicted by RNAfold (Zuker and Stiegler, 1981). Most FLEXI RNAs detected in plasma have a lower MFE (i.e., a more stable predicted secondary structure) than those detected in cells. (D) Evolutionary conservation. Most but not all FLEXI RNAs have low PhastCons scores indicating a low degree of evolutionary conservation. PhastCons scores were calculated for 27 primates including humans plus mouse, dog, and armadillo and downloaded from the University of California, Santa Cruz (UCSC) genome browser.

FIG. 3A-E shows IGV screenshots of read alignments for different types of FLEXI RNAs. Gene names are indicated at the top with an arrow indicating the 5′ to 3′ orientation of the encoded RNA Gene annotations are shown in the top track (exons, thick bars; introns, thin lines). The second track is expanded to show the relevant part of the gene map. Read alignments for FLEXI RNAs are shown below the expanded gene map and are color coded by cell type or plasma as indicated in the Figure. The most stable predicted secondary structure computed by RNAfold (Zuker and Stiegler, 1981) is shown below the read alignments along with length, GC content, calculated minimum free energy (11G) for the most stable predicted structure, and PhastCons score for 27 primates and three other species. (A) Examples of FLEXI RNAs having high or low GC content. (B) Examples of FLEXI RNAs having low or high predicted MFE. (C) Examples of FLEXI RNAs having high or low PhastCons scores. (D) Examples of long and short FLEXI RNAs. (E) Examples of FLEXI RNAs having non-canonical (non-GU-AG) 5′ and 3′ends. Most (>98.7%) of the detected FLEXI RNAs have canonical 5′-GU-AG-3′ ends, with 1.1% having 5′-GC-AG-3′ ends and small proportions having other 5′—and 3′ termini (FIG. 3E), similar to the proportions for all mammalian mRNA introns (Burset et al., 2000). Thus far, none of the detected FLEXI RNAs had S′-AU-AC-3′ ends characteristic of alternative spliceosome introns, which constitute −0.02% of human introns, but it remains possible that such FLEXI RNAs could be detected in larger datasets or in other cell types. Abbreviation: NTA, non-templated nucleotide that are added to the 3′ ends of cDNAs by TGIRT enzyme during TGIRT-seq, appearing as extra nucleotides at the 5′ end of the RNA sequence.

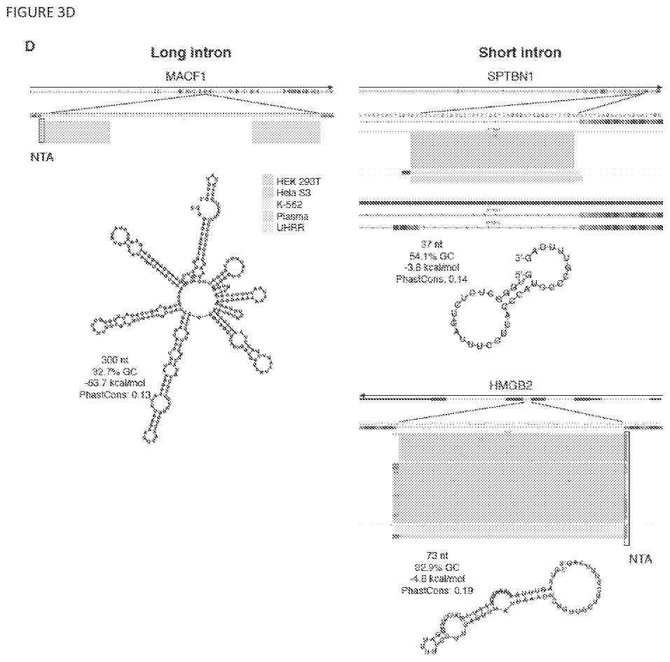

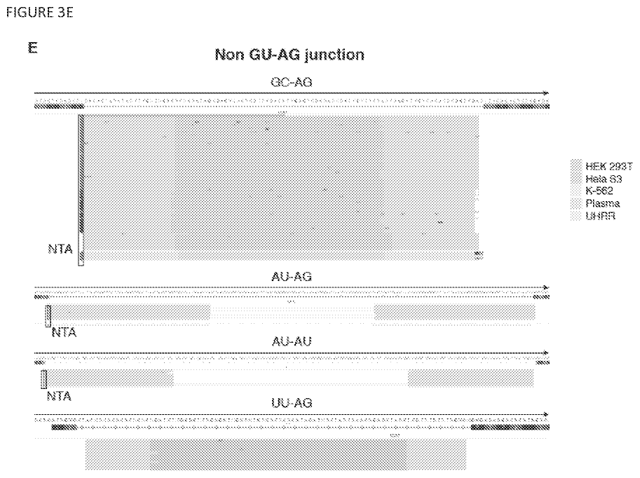

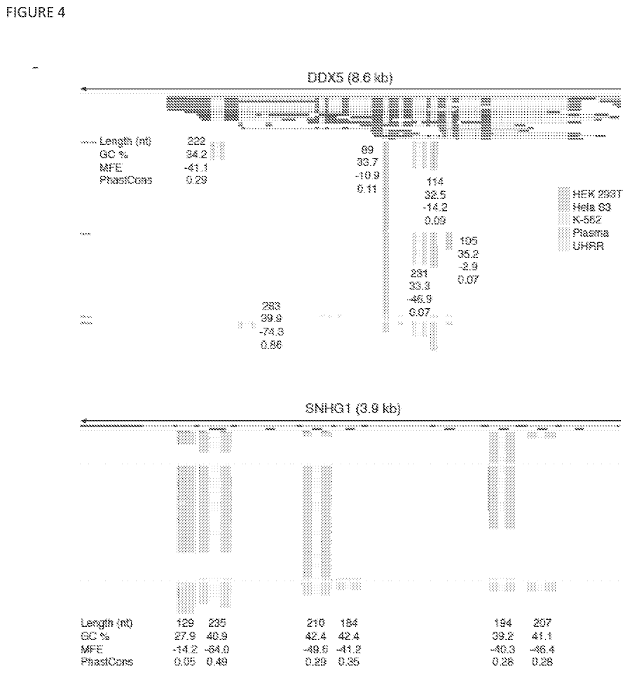

FIG. 4 shows examples of genes encoding multiple FLEXI RNAs. Gene name and length are indicated at the top with the arrow indicating the 5′ to 3′ orientation of the encoded RNA and gene annotations shown below (exons, thick bars; introns, thin lines). Read alignments for FLEXI RNAs are shown below the gene map and are color coded by cell type or plasma. Length, GC content, calculated MFE (11G) for the most stable secondary structure predicted by RNAfold (Zuker and Stiegler, 1981), and PhastCons score for 27 primates and three other species are indicated for each FLEXI RNA The relative abundance of different FLEXI RNAs encoded in a gene differs in different cell and tissue types, indicating that not only gene expression (transcription), but also alternative splicing or differential stability can contribute to the abundance and detection of FLEXI RNAs in different cells.

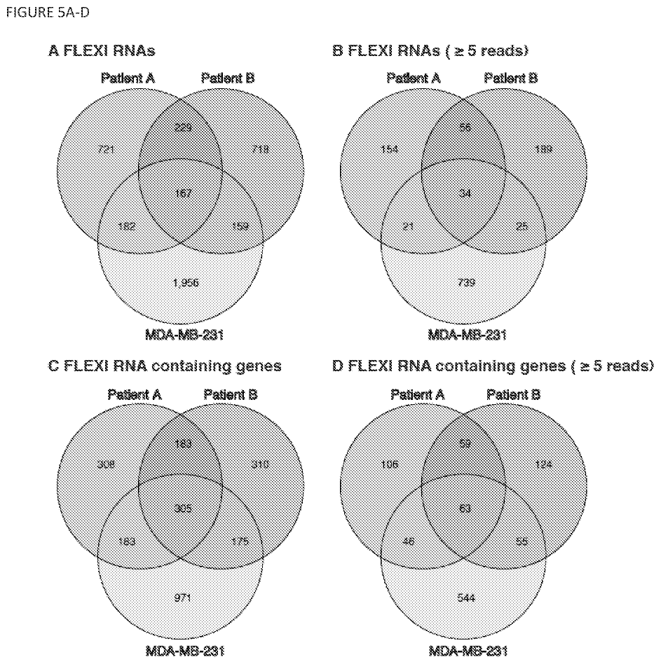

FIG. 5A-D shows Venn diagrams showing the relationships between detected FLEXI RNAs and the genes encoding them in matched cancer/normal breast tissue from two breast cancer patients. The patient RNAs were purchased from Origene. Patient A: PR+, ER+, HER2−, CR543839/CR562524; Patient B: PR unknown, ER−, HER2−, CR532030/CR560540).

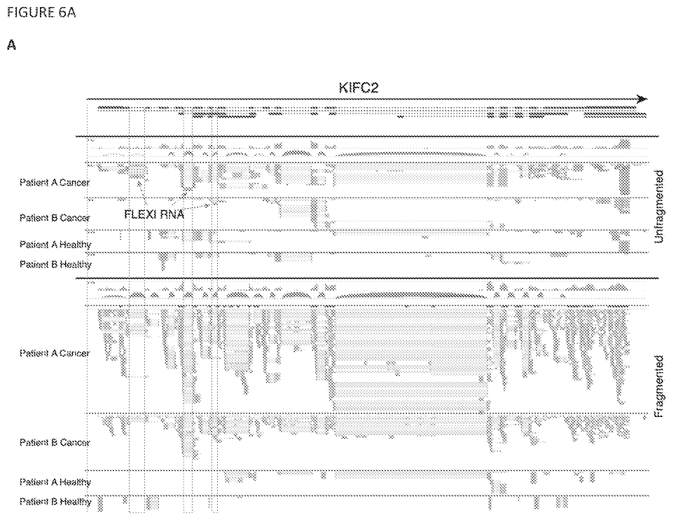

FIG. 6A-B shows IGV screenshots showing examples of FLEXI RNAs unique to cancer tissues from patient A or B. The gene name is indicated at the top with the arrow indicating the 5′ to 3′ orientation of the major transcript, and the gene map shown below (exons thick bars, introns, thin lines. Read coverage shown below the gene map was computed from combined datasets for different samples types (Table 1). Splice junction are shown below the coverage track with arcs connecting splice junctions from a single read. The thickness of the arc is proportional to the number of reads for that splice junction. Read alignments are shown below the splice junction track. FLEXI RNAs detected only in unfragmented RNA preparations from cancer tissue of patient A (top panel) or patient B (bottom panel) are highlighted in green boxes. The pattern of RNA fragments mapping within introns also varies between the healthy and cancer tissues in some cases. Chemically fragmented RNAs from the same healthy and cancer tissues were sequenced for comparison and IGV screen shots for those samples are shown below those for the non-chemically fragmented (i.e., unfragmented) RNA samples.

FIG. 7A-D shows characteristics of FLEXI RNAs in human cells and plasma. (A) UpSet plots of FLEXI RNAs and their host genes detected at 1 read in unfragmented RNA preparations from the indicated samples. (B) Scatter plots comparing log 2-transformed RPM of FLEXI RNAs and all transcripts of FLEXI host genes in different cellular RNA samples.rand rs. are Pearson and Spearman correlation coefficients, respectively. (C) Density plots of different characteristics of FLEXI RNAs in combined datasets for the UHRR, K-562, HEK-293T and HeLa S3 cellular RNA samples. Left panel, density plot showing the distribution of RNA fragment lengths mapping to FLEXIs in the cellular RNA samples (red line) compared to those mapping to other Ensemble GRCh38-annotated short introns; 300 nt in the same samples (dashed black line). Percent intron length was calculated from the read spans of TGIRT-seq reads normalized to the length of the corresponding intron. Introns encoding embedded snoRNAs or scaRNAs were removed for these comparisons to avoid interference from mature snoRNA reads. Middle panels, density distribution plots of length, GC content, and minimum free energy calculated for the most stable RNA secondary structure predicted by RNAfold for all FLEXIs detected in the cellular RNA (red) and plasma cell-free RNA (purple) samples, compared to other Ensemble GRCh38-annotated short introns; 300 nt detected in the same samples (dashed black line). Right panel, density distribution plots of phastCons scores of different categories of FLEXIs detected in the cellular RNA samples compared to all other Ensemble GRCh38 annotated short introns; 300 nt. PhastCons scores were the average PhastCons score across all intron bases calculated from multiple sequences alignment of 27 primates, including humans plus mouse, dog, and armadillo. (D) Density distribution plots of the abundance (RPM) of different categories of FLEXI RNAs color coded as indicated in the Figure in different cellular RNA samples. Only full-length FLEXI RNA reads with 5′ and 3′ ends within 3 nts of annotated splice sites were used in calculating abundances. The abundance distribution of annotated mature snoRNAs in the same samples is shown for comparison (dashed line), as are the positions (arrows) of different sncRNAs detected by TGIRT-seq in the same samples, including two low abundance, biologically relevant CID box snoRNAs (SNORD74: 0.01-0.1 RPM; SNORD78: 0.02-0.3 RPM) (Martens-Uzunova et al. 2015; Oliveira et al. 2021).

FIG. 8A-D shows IGV screenshots showing read alignments for FLEXI RNAs. Gene names are at the top with the arrow below indicating the 5′ to 3′ orientation of the encoded RNA followed by tracts showing gene annotations (exons, thick bars; introns, thin lines), sequence, and read alignments for FLEXI RNAs color coded by sample type as indicated in the Figure (bottom right). (A) Long and short FLEXI RNAs; (B) FLEXI RNAs having high and low GC content; (C) FLEXI RNAs having low and high minimum free energies (MFEs) for the most stable RNA secondary structure predicted by RNAfold; (D) FLEXI RNAs showing cell-type specific differences due to alternative splicing and differential stability of FLEXI RNAs encoded by the same gene. The most stable secondary structure predicted by RNAfold is shown below the read alignments (panels A-Conly) along with intron length, GC content, calculated MFE, and PhastCons score for 27 primates and three other species. In panel D, gene maps for the different RNA isoform generated by alternative splicing of FLEXI RNAs are shown at the bottom Mismatched nucleotides in boxes at the 5′ end of the RNA sequence are due to non-templated nucleotide addition (NTA) to the 3′ end cDNAs by TGIRT-III during TGIRT-seq library preparation. Some_M4Z FLEXIs (panel B) have a non-coded 3′ A or U tail.

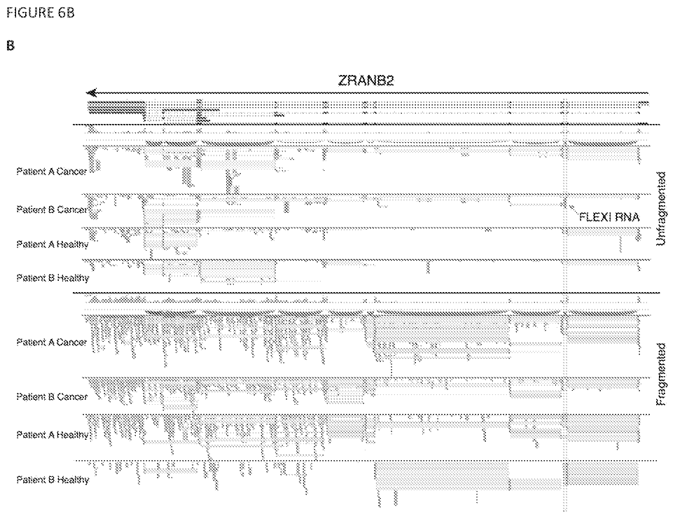

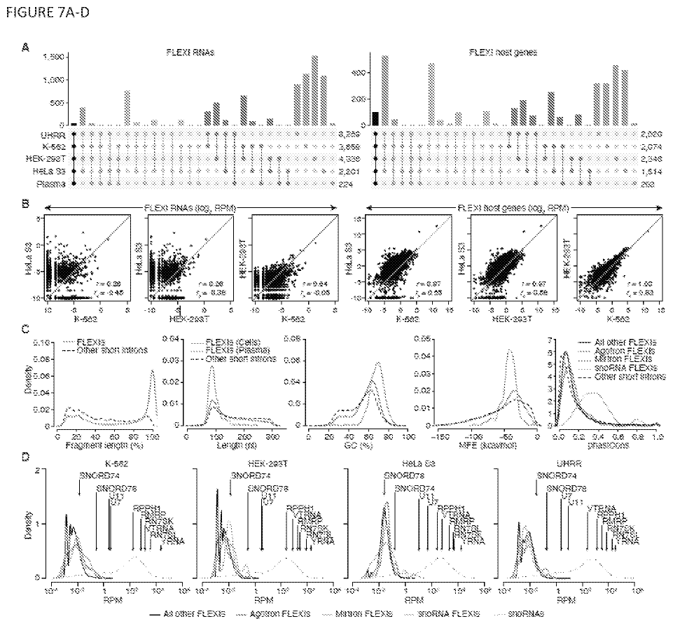

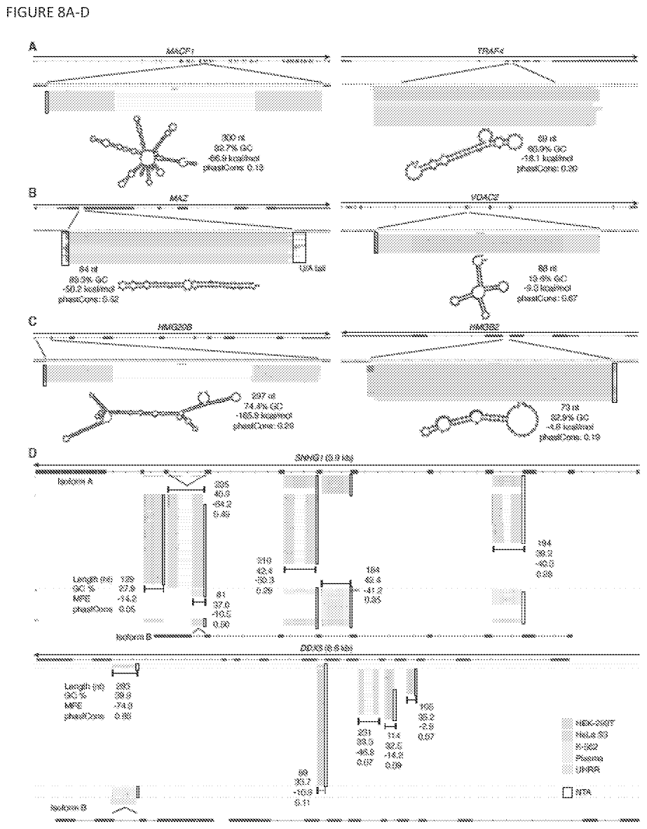

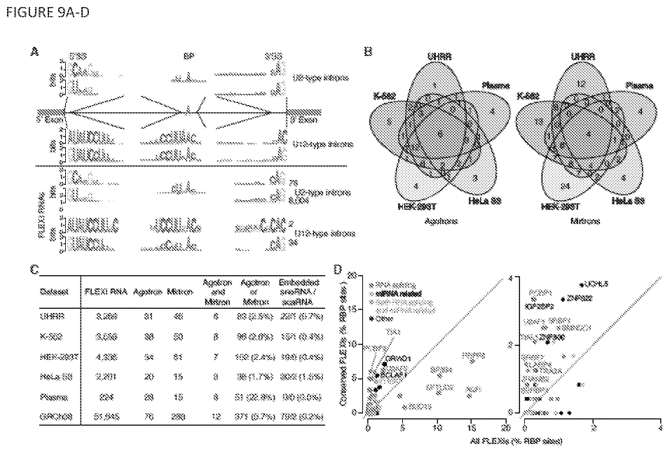

FIG. 9A-D shows FLEXI RNA splice-site and branch-point consensus sequences, FLEXI RNAs annotated as mirtrons or agotrons or encoding an embedded snoRNAs in different sample types, and RBP-binding sites enriched in highly conserved FLEXI RNAs. (A) 5′—and 3′-splice sites (5′SS and 3′SS, respectively) and branch-point (BP) consensus sequences of FLEXI RNAs compared to those of human major (U2-type) and minor (U12-type) spliceosomal introns. The number of FLEXIs matching each consensus sequence is indicated to the right. The remaining FLEXIs have non-canonical 5′—and 3′-splice site sequences. (B) Venn diagrams showing the relationships between FLEXI RNAs corresponding to annotated agotrons (left) or mirtrons (right) detected in different sample types. FLEXI RNAs annotated as both a mirtron and an agotron are included in both Venn diagrams. (C) Numbers and percentages of detected FLEXI and short introns 300 nt in the human genome (GRCh38) corresponding to annotated agotrons or mirtrons or encoding embedded snoRNAs in different sample types. “Agotron and Mirtron” indicates introns annotated as both an agotron or mirtron, and “Agotron or Mirtron” indicates the total number and percentage of introns annotated as either or both an agotron or mirtron. The number of embedded snoRNAs that are small Cajal body-specific snoRNAs (scaRNAs) is also indicated. (D) Scatter plots showing the relative abundance (percentage) of annotated binding sites for different RBPs in highly conserved FLEXI RNAs (phastCons score 0.99; n=44) compared to that in all detected FLEXIs RNAs in the cellular and plasma samples. RBP-binding site annotations are from the ENCODE 150 RBP eCLIP dataset with irreproducible discovery rate (IDR) and AGOI-4 and DICER PAR-CLIP datasets. The scatter plot on the right is an enlargement of the Oto 4% abundance region of the scatter plot on the left. RBPs whose relative abundance was significantly different between the highly conserved FLEXIs and all FLEXIs (p:s; 0.05 calculated by Fisher's exact test) are labeled with the name of the RBP color coded by protein function: red, RNA splicing related; orange, miRNA related; blue, both RNA splicing and miRNA related; black, Other, RBPs whose primary function is not RNA splicing or miRNA related.

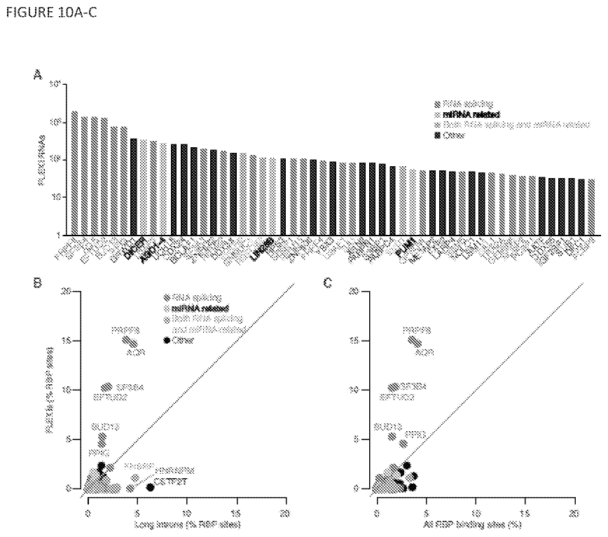

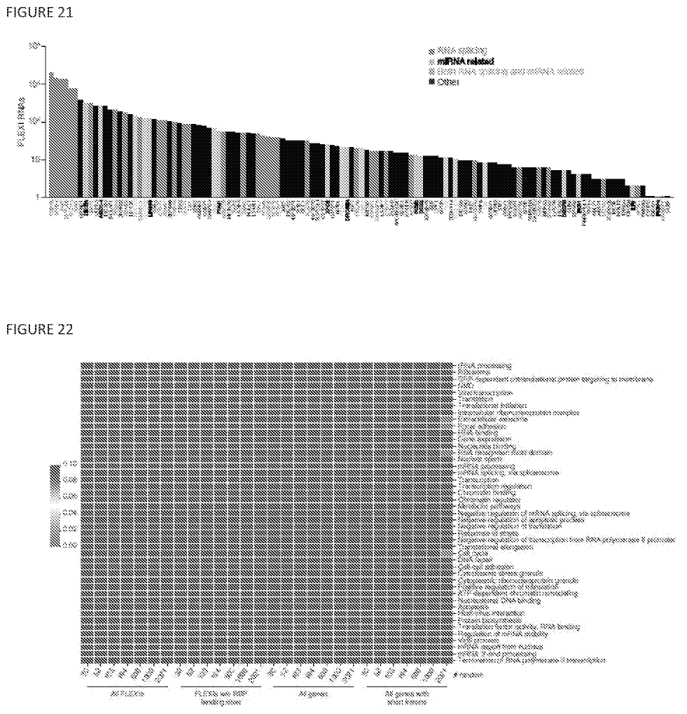

FIG. 10A-C shows Protein-binding sites in FLEXI RNAs. (A) Bar graph showing the number of detected FLEXIs in the cellular and plasma RNA datasets that have an experimentally identified RBP binding site for the indicated RBP. Only RBPs that bind 30 or more different FLEXIs are shown; a bar graph for the complete set of detected FLEXIs is shown in FIG. 21. (Band C) Scatter plots comparing the relative abundance of RBP-binding sites in the detected FLEXI RNAs with those in all annotated longer introns>300 nt in GRCh38 (panel B) or all RBP-binding sites in the ENCODE 150 RBP eCLIP dataset with IDR plus the AGOI-4 and DICER PAR-CLIP dataset using GRCh38 as the reference sequence (panel C). RBPs whose relative abundance was 4% and significantly different between the compared groups (p :s; 0.05 calculated by Fisher's exact test) are indicated by the name of the RBP color coded by protein function as indicated in the keys in panels A and B.

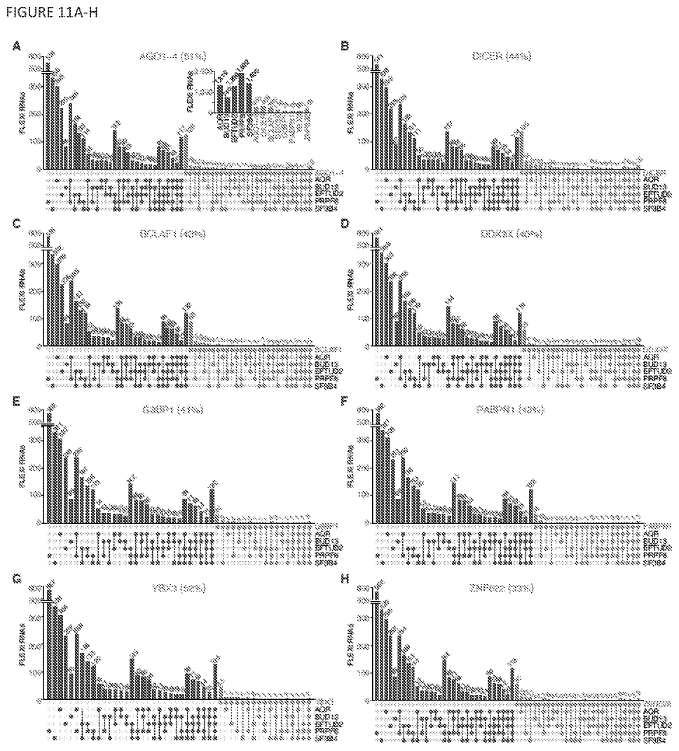

FIG. 11A-H shows UpSet plots identifying RBPs that bind FLEXI RNAs lacking annotated binding sites for core spliceosomal proteins. (A) and (B) AGOI-4 and DICER, respectively. (C-1) RBPs that have no known RNA splicing- or miRNA-related function. Each plot compares the FLEXI RNAs in the cellular and plasma RNA datasets that contained an annotated binding site for the RBP of interest in the CLIP-seq datasets to those that contained annotated binding sites for any of five ubiquitous core spliceosomal proteins (AQR, BUD13, EFTUD2, PRPF8, and SF3B4) in those datasets (black). In each case, a substantial proportion (29-55%) of the FLEXI RNAs bound by the RBP of interest lacked an annotated binding site for any of the five core spliceosomal proteins. The inset in the top left UpSet plot shows the total number of different FLEXIs that contained an annotated binding site for each of the RBPs. Similar distinct classes of FLEXIs that bind the indicated RBP but lack annotated binding sites for the spliceosomal proteins were found for DDX55 (55%), IGF2BP1 (52%), FRX2 (47%), ZNF800 (33%), LARP4 (33%), RPS3 (33%), UCHL5 (32%), METAP2 (31%), LSM1I (30%), and GRWD1 (29%).

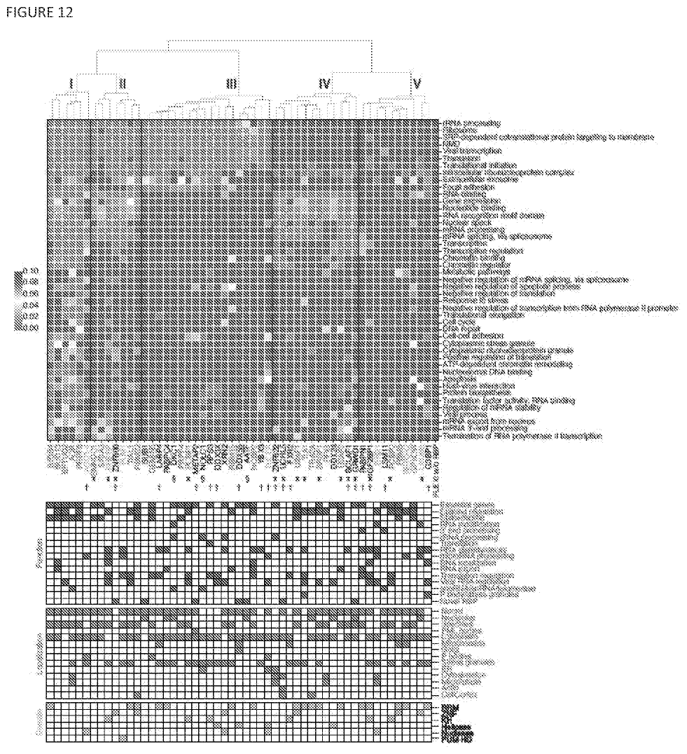

FIG. 12 shows heatmap of GO terms enriched in host genes of FLEXI RNAs containing binding sites for different RBPs. GO enrichment analysis was performed using DAVID bioinformatics tools. and clustering was performed based on the adjusted p-value for each enriched category using Seaborn ClusterMap. The function, cellular localization, and protein motif information for the RBPs are summarized below using information from (Van Nostrand et al. 2020) supplemented by information from mammalian RNA granule and stress granule protein databases (Nunes et al. 2019; Youn et al. 2019), and AGOI-4 and DICER information from the UniProt database (The UniProt Consortium 2018). RBP are color coded by protein function: red, RNA splicing-related function; orange, miRNA-related functions blue, both an RNA splicing- and a miRNA-related function; black, RBPs whose primary function is not RNA splicing- or miRNA-related. *: RBPs that bind FLEXI RNAs with phastCons 0.99; §: three RBPs that bind FLEXIs with relatively low GC content including 41 of 43 FLEXIs that encode embedded snoRNAs; t: RBPs that bind a substantial proportion of the FLEXI RNAs (29-55%) that lacked annotated binding sites for any of the five most ubiquitous core spliceosomal proteins (AQR, BUD13, EFTUD2, PRPF8, and SF384).

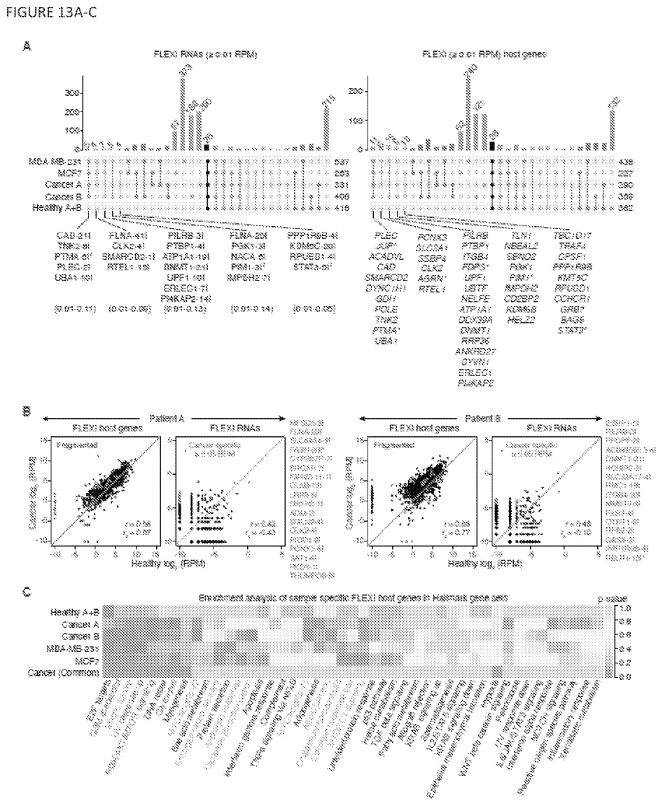

FIG. 13A-C shows FLEXI RNAs in breast cancer tumors and cell lines. (A) UpSet plots of FLEXI RNAs and FLEXI host genes detected at 0.01 RPM in unfragmented RNA preparations from matched cancer/healthy breast tissues from patients A (PR+, ER+, HER2−) and B(PR unknown, ER−, HER2−) and breast cancer cell lines MDA-MB-231 and MCF7. Different FLEXI RNAs from the same host gene were aggregated into one entry for that gene. FLEXI RNAs and FLEXI host genes are listed below some sample groups in descending order of RPM, with the RPM indicated in parentheses at the bottom. (B) Scatter plots comparing log 2 transformed RPM of FLEXI RNAs in unfragmented RNA preparations and all transcripts from FLEXI host genes in chemically fragmented RNA preparations from cancer and healthy breast tissues from patients A and B. FLEXI RNAs present at 0.05 RPM and detected in at least two replicate libraries from the cancer tissue but not in the matched healthy tissue indicated in red and listed to the right of the scatter plots. (C) GO enrichment analysis of genes encoding detected FLEXI RNAs in 50 hallmark gene sets (MSigDB) in cancer samples and combined patient A+B healthy tissue samples. Names of pathways significantly enriched (p 0.05) in all or at least one cancer sample are in red and orange, respectively. In panels A and B, oncogenes and FLEXI RNAs originating from oncogenes are denoted with an asterisk.

FIG. 14A-E shows oncogene FLEXI RNAs in breast cancer tumors and cell lines. (A and B) UpSet plots of upregulated (A) and downregulated (B) oncogene FLEXI RNAs in unfragmented RNA preparations from matched cancer/healthy breast tissues from patients A and Band breast cancer cell lines MDA-MB-231 and MCF7. FLEXIs originating from the FASN gene are highlighted in red. (C and D) UpSet plots of upregulated (C) and downregulated (D) tumor suppressor gene (TSG) FLEXI RNAs in the same unfragmented RNA preparations. Up and down regulated FLEXI RNAs were defined as those with an RPM-fold change 2. The most abundant oncogene or TSG FLEXI RNAs (up to a limit of 10) are listed below some sample groups, with the range of RPM values indicated in parentheses at the bottom. (E-G) Scatter plot comparing the relative abundance (percentage) of different RBP-binding sites in oncogene FLEXIs that are upregulated only in MCF7 cells, only in MDA-MB-231 cells, or in all four cancer samples compared to the abundance of all detected FLEXIs in the same sample or samples. For each pair of plots, the RBPs whose relative abundance is significantly different (p :s; 0.05 calculated by Fisher's exact test) are shown in red with names labeled.

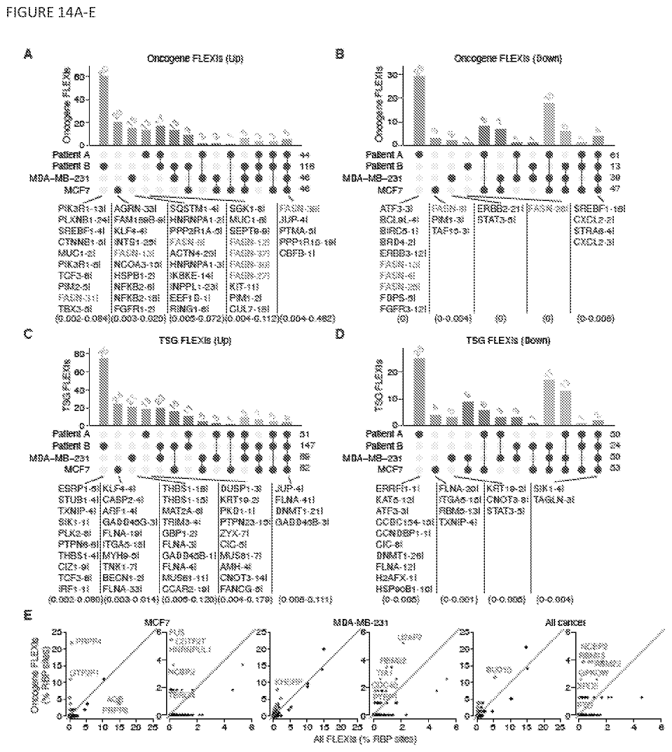

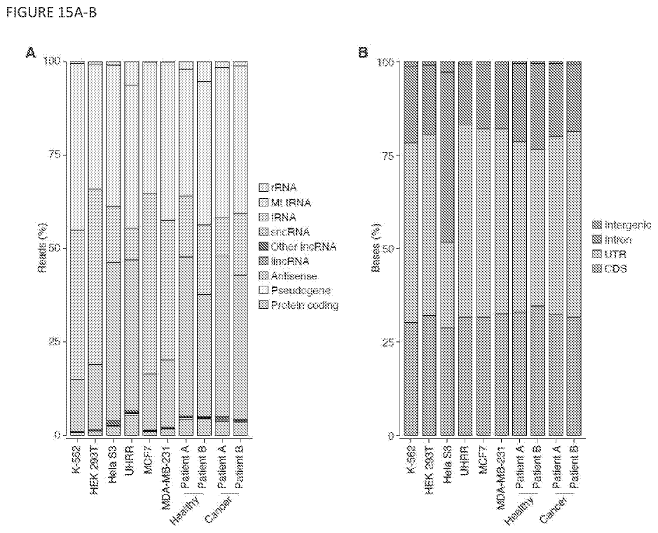

FIG. 15A-B shows TGIRT-seq of ribodepleted unfragmented cellular RNA (A) Stacked bar graphs showing the percentage of reads in the combined datasets for the indicated samples in this study that mapped to different categories of annotated genomic features in the GRCh38 human genome reference sequence. Genomic features follow Ensembl GRCh38 Release 93 annotations. rRNA includes cellular and mitochondrial (Mt) rRNAs; protein coding includes protein-coding transcripts from both the nuclear and Mt genomes. (B) Stacked bar graphs showing the percentage of bases that mapped to different regions of the sense strand of protein-coding genes. CDS, coding sequences; intergenic, regions upstream or downstream of transcription start and stop sites annotated in RefSeq; intron, intronic regions; and UTR, 5′—or 3′-untranslated regions; C, tumor tissue from breast cancer patients A or B; H, neighboring healthy tissue from the same patient.

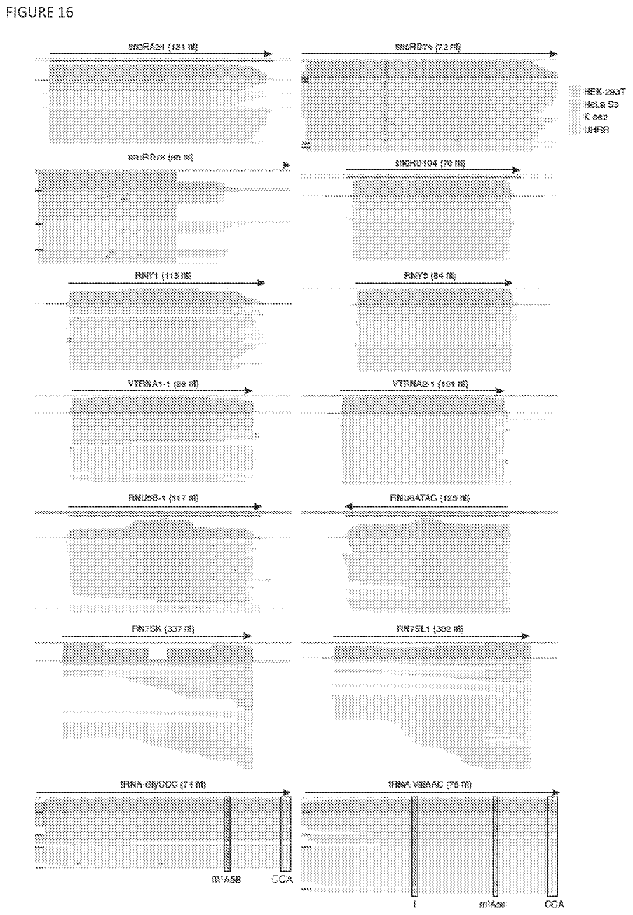

FIG. 16 shows integrative Genomics Viewer (IGV) screenshots showing examples of sncRNAs detected in ribodepleted intact (non-chemically fragmented) cellular RNAs by TGIRT-seq. After mapping individual datasets to a customized set of sncRNA references sequences that included mature tRNAs with post-transcriptionally added 3′ CCAs and to the Ensembl GRCh38 Release 93 human genome reference sequence, as described in Methods, individual datasets were combined and alignments for the indicated sncRNAs were displayed in IGV. Coverage at each position along the gene is shown in the top tract, and read alignments are shown below with reads down sampled to a maximum of 100 reads for display when necessary. Gray represents bases that match the reference base. Other colors indicate bases that do not match the reference base (thymidine, adenosine, cytidine, and guanosine). Misincorporation at known sites of tRNA post-transcriptionally modified bases are highlighted in the alignments: m¹ A58: 1-methyladenosine at position 58; I: inosine.

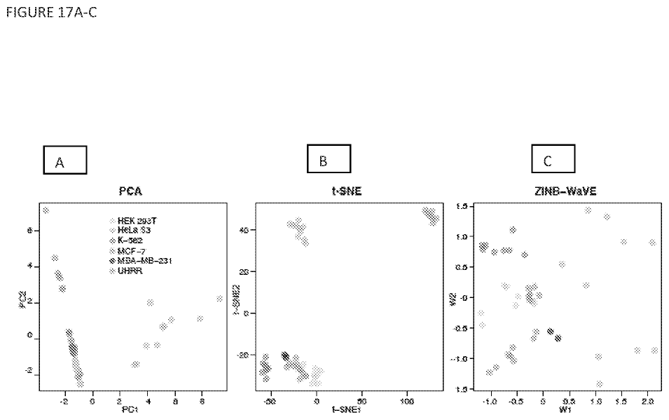

FIG. 17A-C shows PCA, PCA-initialized t-SNE, and ZINB-WaVE analysis of FLEXI RNAs detected in different replicates of all ribodepleted intact cellular RNA datasets in this study (Table 4). The plots show sample clustering based on all FLEXI RNAs detected at read in these datasets. Different cell types are color-coded as indicated in the Figure, with each dot of the same color representing a replicate for that cell type.

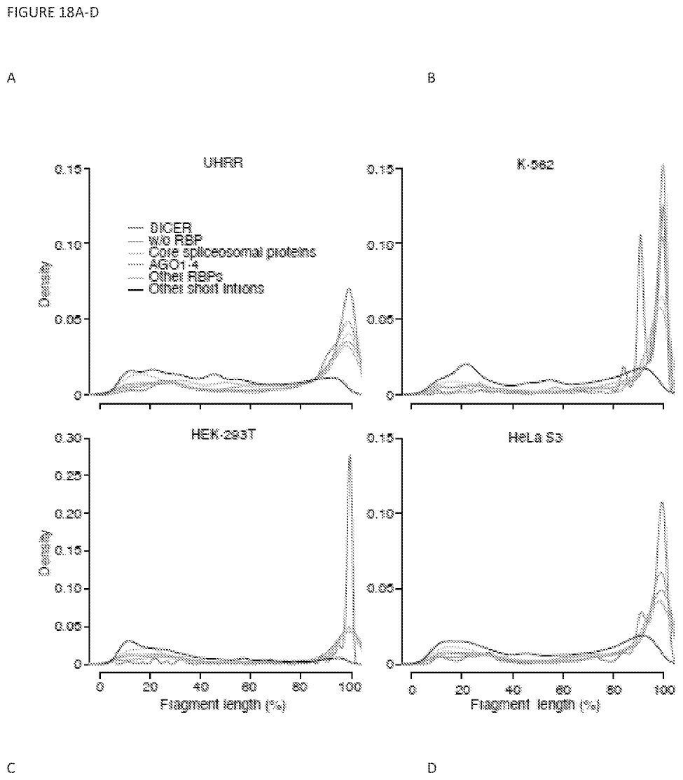

FIG. 18A-D shows density plots showing the distribution of RNA fragment lengths for different subcategories of FLEXIs in each of the cellular RNA samples. % intron length was calculated from the read span of TGIRT-seq reads normalized for the length of each intron. FLEXIs and short introns with embedded snoRNA or scaRNA were removed prior to calculating the distributions to avoid interference from mature snoRNA reads. Separate plots are shown for FLEXI RNAs containing annotated binding sites for AGO1-4, DICER, five core spliceosome proteins (AQR, BUD13, EFTUD2, PRPF8 and SF3B4), other annotated RBPs, FLEXIs without annotated RBP-binding sites in the searched datasets, and all other GRCh38-annotated short introns (300 nt). The different intron types are color coded as shown in the key in the top left plot.

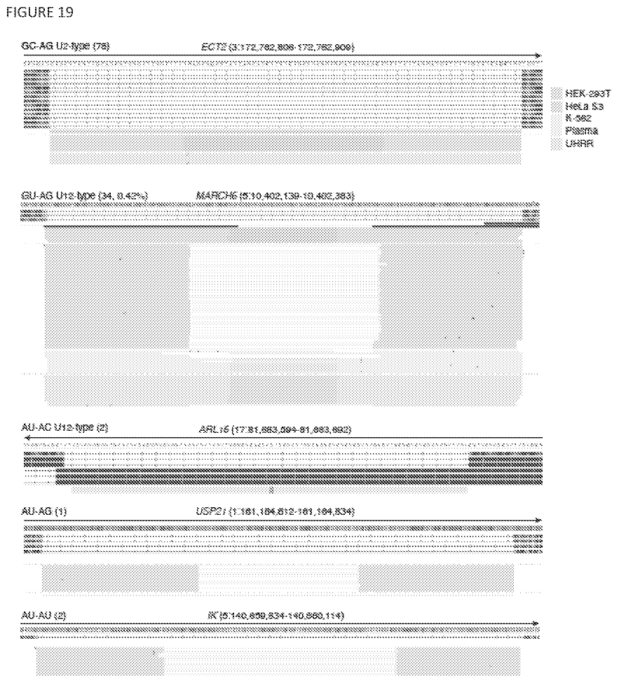

FIG. 19 shows IGV screenshots showing read alignments for FLEXI RNAs having non-GU-AG 5′—and 3′-splice sites. Dinucleotides at the 5′—and 3′-ends of the intron are indicated at the upper left with the number of introns in that category indicated in parentheses. Gene name and genomic coordinates of the FLEXI are shown at the top with the arrow below indicating the 5′ to 3′ orientation of the encoded RNA followed by tracts show the genomic sequence and gene annotations for different transcript isoforms (exons, thick bars; introns, thin lines). Read alignments for FLEXI RNAs are shown below the tracts and are color coded by sample type as indicated in the key at the upper right.

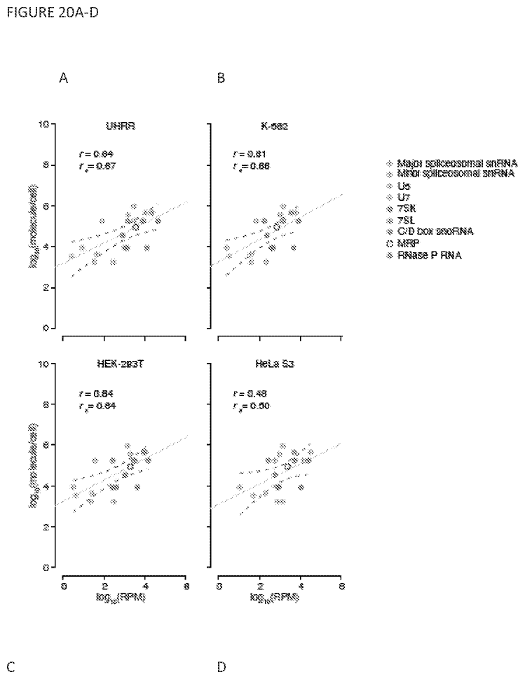

FIG. 20A-D shows plots showing the relationship between the abundance of sncRNAs detected by TGIRT-seq and copy number per cell values for human sncRNAs reported in the literature. The abundance of sncRNAs detected by TGIRT-seq (RPM) and literature values for their copy number per cell (Tycowski et al. 2006) were log 10 transformed and plotted. Pearson (r) and Spearman (rs) correlation coefficients are shown in the upper left. Linear regression was modeled for each cell type and plotted as light blue line, with the 95% confidence interval of the linear regression plotted as blue dashed lines. Major spliceosomal snRNAs used in the linear regression were U1, U2, U4, U5, and U6; minor spliceosomal snRNAs were U1 1, U12, U4ATAC, and U6ATAC; and CID box snoRNAs were SNORD3, SNORD13, SNORD14, SNORD22, and SNORD118 (Table 5) (Tycowski et al. 2006).

FIG. 21 shows bar graph showing the number of detected FLEXIs that have an experimentally identified RBP-binding site for the indicated RBP. FLEXIs are color coded by type as indicated in the key at the upper right.

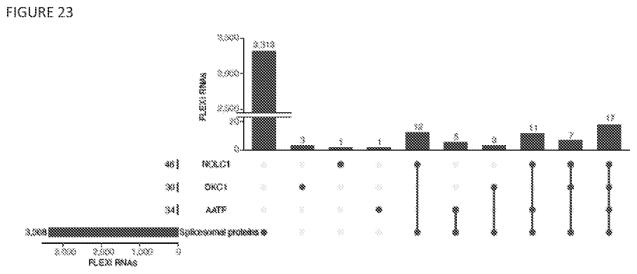

FIG. 22 shows GO term enrichment of randomly sampled FLEXI host and other genes. Random samples were taken from lists of all FLEXIs, FLEXIs without RBP-binding sites, all annotated genes, or all genes containing short introns (300 nt) in GRCh38. For each category, the number of included introns in each randomly selected list corresponded to the minimum, maximum, and quartile numbers of FLEXIs bound by different RBPs in FIG. 12 plus lists of 500 and 1,000 introns to better sample the full range of list sizes. Enrichment for the same GO terms as in FIG. 12 is shown as a heatmap of average p-values for three replicates of each randomly sampled list, with red corresponding to significant p-values 0.05 and blue indicating non-significance.

FIG. 23 shows UpSet plots of FLEXI RNAs bound by AATF, DKC1, NOLC1. Each plot compares the FLEXI RNAs in the cellular RNA datasets that contained an annotated binding site for one of the above RBPs to those that contained an annotated binding site for any of five most ubiquitous core spliceosomal proteins (AQR, BUD13, EFTUD2, PRPF8, and SF384) grouped as one entry.

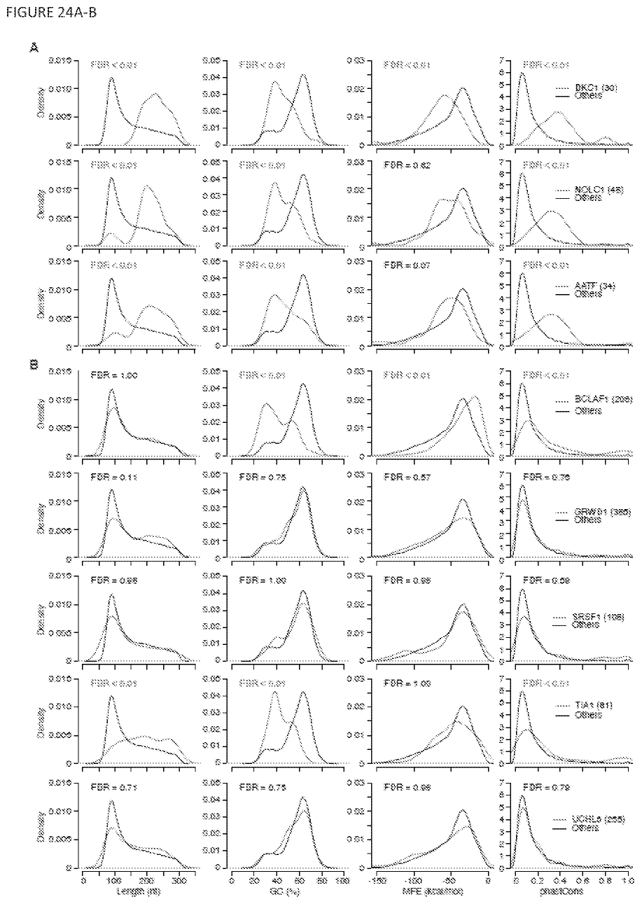

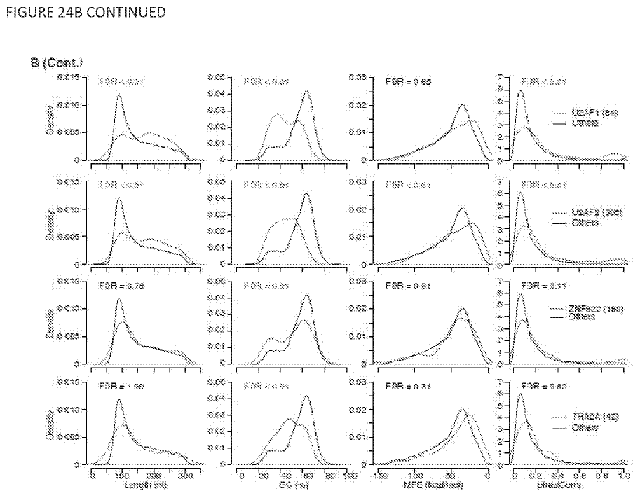

FIG. 24A-B shows density distribution plots comparing different characteristics of FLEXI RNAs containing a binding site for the indicated RBP (red) to those for all other detected FLEXIs (black). (A) Three RBPs in cluster III of FIG. 12 that bind FLEXI RNAs with relatively low GC content and above average phastCons scores. (B) RBPs in cluster IV whose binding sites are enriched in highly conserved FLEXIs and/or bind FLEXI RNAs with relatively low GC content. For each plot, the same number of FLEXI RNAs as those bound by the specific RBP of interest were randomly selected from other detected FLEXIs and used the calculate a density distribution for the same characteristic, and a p-value comparing the two density distributions was calculated by Wilcoxon test. This process was repeated 100 times, and a false discovery rate (FDR) was calculated as the probability of p-value:s; 0.05. FDRs:s; 0.01 are highlighted.

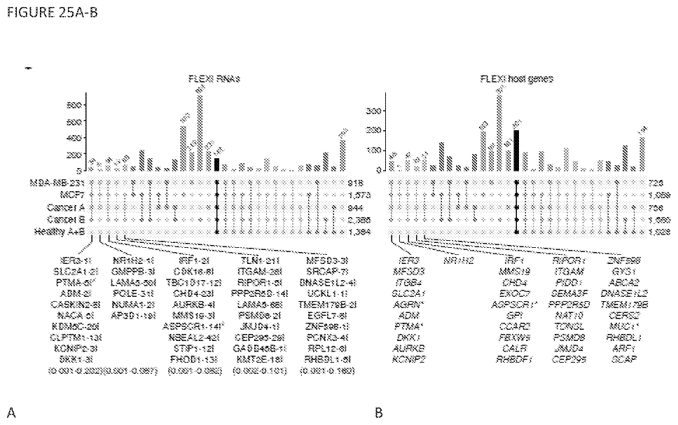

FIG. 25A-B shows UpSet plots of FLEXI RNAs detected at 2 1 read and their host genes in unfragmented RNA preparations from tumor and healthy breast tissues from patients A and Band breast cancer cell lines. Different FLEXI RNAs detected at 2 1 read from the same host gene were aggregated into one entry for that gene. FLEXI RNAs and FLEXI host genes are listed below some sample groups in descending order of RPM, with the RPM range of the detected FLEXIs indicated in parentheses at the bottom. Oncogenes and FLEXIs originating from oncogenes are indicated with an asterisk.

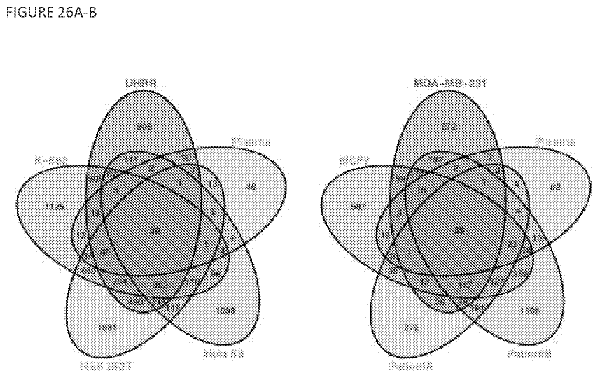

FIG. 26A-B are Venn diagrams showing the relationships between FLEXI RNAs (excised linear intron RNAs 300 nt with 5′ and 3′ ends within 3 nt of annotated splice sites) detected by TGIRT-seq (1 read) in RNAs from different human cell lines, universal human reference RNA (UHHR) and plasma (left panel) and between breast cancer cell lines MDA-MB-231 and MCF7, breast cancer tumor tissues from patients A and B, and plasma (right panel).

### VII. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may of course vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that these data, represents endpoints and starting points, and ranges for any combination of the data points. For example, if a particular data point “O” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

“Treat,” “treating,” “treatment,” and grammatical variations thereof as used herein, include the administration of a composition, or surgery, radiation, psychological treatments, or other types of treatments known to those of skill in the art, with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs), during early onset (e.g., upon initial signs and symptoms), or after an established development of disease or disorder. Prophylactic administration can occur for day(s) to years prior to the manifestation of symptoms of an infection.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of′ when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of’ shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.” A control can be used to compare the results of an assay to a standard, for example, a non-diseased state.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per seas well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

“Biological sample” as used herein may mean a sample of biological tissue or fluid that comprises FLEXI RNAs. Such samples include, but are not limited to, tissue or fluid isolated from subjects. Biological samples may also include sections of tissues, such as biopsy and autopsy samples, frozen or fixed sections taken for histologic purposes, blood, plasma, serum, sputum, stool, tears, mucus, hair, and skin. Biological samples also include explants and primary and/or transformed cell cultures derived from animal or patient tissues. Biological samples may also be blood, a blood fraction, urine, effusions, ascitic fluid, amniotic fluid, saliva, cerebrospinal fluid, cervical secretions, vaginal secretions, endometrial secretions, gastrointestinal secretions, bronchial secretions, sputum, cell line, tissue sample, or secretions from the breast. A biological sample may be provided by removing a sample of cells from a subject but can also be accomplished by using previously isolated cells (e.g., isolated by another person, at another time, and/or for another purpose). Archival tissues, such as those having treatment or outcome history, may also be used.

The term “cancer” is meant to include all types of cancerous growths or oncogenic processes, metastatic tissues or malignantly transformed cells, tissues, or organs, irrespective of histopathologic type or stage of invasiveness. Examples of cancers are given below.

The term “classification” refers to a procedure and/or algorithm in which individual items are placed into groups or classes based on quantitative information on one or more characteristics inherent in the items (referred to as traits, variables, characters, features, etc.) and based on a statistical model and/or a training set of previously labeled items. A “classification tree” is a decision tree that places categorical variables into classes.

As used herein, a “data processing routine” refers to a process that can be embodied in software that determines the biological significance of acquired data (i.e., the ultimate results of an assay or analysis). For example, the data processing routine can make determination of tissue of origin based upon the data collected. In the systems and methods herein, the data processing routine can also control the data collection routine based upon the results determined. The data processing routine and the data collection routines can be integrated and provide feedback to operate the data acquisition, and hence provide assay-based judging methods.

As used herein the term “data structure” refers to a combination of two or more data sets, applying one or more mathematical manipulations to one or more data sets to obtain one or more new data sets, or manipulating two or more data sets into a form that provides a visual illustration of the data in a new way. An example of a data structure prepared from manipulation of two or more data sets would be a hierarchical cluster.

“Detection” means detecting the presence of a component in a sample. Detection also means detecting the absence of a component. Detection also means measuring the level of a component, either quantitatively or qualitatively.

“Differential expression” means qualitative or quantitative differences in the temporal and/or cellular gene expression patterns within and among cells and tissue. Thus, a differentially expressed gene may qualitatively have its expression altered, including an activation or inactivation, in, e.g., normal versus disease tissue. Genes may be turned on or turned off in a particular state, relative to another state thus permitting comparison of two or more states. A qualitatively regulated gene may exhibit an expression pattern within a state or cell type which may be detectable by standard techniques. Some genes may be expressed in one state or cell type, but not in another. Alternatively, the difference in expression may be quantitative, e.g., in that expression is modulated, either up-regulated, resulting in an increased amount of transcript, or down-regulated, resulting in a decreased amount of transcript. The degree to which expression differs need only be large enough to quantify via standard characterization techniques, such as expression arrays, quantitative reverse transcriptase PCR, northern analysis, real-time PCR, in situ hybridization and RNase protection.

The term “expression profile” is used broadly to include a genomic expression profile, e.g., an expression profile of FLEXI RNAs. Profiles may be generated by any convenient means for determining a level of a nucleic acid sequence. The expression profile may include expression data for 5, 10, 20, 25, 50, 100 or more FLEXI-RNA sequences. According to some embodiments, the term “expression profile” means measuring the abundance of the nucleic acid sequences in the measured samples.

“Expression ratio” as used herein refers to relative expression levels of two or more nucleic acids as determined by detecting the relative expression levels of the corresponding nucleic acids in a biological sample.

“Fragment” is used herein to indicate a non-full length part of a nucleic acid. Thus, a fragment is itself also a nucleic acid.

“Gene” used herein may be a natural (e.g., genomic) or synthetic gene comprising transcriptional and/or translational regulatory sequences and/or a coding region and/or non-coding sequences (e.g., FLEXI RNAs). A gene may be an mRNA or cDNA corresponding to the coding regions (e.g., exons and miRNA) optionally comprising 5′—or 3′-untranslated sequences linked thereto, or to non-coding regions, such as FLEXI RNAs. A gene may also be an amplified nucleic acid molecule produced in vitro comprising all or a part of the coding region and/or 5′-or 3′-untranslated sequences linked thereto.

“Identical” or “identity” as used herein in the context of two or more nucleic acids or polypeptide sequences may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Nucleic acid” or “oligonucleotide” or “polynucleotide” used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

As used herein, the phrase “reference expression profile” refers to a criterion expression value to which measured values are compared in order to determine whether the measured values are indicative of a specific characteristic, trait, disease, disorder or condition. The reference expression profile may be based on the abundance of the nucleic acids, or may be based on a combined metric score thereof.

“Variant” used herein to refer to a nucleic acid may mean (i) a portion of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

As used herein, the term “wild type” sequence refers to a coding, non-coding or interface sequence is an allelic form of sequence that performs the natural or normal function for that sequence. Wild-type sequences include multiple allelic forms of a cognate sequence, for example, multiple alleles of a wild-type sequence may encode silent or conservative changes to the protein sequence that a coding sequence encodes.

As used herein the term “diagnosing” refers to classifying a pathology or a symptom, determining a severity of the pathology (grade or stage), monitoring pathology progression, forecasting an outcome of a pathology and/or prospects of recovery.

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one, which is expected to result in the best clinical outcome (e.g., complete cure of the pathology), or a more moderate one which may relieve symptoms of the pathology yet results in incomplete cure of the pathology. It will be appreciated that in certain cases the treatment regimen may be associated with some discomfort to the subject or adverse side effects (e.g., a damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

By “FLEXI RNA” is meant an excised linear intron RNA which is less than or equal to 300 nucleotides long. For example, the intron can be about 100, 150, 200, 250, or 300 nucleotides in length. The S′ end can be within 1, 2, or 3 nucleotides of an annotated 5′splice site, and the 3′ end can be within 1, 2, or 3 nucleotides of an annotated 3′ splice site. By “annotated splice site” is meant the site at which the intron is cleaved for excision (removal) by RNA splicing. It is noted that said annotation may have already occurred or may occur in the future. When both the 5′ and 3′ ends of the same intron RNA are found to be within 3 nucleotides of an annotated splice site, a full-length excised linear intron RNA has been identified.

By “intron RNA” is meant any RNA sequence that is removed by RNA splicing during maturation of the final RNA product. In other words, introns are non-coding regions of an RNA transcript, or the DNA encoding it, that are eliminated by splicing before translation. A “whole intron” refers to the entire segment which has been spliced, whereas an “intron fragment” refers to a portion of the whole intron, wherein the fragment is shorter than the whole intron by 1, 2, 3, 4, 5, 6, 7, 8, 9, 10, 11, 12, 13, 14, 15, 16, 17, 18, 19, 20, 21, 22, 23, 24, 25, 26, 27,28,29,30,31,32,33,34,35,36,37,38,39,40,41,42,43,44,45,46,47,48,49,50,51,52, 53,54,55,56,57,58,59,60,61,62,63,64,65,66,67,68,69, 70, 71, 72, 73, 74, 75, 76, 77, 78, 79,80, 81,82, 83,84, 85,86, 87, 88, 89,90,91,92,93,94,95,96,97, 98,99, 100,125,150, 175, 200, 225, 250, 275, or 300 nucleotides, or any amount greater, or in between, these amounts. “Intron fragment” can also refer a segment of an intron RNA that is 10%, 20%, 30%, 40%, 50%, 60%, 70%, 80%, or 90% or more (or any amount in between) identical to a full-length intron RNA For example, the intron can be 80% or more of the length of the intron from which it was derived. In another example, the intron fragment can be 60% or more but less than 80% of the length of the intron from which it was derived. Alternatively, it can be40% or more but less than 60% of the length of the intron from which it was derived; or 20% or more but less than 40% of the length of the intron from which it was derived; or less than 20% of the length of the intron from which it was derived, or any amount more, less, or in between these percentages.

The method of claim 93, wherein said fragment comprises a secondary structure, protein-biniling site, or sequence that renders it resistant to nuclease digestion.

### B. Methods of Identifying FLEXI-RNAS

Disclosed are methods and compositions related to determining one or more biomarkers in Full-Length Excised Intron RNAs (FLEXI RNAs). These biomarkers can be indicative of a specific characteristic, trait, disease, disorder or condition. The biomarker can be the presence or absence or a difference in the abundance of a FLEXI RNA in a biological sample from a subject exhibiting a trait, such as disease state, compared to that in a control sample from a subject that does not exhibit that trait. The biomarker can also be a single nucleotide change (such as an addition, subtraction, substitution, or post-transcriptional modification) in a FLEXI RNA when compared to a “wild type” or control biomarker, or it can be multiple differences in nucleotides in a given region, or across an entire FLEXI RNA 95. The biomarkers disclosed herein can occur in a fragment of an intron RNA. The biomarker can also be a difference in the ratio of a full-length intron RNA compared to one or more fragments of that RNA in a biological sample obtained from a subject exhibiting a trait compared to that in a control sample from a subject that does not exhibit that trait.

The FLEXI RNAs discovered by the methods disclosed herein can be useful in determining gene expression, alternative splicing, or differential stability. These characteristics can be used as biomarkers. The biomarkers disclosed herein can be predictive, diagnostic, prognostic, or can relate to drug interaction, drug response, or to a heritable condition. FLEXI RNAs found to be useful biomarkers for a specific trait can be incorporated into targeted RNA panels and kits by themselves or together with other RNA or non-RNA analytes for a variety of applications, including those using diagnostic, predictive, or prognostic biomarkers.

A diagnostic biomarker allows the detection of a disease, disorder or condition. A predictive biomarker allows predicting the response of the patient to a targeted therapy and so defining subpopulations of patients that are likely going to benefit from a specific therapy. A prognostic biomarker is a clinical or biological characteristic that provides information on the likely course of a disease, disorder or condition.

The FLEXI RNAs disclosed herein can be used to determine potential drug interaction or can be used to monitor the effects of drug interaction after they've been administered to a patient. The FLEXI RNAs disclosed herein can also be used to determine potential drug response in a patient, or to monitor the effects of a drug after it has been given. “Drug interaction” is a situation in which a substance affects the activity of a drug, i.e., the effects are increased or decreased, or they produce a new effect that neither produces on its own. However, interactions may also exist between drugs & foods (drug-food interactions), as well as drugs & herbs (drug-herb interactions). These may occur out of accidental misuse or due to lack of knowledge about the active ingredients involved in the relevant substances or the underlying molecular mechanisms. The FLEXI RNA biomarkers disclosed herein can be useful in determining what a subject's response to a certain drug or combination of drugs may be.

The FLEXI RNA biomarkers disclosed herein can also be used as markers of certain heritable traits, or phenotypic characteristics of a subject. Those of skill in the art will appreciate that such markers can be used to assess, on a genetic level, what those traits may be. This knowledge can be used, for example, in embryonic testing. The biomarkers can also be used to track disease progression in a subject.

Specifically, any disease, condition, trait or disorder that can be assessed through biomarker analysis can be detected using the methods disclosed herein. The disease or disorder includes without limitation a cancer, a premalignant condition, an inflammatory disease, an immune disease, an autoimmune disease or disorder, mental (psychological) disease or disorder, tissue damage, a cardiovascular disease or disorder, neurological disease or disorder, infectious disease or pain.

In some embodiments, when the biomarker is for cancer, the cancer comprises breast cancer, ovarian cancer, lung cancer, non-small cell lung cancer, small cell lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer, a glioblastoma, hepatocellular carcinoma, cervical cancer, endometrial cancer, head and neck cancer, esophageal cancer, gastrointestinal stromal tumor (GIST), renal cell carcinoma (RCC), gastric cancer, colorectal cancer (CRC), CRC Dukes B, CRC Dukes C-D, a hematological malignancy, B-cell chronic lymphocytic leukemia, B-cell lymphoma-DLBCL, B-cell lymphoma-DLBCL-germinal center-like, B-cell lymphoma-DLBCL-activated B-cell-like, or Burkitt's lymphoma.

The cancer can also comprise an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sezary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenstrom macroglobulinemia; or Wilm's tumor.

The premalignant condition can be without limitation actinic keratosis, atrophic gastritis, leukoplakia, erythroplasia, Lymphomatoid Granulomatosis, preleukemia, fibrosis, cervical dysplasia, uterine cervical dysplasia, xeroderma pigmentosum, Barrett's Esophagus, colorectal polyp, a transformative viral infection, HIV, HPV, or other growth or lesion at risk of becoming malignant.

In some embodiments, the autoimmune disease comprises inflammatory bowel disease (IBD), Crohn's disease (CD), ulcerative colitis (UC), pelvic inflammation, vasculitis, psoriasis, diabetes, autoimmune hepatitis, multiple sclerosis, myasthenia gravis, Type I diabetes, rheumatoid arthritis, psoriasis, systemic lupus erythematosis (SLE), Hashimoto's Thyroiditis, Grave's disease, Ankylosing Spondylitis Sjogrens Disease, CREST syndrome, Scleroderma, Rheumatic Disease, organ rejection, Primary Sclerosing Cholangitis, or sepsis.

In some embodiments, the cardiovascular disease comprises atherosclerosis, congestive heart failure, vulnerable plaque, stroke, ischemia, high blood pressure, stenosis, vessel occlusion, heart transplantation/rejection, or a thrombotic event.

The neurological disease detected, monitored, or prognosed with the methods disclosed herein can include, without limitation, Multiple Sclerosis (MS), Parkinson's Disease (PD), Alzheimer's Disease (AD), schizophrenia, bipolar disorder, depression, autism, Prion Disease, Pick's disease, dementia, Huntington disease (HD), Down's syndrome, cerebrovascular disease, Rasmussen's encephalitis, viral meningitis, neurospsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, or chronic fatigue syndrome.

In some embodiments, the pain comprises fibromyalgia, chronic neuropathic pain, or peripheral neuropathic pain. In other embodiments, the infectious disease comprises a bacterial infection, viral infection, yeast infection, Whipple's Disease, Prion Disease, cirrhosis, methicillin-resistant Staphylococcus aureus, HIV, HCV, hepatitis, syphilis, meningitis, malaria, tuberculosis, influenza.

The method of identifying biomarkers indicative of a specific characteristic, trait, disease, disorder or condition can comprise: a) obtaining FLEXI RNAs from one or more subjects with a specific characteristic, trait, disease, disorder or condition; b) determining the presence, absence, abundance sequence or sequences of FLEXI RNAs from said one or more subjects; c) comparing the presence, absence, abundance, sequence or sequences of said FLEXI RNAs from subjects with a specific characteristic, trait, disease, disorder or condition to the presence, absence, abundance, sequence or sequences of control FLEXI RNAs to determine differences; and d) determining which differences are indicative of a specific characteristic, trait, disease, disorder or condition, thereby identifying biomarkers for said specific characteristic, trait, disease, disorder or condition.

Said FLEXI RNAs can be identified, sequenced and their presence, absence, and abundance determined by RNA sequencing. Particularly useful for the identification of FLEXI RNAs are RNA sequencing methods that employ non-LTR-retroelement reverse transcriptases, such as group II intron-encoded reverse transcriptases, which have high processivity, strand displacement activity, fidelity, and template-switching activity that make it possible to obtain accurate, full-length, end-to-end reads of structured RNAs.

One, or more than one, biomarker in FLEXI-RNAs can be determined using the methods described herein. For example, at least 2, 3, 4, 5, 6, 7, 8, 9, 10, 11, 12, 13, 14, 15, 16, 17, 18, 19, 20, 25, 50, 75 or 100 or more different FLEXI RNA biomarkers can be determined. A single biomarker can be indicative of a disease, disorder, condition, trait, or characteristic, or more than one biomarker can be used to assess the same certain disease, disorder, condition, trait, or characteristic. Alternatively, two or more biomarkers can be used together in the same assay to determine more than one disease, disorder, condition, heritable trait, or characteristic at the same time. The two or more biomarkers can be present in the same gene, or in two or more different genes. For example, a panel of biomarkers can be used to assess one or more diseases, disorders, conditions, traits, or characteristic. The panel can include FLEXI RNAs discovered using the methods discussed herein. The panel can also comprise control FLEXI RNAs. Panels are described in more detail below.

In determining biomarkers using the methods disclosed herein, control FLEXI RNAs can be used. For example, the expression level of a biomarker can be compared to a control or reference, to determine the overexpression or underexpression (or upregulation or downregulation) of a biomarker in a sample. In some embodiments, the control or reference level comprises the amount of a same biomarker, such as a FLEXI RNA, in a control sample from a subject that does not have or exhibit the condition or disease. In another embodiment, the control of reference levels comprises that of a housekeeping marker whose level is minimally affected, if at all, in different biological settings such as diseased versus non-diseased states. In yet another embodiment, the control or reference level comprises that of the level of the same marker in the same subject but in a sample taken at a different time point. For example, two samples from the same patient can be taken at different time points to assess disease progression, to or monitor the effects of a treatment regime on the patient.

The methods disclosed herein of determining which differences are indicative of a specific characteristic, trait, disease, disorder or condition can be carried out via computer program The FLEXI RNAs disclosed herein can be specific for a cell or tissue type, and can be obtained from a variety of sources, including plasma. Further detail regarding computer programs and the methods disclosed herein follows.

Disclosed herein is a computer-implemented method for providing an evaluation for display, which evaluation is with respect to identifying one or more variations in one or more FLEXI RNAs that are associated with a specific characteristic, trait, disease, disorder or condition, comprising: a) obtaining sequence data from one or more FLEXI RNAs from subjects with and without a specific characteristic, trait, disease, disorder or condition; b) evaluating FLEXI RNA data from step a) using computer software executed on a computer to determine relevant biomarkers for a specific characteristic, trait, disease, disorder or condition, wherein said evaluation is algorithmically constructed and manipulated to detect patterns; and c) providing said evaluation for display on a computer generated report that identifies said one or more biomarkers in one or more FLEXI RNAs that are indicative of a specific characteristic, trait, disease, disorder or condition.

### C. Methods of Diagnosis/Prognosis and Treatment

Disclosed herein is a method of treating or preventing a disease or disorder in a subject, the method comprising: a) obtaining a sample from a subject; b) sequencing all or a portion of one or more Full-Length Excised Intron RNAs (FLEXI RNAs); c) analyzing sequence data from step b); d) determining that the subject has a disease or disorder based on results of step c); and e) treating or preventing the disease or disorder in the subject.

Further disclosed herein is a method of treating a subject based on disease prognosis for the subject, the method comprising: a) obtaining a sample from a subject; b) sequencing all or a portion of one or more Full-Length Excised Intron RNAs (FLEXI RNAs); c) analyzing sequence data from step b); d) determining disease prognosis for the subject based on results of step c); and e) treating the disease or disorder in the subject according to said prognosis.

Also disclosed herein is a method of determining potential drug interaction for a subject and treating the subject accordingly, the method comprising: a) obtaining a sample from a subject; b) sequencing all or a portion of one or more Full-Length Excised Intron RNAs (FLEXI RNAs); c) analyzing sequence data from step b) to determine potential drug interactions; and d) administering a drug or drugs based on the results of step c).

Further disclosed is a method of determining potential response to a drug in a subject and administering a drug based on results thereof, the method comprising: a) obtaining a sample from a subject; b) sequencing all or a portion of one or more Full-Length Excised Intron RNAs (FLEXI RNAs); c) analyzing sequence data from step b) to determine potential response to a drug; and d) administering a drug or drugs based on the results of step c).

Also disclosed herein is a method of tracking disease progression and/or response to treatment in a subject, and treating the subject accordingly, the method comprising: a) obtaining a sample from a subject; b) sequencing all or a portion of one or more Full-Length Excised Intron RNAs (FLEXI RNAs); c) analyzing sequence data from step b) to determine disease progression and/or treatment response; and d) treating the subject based on the results of step c). RNA can be isolated after the sample is obtained.

In all of the methods disclosed above, after obtaining a sample from the subject, the RNA can be isolated. This can be done by a variety of means known to those of skill in the art.

Said FLEXI RNAs can be analyzed using a variety of methods including, but not limited to microarray analysis or other hybridization-based assay, next-generation sequencing (NGS), reverse transcriptase polymerase chain reaction (RT-qPCR), Northern blot, serial analysis of gene expression (SAGE), immunoassay, and mass spectrometry. See, e.g., Draghici Data Analysis Tools for DNA Microarrays, Chapman and Hall/CRC, 2003; Simon et al. Design and Analysis of DNA Microarray Investigations, Springer, 2004; Real-Time PCR: Current Technology and Applications, Logan, Edwards, and Saunders eds., Caister Academic Press, 2009; Bustin A-Z of Quantitative PCR (IUL Biotechnology, No. 5), International University Line, 2004; Velculescu et al. (1995) Science 270: 484-487; Matsumura et al. (2005) Cell. Microbiol. 7: 11-18; Serial Analysis of Gene Expression (SAGE): Methods and Protocols (Methods in Molecular Biology), Humana Press, 2008, Hoffmann and Stroobant Mass Spectrometry: Principles and Applications, Third Edition, Wiley, 2007; herein incorporated by reference in their entireties.

In one embodiment, microarrays are used to measure the levels of biomarkers. An advantage of microarray analysis is that the expression of each of the biomarkers can be measured simultaneously, and microarrays can be specifically designed to provide a diagnostic expression profile for a particular disease or condition (e.g., cancer, regenerative medicine).

The specific disease that is diagnosed, detected, prognosed, or monitored can be, but is not limited to, cancer, an infectious disease, an autoimmune disease, tissue damage, or mental disease. Examples of these diseases and more are given above. More than one biomarker can be used in an assay, which is also described in detail above.

In certain embodiments, a panel of biomarkers is constructed based on the sequencing analysis of FLEXI RNAs using the methods disclosed herein. The panel can include a “control” or “reference.” Biomarker panels of any size can be used in the practice of the invention. Biomarker panels typically comprise at least 2 biomarkers, but can include 3, 4, 5, 6, 7, 8,9, 10, 15,20,25,30,35,40,45,50,55,60,65, 70, 75, 80, 85,90,95, 100, or more, including any number of biomarkers between. In certain embodiments, the invention includes a biomarker panel comprising at least 4, or at least 5, or at least 6, or at least 7, or at least 8, or at least 9, or at least 10 or more biomarkers.

Disclosed herein are methods of treating a subject based on the results of analyzing sequence data. For example, if the subject has been determined to have cancer, or the subject is prognosed with an aggressive or advanced stage of cancer, the subject can be treated with an anti-cancer therapy. The disclosed treatment regimens can include any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BICNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carne (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CV AD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride , Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R—CHOP, R—CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, , Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VelP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem, 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem, 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

Suitable carriers and their formulations are described in Remington: The Science and Practice of Pharmacy (19th ed.) ed. AR Gennaro, Mack Publishing Company, Easton, P A 1995. Typically, an appropriate amount of a pharmaceutically-acceptable salt is used in the formulation to render the formulation isotonic. Examples of the pharmaceutically acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable..

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### D. Computer-Implemented Methods

One, or more than one, FLEXI-RNA biomarkers can be determined using the computer-implemented methods described herein. For example, two or more FLEXI RNA biomarkers can be determined. When at least two biomarkers are present together, they can be indicative of a specific characteristic, trait, disease, disorder or condition. The two biomarkers can be present in the same, or in two or more different, genes. In determining biomarkers using the computer-implemented methods disclosed herein, control FLEXI RNAs from one or more subjects without the specific characteristic, trait, disease, disorder or condition can be used. The biomarkers disclosed herein for use in a computer-implemented method can be part of a panel. For example, the panel can include FLEXI RNAs discovered using the methods discussed herein. The panel can also comprise control FLEXI RNAs. The FLEXI RNAs disclosed herein for use in computer-implemented methods can be specific for a cell or tissue type, and can be obtained from a variety of sources, including plasma.

Further disclosed herein is a computer-implemented display for displaying the biomarkers identified in the computer-implemented methods disclosed herein.

In another embodiment, pattern recognition methods can be used. One example involves comparing biomarker expression profiles for various biomarkers to ascribe diagnoses/prognoses/predictions/outcomes. The expression profiles of each of the biomarkers is fixed in a medium such as a computer readable medium.

In one example, a table can be established into which the range of signals (e.g., intensity measurements) indicative of disease or physiological state is input. Actual patient data can then be compared to the values in the table to determine whether the patient samples are normal, benign, diseased, or represent a specific physiological state, for example. In a more sophisticated embodiment, patterns of the expression signals (e.g., fluorescent intensity) are recorded digitally or graphically. In the example of RNA expression patterns from the biomarker portfolios used in conjunction with patient samples are then compared to the expression patterns. Pattern comparison software can then be used to determine whether the patient samples have a pattern indicative of the disease, a given prognosis, a pattern that indicates likeliness to respond to therapy, or a pattern that is indicative of a particular physiological state. The expression profiles of the samples are then compared to the portfolio of a control. If the sample expression patterns are consistent with the expression pattern(s) for disease, prognosis, or therapy-related response then (in the absence of countervailing medical considerations) the patient is diagnosed as meeting the conditions that relate to these various circumstances. If the sample expression patterns are consistent with the expression pattern derived from the normal/control vesicle population then the patient is diagnosed negative for these conditions.

In another exemplary embodiment, a method for establishing biomarker expression portfolios is through the use of optimization algorithms such as the mean variance algorithm widely used in establishing stock portfolios. This method is described in detail in the U.S. Application Publication No. 2003/0194734, incorporated herein by reference. Alternatively, measured DNA alterations, changes in mRNA, protein, or metabolites to phenotypic readouts of efficacy and toxicity may be modeled and analyzed using algorithms, systems and methods described in U.S. Pat. Nos. 7,089,168, 7,415,359 and U.S. Application Publication Nos. 20080208784, 20040243354, or 20040088116, each of which is herein incorporated by reference in its entirety.

An exemplary process of biosignature portfolio selection (a combination of biomarkers) and characterization of an unknown is summarized as follows (see U.S. Pat. No. 9,128,101 for reference):
(1) Choose baseline class.
(2) Calculate mean, and standard deviation of each biomarker for baseline class samples.
(3) Calculate (X*Standard Deviation+Mean) for each biomarker. This is the baseline reading from which all other samples will be compared. X is a stringency variable with higher values of X being more stringent than lower.
(4) Calculate ratio between each experimental sample versus baseline reading calculated in step 3.
(5) Transform ratios such that ratios less than 1 are negative (e.g. using Log base 10).
(6) These transformed ratios are used as inputs in place of the asset returns that are normally used in the software application.
(7) The software will plot the efficient frontier and return an optimized portfolio at any point along the efficient frontier.
(8) Choose a desired return or variance on the efficient frontier.
(9) Calculate the Portfolio's Value for each sample by summing the multiples of each gene's intensity value by the weight generated by the portfolio selection algorithm.
(10) Calculate a boundary value by adding the mean Biosignature Portfolio Value for Baseline groups to the multiple of Y and the Standard Deviation of the Baseline's Biosignature Portfolio Values. Values greater than this boundary value shall be classified as the Experimental Class.
(11) Optionally one can reiterate this process until best prediction.

The process of selecting a biosignature portfolio can also include the application of heuristic rules. Preferably, such rules are formulated based on biology and an understanding of the technology used to produce clinical results. More preferably, they are applied to output from the optimization method. For example, the mean variance method of biosignature portfolio selection can be applied to microarray data for a number of biomarkers differentially expressed in subjects with a specific disease.

Other statistical, mathematical and computational algorithms for the analysis of linear and non-linear feature subspaces, feature extraction and signal deconvolution in large scale datasets for diagnosis, prognosis and therapy selection and/or characterization of defined physiological states can be done using any combination of unsupervised analysis methods, including but not limited to: principal component analysis (PCA) and linear and non-linear independent component analysis (ICA); blind source separation, nongaussinity analysis, natural gradient maximum likelihood estimation; joint-approximate diagonalization; eigenmatrices; Gaussian radical basis function, kernel and polynominal kernel analysis sequential floating forward selection.

A computer system can be used to transmit data and results following analysis. The computer system can be understood as a logical apparatus that can read instructions from media and/or network port, which can optionally be connected to server having fixed media. The system can include a CPU, disk drive, optional input devices such as keyboard and/or mouse and optional monitor. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present invention can be transmitted over such networks or connections for reception and/or review by a party. The receiving party can be but is not limited to an individual, a health care provider or a health care manager. Thus, the information and data on a test result can be produced anywhere in the world and transmitted to a different location. For example, when an assay is conducted in a differing building, city, state, country, continent or offshore, the information and data on a test result may be generated and cast in a transmittable form as described above. The test result in a transmittable form thus can be imported to receiving party.

Accordingly, the present invention also encompasses a method for producing a transmittable form of information on the diagnosis/prognosis/prediction of one or more samples from an individual. The method comprises the steps of (1) determining a diagnosis, prognosis, prediction, or other information or the like from the samples according to methods of the invention; and (2) embodying the result of the determining step into a transmittable form The transmittable form is the product of the production method. In one embodiment, a computer-readable medium includes a medium suitable for transmission of a result of an analysis of a biological sample, such as biosignatures.

The computer system can be any workstation, telephone, desktop computer, laptop or notebook computer, netbook, ULTRABOOK tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. In some embodiments, the computing device may have different processors, operating systems, and input devices consistent with the device. The Samsung GALAXY smartphones, e.g., operate under the control of Android operating system developed by Google, Inc. GALAXY smartphones receive input via a touch interface (see, for example, U.S. Patent Application 2013/0268290A1).

### E. Assays and Kits

Disclosed herein is an assay comprising a panel of biomarkers, wherein said biomarkers are found in FLEXI RNAs, wherein said biomarkers are indicative of a specific characteristic, trait, disease, disorder or condition. These assays and kits can be in the form of a microarray, for example. Said assays and kits can also comprise multiplex RT-qPCR and targeted RNA-seq panels.

Quantitative reverse transcription PCR (RT-qPCR) can be done by a variety of methods known to those of skill in the art, including a one-step or two-step method. RNA is first transcribed into complementary DNA (cDNA) by reverse transcriptase from total RNA or messenger RNA (mRNA). The cDNA is then used as the template for the qPCR reaction. RT-qPCR can be used in a variety of applications including gene expression analysis, RNAi validation, microarray validation, pathogen detection, genetic testing, and disease research.

Targeted RNA-sequencing (RNA-Seq) is a highly accurate method for selecting and sequencing specific transcripts of interest. It offers both quantitative and qualitative information. Targeted RNA-Seq can be achieved via either enrichment or amplicon-based approaches, both of which enable gene expression analysis in a focused set of genes of interest. Enrichment assays also provide the ability to detect both known and novel gene fusion partners in many sample types.

Microarrays are prepared by selecting probes which comprise a polynucleotide sequence, and then immobilizing such probes to a solid support or surface. The polynucleotide sequences of the probes may also be synthesized nucleotide sequences, such as synthetic oligonucleotide sequences. For more examples of microarrays, see U.S. Pat. No. 9,062,351.

The kits disclosed herein may include at least one agent that specifically detects at least one FLEXI RNA biomarker. It may include an assay for detecting more than one biomarker. It can also include a container for holding a biological sample isolated from the subject, and, optionally, printed instructions for reacting the agent with the biological sample or a portion of the biological sample to detect the presence or amount of at least one FLEXI RNA biomarker in the biological sample. The agents may be packaged in separate containers. The kit may further comprise one or more control reference samples and reagents for detection of biomarkers as described herein.

### F. Examples

The following examples are put forth so as to provide those of ordinary skill in the art with a complete disclosure and description of how the compounds, compositions, articles, devices and/or methods claimed herein are made and evaluated, are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Full-length Excised Intron RNAs (FLEXI RNAS) as Disease Biomarkers

### a) Summary

By using thermostable group II intron reverse transcriptase sequencing (TGIRT-seq), thousands of short, full-length excised intron RNAs (FLEXI RNAs; intron RNAs 300 nt with 5′ and 3′ ends within 3 nts of annotated splice sites) were identified in unfragmented (i.e., non-chemically fragmented) RNA preparations from human cells, tissues, and plasma. Most FLEXI RNAs are cell- or tissue-type specific, presumably reflecting differences in host gene transcription, alternative splicing, or differential stability of the FLEXI RNAs. FLEXI RNAS and the genes encoding them showed hundreds to thousands of readily detectable differences in matched healthy and breast cancer tissues from two patients with different breast cancer subtypes and the human breast cancer cell line MDA-MB-231. As many FLEXI RNAs are highly structured RNAs, their initial detection and characterization is done optimally by using TGIRT-seq, which has unprecedented ability to give accurate full-length, end-to-end sequence reads of structured RNAs. TGIRT-seq can be used to identify optimal combinations of FLEXI RNA and FLEXI RNA-encoding gene disease biomarkers, which could then be incorporated into targeted RNA panels that use different types of read outs, such as RT-qPCR, microarrays, other hybridization-based assays, or targeted RNA-seq. Such panels are more convenient and less costly than comprehensive RNA-seq and thus could facilitate diagnosis and routine monitoring of diseases progression and response to treatment. Because they are present in a large number of different genes and are related to changes in gene expression, FLEXI RNA biomarkers are applicable to all diseases as well as other a variety of other applications (e.g., monitoring response to environmental conditions, toxic chemicals, radiation, etc.). In addition to FLEXI RNAs, fragments or shorter segments of excised intron RNAs and the genes encoding them are other categories of potential biomarkers envisioned within the scope of this application.

TGIRT-seq datasets are summarized in Table 1. TGIRT-seq methods and applications are described in Nottingham et al., 2016; Qin et al., 2016; Shurtleff et al., 2017; and Xu et al., 2019.

Regarding Intron RNA fragments, analysis of commercial human plasma RNA pooled from healthy individuals identified sixteen peaks corresponding to intron RNA fragments that contain annotated RBP-binding sites and an another 15 such peaks were found among those mapping to long RNAs but lacking an annotated RBP-binding site. These 31 peaks ranged from 62-295 nucleotides in length. Paralleling findings for mRNA fragments in plasma, most of these intron peaks (25 peaks, 81%) could be folded by RNAfold into a stable secondary structure with predicted minimum free energies of less than −14.6 kcal/mol. The six intron peaks that could not be folded into stable secondary structures had other features that might contribute to their resistance to plasma nucleases. Three of these peaks consisted of AG-rich sequences or tandem repeats, including one with tandem AGAA repeats identified as an annotated binding site for TRA2A, a protein that helps regulate alternative splicing. Two others contained one arm of a long-inverted repeat sequence, whose complementary arm lies outside of the called peak and the remaining peak was a highly AU-rich RNA Thus, protection by bound proteins, stable RNA secondary structures, and unusual sequence features can contribute to the stability of these intron RNA fragments in the nuclease-rich environment of human plasma. Finally, it is noted that in addition to their biological and evolutionary interest, short full-length excised linear intron (FLEXI) RNAs and intron RNA fragments can be uniquely well-suited to serve as stable RNA biomarkers in cells and bodily fluids, whose expression is linked to that of numerous protein-coding genes. Intron RNA fragments are discussed in Yao et al., which is hereby incorporated by reference in its entirety for its teaching concerning intron fragments (Yao et al. Identification of Protein-Protected mRNA Fragments and Structured Excised Intron RNAs in Human Plasma by TGIRT-seq Peak Calling; eLife 2020;9:e60743).

### b) Materials and Methods for Example 1

DNA and RNA oligonucleotides. The DNA and RNA oligonucleotides used for TGIRT-seq on the Illumina sequencing platform are listed in Table 3. All oligonucleotides were purchased from Integrated DNA Technologies (IDT) in RNase-free HPLC-purified form. R2R oligonucleotides with equimolar A, C, G, and T 3′-overhang residues were hand-mixed prior to annealing to the R2 RNA oligonucleotide.

RNA preparations. Universal Human Reference RNA (UHRR) was purchased from Agilent (Cat #750500) and HeLa S3 RNA was purchased from ThermoFisher (Cat #QS0608). K-562 and HEK 293T cell RNAs were prepared from cultured cells. K-562 cells were cultured in IMDM+10% FBS medium, with −2 million cells used for RNA extraction. HEK 293T cells were cultured in DMEM high glucose pyruvate medium with −4 million cells used for RNA extraction. RNA was extracted from these cells by using a mirVana miRNA Isolation kit (Thermo Fisher, Cat #AM1560). MDA-MB-231 RNA was a gift from Morayma Temoche-Diaz and Randy Scheckman (University of California, Berkeley). RNAs from breast cancer patients frozen tissue samples were purchased from Origene (Cat #: CR562524, CR532030, CR543839, CR560540).

To remove residual DNA from RNA preparations, UHRR and HeLa S3 RNAs (1 μg) were treated with 20 U exonuclease I (Lucigen, Cat #X40520K) and 2 U Baseline-ZERO DNase (Lucigen, Cat #DB015K) in Baseline-ZERO DNase Buffer for 30 min at 37° C., and K562, MDA-MB-231 and HEK 293T RNAs (5 μg) were treated with 2 U TURBO DNase (Thermo Fisher, Cat #AM2239). After DNA removal, RNA was cleaned up with an RNA Clean & Concentrator kit (Zymo, Cat #R1314) with 8 volumes of ethanol (8X ethanol) added to maximize the recovery of small RNAs. The eluted RNAs were ribo-depleted by using the rRNA removal section of a TruSeq Stranded Total RNA Library Prep Human/Mouse/Rat kit (Illumina), with the supernatant from the magnetic-bead separation cleaned-up by using a Zymo RNA Clean & Concentrator kit with 8× ethanol. After checking RNA concentration and length by using a 2100 Bioanalyzer (Agilent) with an Agilent 6000 RNA pico chip, RNAs were aliquoted into −20 ng portions and stored at −80° C. until use.

Patient A and B matched breast cancer and healthy tissue pair RNAs (500 ng, Origene, Patient A: PR+, ER+, HER2−, CR562524/CR543839; Patient B: PR unknown, ER−, HER2−, CR560540/CR532030 were treated with 20 U exonuclease I (Lucigen, Cat #X40520K) and Baseline-ZERO DNase (Lucigen, Cat #DB015K) in Baseline-ZERO DNase Buffer for 30 min at 37° C. After clean up with an RNA Clean & Concentrator kit (Zymo, Cat #R1314) with 8 volumes of ethanol, the eluted RNA was ribo-depleted by using the rRNA removal section from a TruSeq Stranded Total RNA Library Prep Human/Mouse/Rat kit (Illumina). The supernatant from the magnetic-bead separation was cleaned-up by the Zymo RNA Clean & Concentrator kit (8× ethanol protocol). The size range and RNA concentration were verified by using a 2100 Bioanalyzer (Agilent) with an Agilent 6000 RNA pico chip, and the RNA was aliquoted into −20 ng portions for storage in −80° C.

For the preparation of chemically fragmented RNA samples, patient A and B RNAs (500 ng) were treated with 20 U exonuclease I (Lucigen, Cat #X40520K) and Baseline-ZERO DNase (Lucigen, Cat #DB015K) in 1× Baseline-ZERO DNase Buffer for 30 min at 37° C. After clean up with a RNA Clean & Concentrator kit (Zymo, Cat #R1314) with 8 volumes of ethanol (8× ethanol) added to the reaction to maximize the recovery of small RNAs, the eluted RNA was ribo-depleted by using the rRNA removal section from TruSeq Stranded Total RNA Library Prep Human/Mouse/Rat kit (Illumina). The supernatant from the magnetic-bead separation was cleaned-up by the Zymo RNA Clean & Concentrator kit using a two-fraction protocol that separates RNAs into long and short RNA fractions (200 nt cut-off).—50 ng of the long RNA fraction was fragmented to 70-100 nt by using an NEBNext Magnesium RNA Fragmentation Module (94° C. for 7 min; New England Biolabs). After clean-up by using a Zymo RNA Clean & Concentrator kit (8× ethanol protocol), the fragmented long RNAs were combined with the unfragmented short RNAs and treated with T4 polynucleotide kinase (Epicentre, Cat #P0503K) to remove 3′ phosphates that impede TGIRT template switching followed by clean-up by using a Zymo RNA Clean & Concentrator kit (8× ethanol protocol). The fragment size range and RNA concentration were verified by using a 2100 Bioanalyzer (Agilent) with an Agilent 6000 RNA pico chip, and the RNA was aliquoted into 4 ng portions for storage in −80° C.

TGIRT-seq. TGIRT-seq libraries were prepared as described using 20-50 ng of ribo-depleted unfragmented RNA or 4-10 ng of ribo-depleted chemically fragmented RNA The template-switching and reverse transcription reactions were done as described (Xu et al., 2019) with 1 μM TGIRT-111 (InGex) or Tel4c11EN RT (laboratory preparation) and 100 nM pre-annealed R2 RNA/R2R DNA in 20 μl ofreaction medium containing 200 or 450 mM NaCl, 5 mM MgCh, 20 mM Tris-HCl, pH 7.5 and 5 mM DTT. Reactions were set up with all components except dNTPs, pre-incubated for 30 min at room temperature, a step that increases the efficiency of TGIRT template-switching and reverse transcription, and then initiated by adding dNTPs (final concentrations 1 mM each of dATP, dCTP, dGTP, and dTTP). The reactions were incubated for 15 min at 60° C. and then terminated by adding 1 μl 5 M NaOH to degrade RNA and heating at 95° C. for 5 min followed by neutralization with 1 μl 5 M HCl and one round of MinElute column clean-up (Qiagen, Cat #28206). The RIR DNA adapter was adenylated by using an adenylation kit (New England Biolabs, Cat #E2610L) and then ligated to the 3′ end of the cDNA by using thermostable 5′ App DNA/RNA Ligase (New England Biolabs, Cat #0319L) for 2 hat 65° C. The ligated products were purified by using a MinElute Reaction Cleanup Kit and amplified by PCR with Phusion High-Fidelity DNA polymerase (Thermo Fisher Scientific, Cat #0531L): denaturation at 98° C. for 5 see followed by 12 cycles of 98° C. 5 see, 60° C. 10 see, 72° C. 15 see and then held at 4° C. The PCR products were cleaned up by using Agencourt AMPure XP beads (1.4× volume; Beckman Coulter) and sequenced on an Illumina NextSeq 500 instrument to obtain 2×75 nt paired-end reads.

Bioinformatics. All data analysis used combined TGIRT-seq datasets for different sample types listed in Table 1. Illurnina TruSeq adapters and PCR primer sequences were trimmed from the reads with Cutadapt v2.8 (Martin, 2011) (sequencing quality score cut-off at 20; p-value<0.01) and reads<15-nt after trimming were discarded. To minimize mismapping, a sequential mapping strategy was adopted. First, reads were mapped to the human mitochondrial genome (Ensembl GRCh38 Release 93) and Escherichia coli genome (Genebank: NC_000913) using HISAT2 v2.1.0 (Kim et al., 2019) with customized settings (−k 10—rfg 1,3—rdg 1,3—mp 4,2—no-mixed—no-discordant—no-spliced-alignment) to filter out reads derived from mitochondria or E. coli (denoted Pass 1). Unmapped read from Pass1 were then mapped to sncRNAs sequences (including human miRNA, tRNA, Y RNA, Vault RNA, 7SL and 7SK), 5S and 45S rRNA genes including the 2.2-kb 5S rRNA repeats from the 5S rRNA cluster on chromosome 1 (lq42, GeneBank: X12811) and the 43-kb 45S rRNA repeats that contained 5.8S, 18S and 28S rRNAs from clusters on chromosomes 13,14, 15,21, and 22 (GeneBank: U13369) using HISAT2 with the following settings (−k 20—rdg 1,3—rfg 1,3—mp 2,1—no-mixed—no-discordant—no-spliced-alignment—norc) (denoted Pass 2). Unmapped reads from Pass2 were then mapped to the human genome reference sequence (Ensembl GRCh38 Release 93) using HISAT2 with settings optimized for non-splicing mapping (−k 10—rdg 1,3—rfg 1,3—mp 4,2 —no-mixed—no-discordant—no-spliced-alignment) (denoted Pass 3) and splicing mapping (−k 10 —rdg 1,3—rfg 1,3—mp 4,2—no-mixed—no-discordant—dta) (denoted Pass 4). Finally, the remaining unmapped reads were mapped to Ensembl GRCh38 Release 93 by Bowtie 2 v2.2.5 (Langmead and Salzberg, 2012) using local alignment (with settings as:—k 10—rdg 1,3—rfg 1,3 —mp 4—ma 1—no-mixed—no-discordant—very-sensitive-local) to improve the mapping rate for reads containing post-transcriptionally added 5′ or 3′ nucleotides (poly(A) or poly(U)), short untrimmed adapter sequences, or non-templated nucleotides added to the 3′ end of the cDNAs by TGIRT enzymes (denoted Pass 5). For reads that map to multiple genomic loci with the same mapping score in passes 3 to 5, the alignment with the shortest distance between the two paired ends (i.e., the shortest read span) was selected. In the case of ties (i.e., reads with the same read span) for reads mapping to a chromosome and unpositioned contigs, the read was assigned to the main chromosome, and in other cases, the read was assigned randomly to one of the tied choices. Those filtered multiply mapped reads were then combined with uniquely mapped reads from Passes 3-5 by using Samtools v1.10 (Li et al., 2009) and intersected with gene annotations (Ensembl GRCh38 Release 93) with RNY5 gene and its 10 pseudogenes, which are not annotated in this release, added manually to generate the counts for individual features. Coverage of each feature was calculated by Bedtools v2.29.2 (Quinlan, 2014). To avoid miscounting reads with embedded sncRNAs that were not filtered out in Pass2 (snoRNA, snRNA, etc), reads were first intersected with sncRNA annotations and the remaining reads were then intersected with the annotations for protein-coding genes, lincRNAs, antisense, and other lncRNAs to get the correct read count for each annotated feature. Intron annotation were Extacted from Ensemble gene annotation using a customized script and filtered to remove introns>300 nt and duplicate annotations from mRNA isoforms. To calculate the coverage for FLEXI RNAs, mapped reads were intersected with intron annotations using Bedtools, and only read-pairs (Read1 and Read2) within 3 nucleotides of the annotated 5′—and 3′-splice sites were identified as being derived from full length excised intron RNAs.

Venn diagram of FLEXI RNAs from different cell type or conditions were plotted using Venn Diagram package v1.6.20 in R.

Density plots of length, CG content, minimum folding energy (MFE) and PhastCons scores of FLEXI RNAs were obtained using R (FIG. 2).

Coverage plots and read alignments were created by using Integrative Genomics Viewer v2.6.2 (IGV). Genes with>100 mapped reads were down sampled to 100 mapped reads in IGV for visualization.

TABLE 1
Summary of datasets for example 1.

| | Raw reads | | Trimmed reads | Mapped reads | Mapped to feature |
|---|---|---|---|---|---|
| RNA origin | (×106) | UMI | (×106) | (×106) | (×106) |
| HEK293T | 224.1 | N | 211.0 | 178.7 | 170.0 |
| | | | (94.2%) | (84.7%) | (95.2%) |
| Hela S3 | 851.0 | N | 803.3 | 768.4 | 705.6 |
| | | | (94.4%) | (95.7%) | (92.0%) |
| UHRR | 416.4 | N | 397.3 | 359.4 | 281.4 |
| | | | (95.4%) | (90.5%) | (79.4%) |
| K-562 | 206.4 | y | 54.6 | 45.8 | 42.9 |
| | | | (91.9%) | (84.0%) | (93.7%) |
| Plasma | 232.5 | y | 122.7 | 71.1 | 61.7 |
| | | | (91.3%) | (57.9%) | (87.2%) |
| MDA-MB-231 | 314.8 | N | 274.3 | 244.9 | 227.6 |
| | | | (87.1%) | (89.3%) | (92.9%) |
| Patient A Healthy | 327.7 | N | 317.4 | 283.3 | 258.9 |
| | | | (96.9%) | (89.3%) | (91.4%) |
| Patient A Cancer | 312.0 | N | 271.1 | 213.2 | 164.1 |
| | | | (86.9%) | (78.7%) | (77.0%) |
| Patient B Healthy | 296.1 | N | 282.7 | 249.9 | 232.2 |
| | | | (95.5%) | (88.4%) | (89.3%) |
| Patient B Cancer | 280.0 | N | 261.2 | 220.4 | 180.1 |
| | | | (93.3%) | (84.4%) | (81.7%) |
| Patient A Healthy | 55.7 | N | 52.7 | 50.5 | 33.2 |
| (Fragmented) | | | (94.7%) | (95.8%) | (60.1%) |
| Patient A Cancer | 61.9 | N | 59.8 | 57.0 | 40.5 |
| (Fragmented) | | | (96.7%) | (95.3%) | (68.8%) |
| Patient B Healthy | 39.6 | N | 35.1 | 33.1 | 22.0 |
| (Fragmented) | | | (88.5%) | (94.3%) | (57.2%) |
| Patient B Cancer | 58.4 | N | 56.9 | 54.1 | 47.1 |
| (Fragmented) | | | (97.5%) | (95.1%) | (85.5%) |

Two published datasets for HEK 293T cells (with/without YBX1 knockdown, SRX2887681 and SRX2887684, respectively) (Shurtleff et al., 2017).
Ten datasets for commercial universal human reference RNA (UHRR).
Ten datasets for commercial HeLa S3 cell RNA
Seven datasets for K-562 cells RNA with UMI in RIR adapter; cultures were vehicle controls for K-562 cell differentiation experiments.
Fifteen datasets from RNA extracted from commercial pooled plasma from healthy individuals with UMI in RIR adapter.
Five datasets for MDA-MB-231 cell RNA
Four datasets each for commercial matched healthy and cancer breast tissue RNA from patients A and B.
One dataset each for chemically fragmented RNAs from matched healthy/cancer tissue from patients A and B.
Abbreviations: UMI, unique molecular identifier for deconvolution of duplicate reads. N, no; Yyes.

TABLE 2
Total numbers ofFLEXI RNAs (2:5 reads) in unfragmented
RNAs samples from each cell or tissue type and numbers
that correspond to annotated mirtrons or agotrons.

| RNA | Total | Mirtron | Agotron | Agotron/Mirtron* |
|---|---|---|---|---|
| HEK293T | 1235 | 11 | 6 | 1 |
| Hela S3 | 1832 | 15 | 16 | 3 |
| UHRR | 1297 | 15 | 6 | 1 |
| K-562 | 201 | 5 | 3 | 0 |
| Plasma | 57 | 9 | 11 | 5 |
| MDA-MB-231 | 819 | 9 | 6 | 4 |
| Patient A Healthy | 175 | 14 | 13 | 5 |
| Patient A Cancer | 265 | 7 | 11 | 4 |
| Patient B Healthy | 113 | 8 | 7 | 6 |
| Patient B Cancer | 304 | 10 | 12 | 4 |

*Agotron/Mirtron indicates introns that were annotated as both an agotron and a mirtron.

TABLE 3
Oligonucleotides used in example 1.

| Name | Sequence and notes |
|---|---|
| NTC R2 | 5′-AGAUCGGAAGAGCACACGUCUGAACUCCAGUCAC/3SpC/ (SEQ ID NO: 1) |
| RNA | |
| NTTR2 | 5′-AAGAUCGGAAGAGCACACGUCUGAACUCCAGUCAC/3SpC/ (SEQ ID NO: 2) |
| RNA | |
| NTC R2R | 5′-GTGACTGGAGTTCAGACGTGTGCTCTTCCGATCTN-3′, where N is an equimolar of A, |
| DNA | C, G, T |
| | (obtained by hand mixing of individual oligonucleotides with A, C, G and T at |
| | their 3′ end). (SEQ ID NO: 3) |
| NTT R2R | 5′-GTGACTGGAGTTCAGACGTGTGCTCTTCCGATCTN-3′, where N is an equimolar of A, |
| DNA | C, G, T |
| | (obtained by hand mixing individual oligonucleotides with A, C, G and T at their |
| | 3′ end). (SEQ ID NO: 4) |
| RIR and | RIR DNA: 5′-/5Phos/GATCGTCGGACTGTAGAACTCTGAACGTGT AG/3SpC3/. ForUMI |
| UMI RIR | RIR DNAs, randomized nucleotides were added to the 5′ end. For 6N RlR, six |
| DNAs | machine-mixed randomized nucleotides were added to the 5′ end; for 8N RlR, eight |
| | machine-mixed randomized nucleotides were added to the 5′ end; and for ION RlR, |
| | ten machine-mixed randomized nucleotides were added to the 5′ end. The RIR and UMI |
| | RIR DNA oligonucleotides were adenylated. as described in Nottingham et al. 2016. |
| | (SEQ ID NO: 5) |
| Illumina | 5′-AATGATACGGCGACCACCGAGATCTACACGTTCAGAGTTCTACAGTCCGACGATC- |
| Multiplex | 3′ (SEQ ID NO: 6) |
| PCR | |
| primer | |
| Illumina | 5′CAAGCAGAAGACGGCATACGAGATBARCODE*GTGACTGGA |
| Illumina | GTTCAGACGTGTGCTCTTCCGATCT-3′, (SEQ ID NO: 7) where BARCODE correspond to |
| index PCR | one of the 6 nucleotide Illumina TmSeq barcode sequences. |
| primer | |

### 2. Example 2: Human cells Contain Myriad Excised Linear Introns with Potential Functions in Gene Regulation and as RNA Biomarkers

In this example, thermostable group II intron reverse transcriptase sequencing (TGIRT-seq) was used, which gives full-length end-to-end sequence reads of structured RNAs, to identify>8,500 short full-length excised linear intron (FLEXI) RNAs (:s; 300 nt) originating from>3,500 different genes in human cells and tissues. FLEXIs are distinguished from other introns by their accumulation as stable full-length linear RNAs. Subsets of the detected FLEXI correspond to pre-miRNAs of annotated mirtrons (introns that fold into a stem-loop structure and are processed by DICER into functional miRNAs) or agotrons (structured introns that bind AGO2 and function in a miRNA-like manner) and a few encode snoRNAs, but the vast majority had not been identified previously. FLEXI RNA profiles are cell-type specific, reflecting differences in transcription, alternative splicing, and intron RNA turnover, and comparisons of matched tumor and healthy tissues from breast cancer patients and cell lines revealed hundreds of differences in FLEXI RNA expression. About half the detected FLEXI RNAs contained a CLIP-seq identified binding site for one or more RNA-binding proteins. In addition to proteins that have RNA splicing- or miRNA-related functions, proteins that bind groups of 30 or more different FLEXI RNAs include transcription factors, chromatin remodeling proteins, and proteins involved in cellular stress responses and growth regulation, raising the possibility of previously unsuspected connections between intron RNAs and cellular regulatory pathways. These findings identify a large new class of human introns that can serve as RNA biomarkers.

### INTRODUCTION

Most protein-coding genes in eukaryotes consist of coding regions (exons) separated by non-coding regions (introns), which must be removed by RNA splicing to produce functional mRNAs. RNA splicing is performed by a large ribonucleoprotein complex, the spliceosome, which catalyzes transesterification reactions yielding ligated exons and an excised intron lariat RNA, whose 5′ end is linked to a branch-point nucleotide near its 3′ end by a 2′,5′-phosphodiester bond (Wilkinson et al. 2020). After splicing, this bond is typically hydrolyzed by a dedicated debranching enzyme (DBR1) to produce a linear intron RNA, which is rapidly degraded by cellular ribonucleases (Chapman and Boeke 1991). In a few cases, excised intron RNAs persist after excision, either as branched circular RNAs (lariats whose tails have been removed) or as unbranched linear RNAs, with some contributing to cellular or viral regulatory processes (Farrell et al. 1991; Kulesza and Shenk 2006; Gardner et al. 2012; Moss and Steitz 2013; Zhang et al. 2013; Pek et al. 2015; Talhouarne and Gall 2018; Morgan et al. 2019; Saini et al. 2019). The latter include a group of yeast introns that contributes to cell growth regulation and stress responses by accumulating as debranched linear RNAs that sequester spliceosomal proteins in stationary phase and other stress conditions (Morgan et al. 2019; Parenteau et al. 2019). Other examples are mirtrons, structured excised intron RNAs that are debranched by DBR1 and processed by DICER into functional miRNAs (Berezikov et al. 2007; Okamura et al. 2007; Ruby et al. 2007), and agotrons, structured excised linear intron RNAs that bind AGO2 and function directly to repress target mRNAs in a miRNA-like manner (Hansen et al. 2016).

Recently, while analyzing human plasma RNAs by Thermostable Group II Intron Reverse Transcriptase sequencing (TGIRT-seq), which gives full-length, end-to-end sequence reads of structured RNAs, 44 short(300 nt) full-length excised linear intron (FLEXI) RNAs were identified, subsets of which corresponded to annotated agotrons or pre-miRNAs of annotated mirtrons (denoted mirtron pre-miRNAs) (Yao et al. 2020). This discovery was followed up on by using TGIRT-seq to systematically search for FLEXI RNAs in human cell lines and tissues. About >8,500 different FLEXI RNAs were identified, many with stable predicted RNA secondary structures that would make them difficult to detect by other methods. By combining the newly obtained FLEXI RNA datasets with published CLIP-seq datasets, numerous intron RNA-protein interactions and potential connections to cellular regulatory pathways were identified that had not been seen previously. Finally, it was found that FLEXI RNA expression patterns were more discriminatory between cell types than were mRNAs from the corresponding host genes, showing utility as biomarkers for human diseases.

### Results

### Identification of FLEX! RNAs in Human Cells

A search of the human genome (Ensembl GRCh38 Release 93 annotations) identified 51,645 short introns (300 nt) in 12,020 different genes that could potentially give rise to FLEXI RNAs. To determine which of these short introns might give rise to FLEXI RNAS in biological samples, TGIRT-seq of ribodepleted, intact (i.e., non-chemically fragmented) human cellular RNAs was done, including Universal Human Reference RNA (UHRR; a mixture of total RNAs from ten human cell lines) and total cellular RNA from HEK-293T, K-562, and HeLa S3 cells (Table 4).

TGIRT-seq is particularly well-suited for the detection of excised linear intron RNAs. In the version of the method used here, the TGIRT enzyme initiates reverse transcription precisely at the 3′ nucleotide of a target RNA by template-switching from an RNA-seq adapter and then reverse transcribes to the 5′ end of the RNA, yielding a full-length intron cDNA to which a second RNA-seq adapter is ligated for minimal PCR amplification (see Methods). The high processivity and strand displacement activity of TGIRTs together with reverse transcription at elevated temperatures enable full-length end-to-end reads of highly structured RNAs (Katibah et al. 2014; Qin et al. 2016). In TGIRT-seq datasets of the ribodepleted non-chemically fragmented cellular RNAs, such as those obtained in this study, most of the reads correspond to full-length mature tRNAs and other structured sncRNAs (FIGS. 15 and 16). After ribodepletion, only a small percentage of the total reads (0.5-6.3%) corresponded to cellular or mitochondrial (Mt) rRNAs (FIG. 15). Additionally, because TGIRT-enzymes do not read through long stretches of poly(A), mRNA reads from protein-coding genes comprised a relatively low percentage of total reads (0.7-5.3%) and corresponded largely to nascent transcripts and non-or minimally polyadenylated mRNA sequences (FIG. 15).

To search for human FLEXIs in the TGIRT-seq datasets on intact cellular RNAs, the coordinates of all short introns (300 nt) were compiled in Ensembl GRCh38 Release 93 annotations into a BED file and searched for intersections with full-length excised linear intron RNAs, which were defined for these searches and all subsequent analyses as continuous intron reads whose 5′ and 3′ ends were within three nucleotides of annotated splice sites. For each sample type, the searches were done by using combined datasets obtained from multiple replicate libraries totaling 666 to 768 million mapped reads for the cellular RNA samples (Table 4). In addition to human cellular RNAs, we used this approach to search remapped datasets of human plasma RNAs from healthy individuals (Yao et al. 2020). We thus identified 8,144 different FLEXI RNAs represented by at least one read in any of the cellular or plasma RNA datasets. These FLEXI RNAs originated from 3,743 different protein-coding genes, lncRNA genes, or pseudogenes (collectively denoted FLEXI host genes; FIG. 7).

UpSet plots and pairwise scatter plots comparing different cell lines showed that both FLEXI RNA and FLEXI host gene expression patterns were cell type-specific (FIG. 7A and B). Notably, the scatter plots for FLEXI RNAs showed greater discrimination between cell types than did those for all transcripts from the corresponding host genes, with numerous FLEXIs of abundances up to 1 to 7 RPM detected in only one or the other of two compared cell types (FIG. 7B). Principal component analysis (PCA), as well as PCA-initialized t-SNE (Kobak and Berens 2019) and ZINB-WaVE (Risso et al. 2018), both of which are widely used for the analysis of single cell RNA-seq datasets with zero inflated counts, showed clustering of cell-type-specific FLEXI RNA profiles in 44 different replicates of the cellular RNA datasets obtained in this study (FIG. 17).

Density plots of the length distribution of all reads that mapped to introns in a combined dataset for the UHRR, K-562, HEK-293T and HeLa S3 RNA samples showed a peak at near 100% of the full intron length for the detected FLEXIs, whereas reads mapping to other short introns (300 nt) annotated in GRCh38 corresponded largely to heterogeneously sized fragments, as expected for the more typical situation of intron RNAs that turnover rapidly after RNA splicing (FIG. 7C, left panel). Similar patterns were seen in UHRR and the individual cell types for different subgroups of FLEXIs described further below, except for a subgroup of FLEXIs containing annotated binding sites for DICER, which showed additional peaks corresponding to discrete shorter intron RNA fragments, as expected for DICER cleavage (FIG. 18). These findings identify FLEXIs as a distinct class of short introns that are stable in cells as full-length linear RNA molecules.

### Sequence and Structural Characteristics of Detected FLEX! RNAs

Integrative Genomic Viewer (IGV) alignments showed that the FLEXIs detected by TGIRT-seq are full-length linear intron RNAs, with reads extending continuously from the 5′—to 3′-splice site even for highly structured FLEXIs and no stops or base substitutions that may show the presence of a branched nucleotide residue (FIG. 8A-C). Analysis of FLEXI RNA expression in different cell-types indicated that differences in FLEXI RNA abundance reflect differences in host gene transcription, alternative splicing, or stability of the excised intron RNAs, the latter suggested by differences in the relative abundance of non-alternatively spliced FLEXIs transcribed from the same gene (examples shown in FIG. 8D).

Most of the detected FLEXI RNAs had sequence characteristics of major U2-type spliceosomal introns (8,082, 98.7% with canonical GU-AG splice sites and 1.3% with GC-AG splice sites), with only 36 FLEXI RNAs having sequence characteristics of minor U12-type spliceosomal introns (34 with GU-AG and 2 with AU-AC splice sites), and 23 having non-canonical splice sites (e.g., AU-AG and AU-AU; FIG. 9A and FIG. 19) (Burset et al. 2000; Sheth et al. 2006). The identified FLEXI RNAs had a canonical branch-point (BP) consensus sequence (FIG. 9A) (Gao et al. 2008), suggesting that most if not all were excised as lariat RNAs and debranched after splicing, as found for mirtron pre-miRNAs (Okamura et al. 2007; Ruby et al. 2007).

In a previous analysis of human plasma RNAs, 44 different FLEXI RNAs were identified, of which 13 corresponded to annotated agotrons and 10 corresponded to pre-miRNAs of annotated mirtrons, with 7 annotated as both an agotron or mirtron (Yao et al. 2020). Of the>8,000 different FLEXI RNAs detected here in the human cellular RNA and remapped plasma RNA datasets, 65 corresponded to an annotated agotron (Hansen et al. 2016) and 114 corresponded to a pre-miRNA for an annotated mirtron (FIG. 9B and C) (Berezikov et al. 2007). Notably, the proportion of FLEXI RNAs corresponding to annotated agotrons or mirtron pre-mRNAs in plasma (22.8%) was considerably higher than that in the cellular RNA preparations (1.7-2.6%; FIG. 9C), possibly reflecting preferential cellular export or greater stability of these RNAs in plasma (Yao et al. 2020). A small number of FLEXI RNAs found in cells but not plasma (43, 0.5% of the total) encode snoRNAs, all of which were also detected as mature snoRNAs in the same RNA samples (FIG. 9C).

Analysis of other characteristics showed that the 224 FLEXI RNAs detected in human plasma were a relatively homogeneous subset with peaks at 90-nt length, 70% GC content, and −40 kcal/mole minimum free energy (MFE; G) for the most stable RNA secondary structure predicted by RNAfold (FIG. 7C). By comparison, the FLEXI RNAs detected in cells were more heterogeneous, with similar peaks but larger shoulders extending to longer lengths, lower GC contents, and less stable predicted secondary structures (−25 kcal/mole; FIG. 7C). The more homogeneous subset of FLEXI RNAs found in plasma could reflect preferential export or greater resistance to plasma RNases of shorter, more stably structured FLEXI RNAs.

### Abundance of FLEXIs in Cellular RNA Samples

FIG. 7D shows density plots of the abundance (RPMs) of different categories of FLEXIs in the different cellular RNA samples compared to those of sncRNAs spanning a range of different cellular abundances in the same samples (Table 5). The large numbers of newly identified FLEXIs (denoted All other FLEXIs) showed two major peaks: one at −0.001 RPM and the other between 0.002 and 0.1 RPM with a tail extending to 1.3-6.9 RPM in the different cellular RNA samples. In HeLa S3 cells, the peak between 0.01 and 0.1 RPM was predominant with only small peaks at lower abundances. In each of the cellular RNA samples, the FLEXIs previously annotated as agotrons, mirtrons, or containing embedded snoRNAs overlapped the peak of more abundant FLEXI RNAs. The abundances of most FLEXIs overlapped the lower end of the abundance distribution for snoRNAs in the same samples. By using sncRNAs of known cellular abundance to produce a linear regression model for the relationship between log lotrans formed copy number per cell and RPM values in the TGIRT-seq datasets, it was estimated that the most abundant FLEXIs (1 RPM) may be present at 1-2×10³ molecules per cell and that substantial numbers of FLEXIs with RPMs 0.01 RPM (20-87% in different cellular RNA samples) may be present at 150-187 copies per cell (FIG. 20).

FLEXIs Exhibit Different Degrees of Evolutionary Conservation and Highly Conserved FLEXIs are Associated with a Distinct Set of RNA-Binding Proteins

FIG. 7C right panel shows density plots of phastCons scores for all FLEXIs detected in a combined dataset for the human cellular RNA samples, with those corresponding to mirtrons, agotrons, and snoRNAs again split out as separate categories from all other FLEXIs. Most FLEXIs, including those corresponding to mirtron pre-miRNAs or agotrons, had low phastCons scores with peaks at 0.06-0.09 compared to 0.02 for other annotated short introns in GRCh38 and with tails extending to higher phastCons scores. As might be expected, FLEXIs encoding snoRNAs had higher phastCons scores (four at 0.5) than did other FLEXIs (FIG. 7C, right panel, yellow line). The low phastCons scores for most FLEXIs, including those with biological functions as agotrons or mirtron pre-miRNAs, indicates that they were acquired recently in the human lineage or have undergone rapid sequence divergence.

Five percent (399) of the detected FLEXIs that were not annotated as mirtrons, agotrons or encoding snoRNAs, had phastCons scores>0.47 and 2% (159) had phastCons scores>0.74), possibly reflecting an evolutionarily conserved sequence-dependent function. At the high end of the spectrum, 44 FLEXI RNAs had phastCons scores(0.99). Forty-one of these highly conserved FLEXIs were within protein-coding sequences and 37 were known to be alternatively spliced to generate different protein isoforms, with 26 sharing 5′—or 3′-splice sites with a longer intron and 16 containing in-frame protein-coding sequences that would be expressed if the intron was retained in a mRNA (examples in HNRNPL, HNRNPM, and FXRJ; UCSC genome browser). A FLEXI in the human EIFJ gene with phastCons score=1.00 resulted from acquisition of a 3′-splice site in its highly conserved 3′ UTR and is spliced to encode a novel human-specific EIFI isoform (chrl7:41,690,818-41,690,902) (Kim et al. 2020).

A search of CLIP-seq datasets (Hafner et al. 2010; Rybak-Wolf et al. 2014; Van Nostrand et al. 2016) identified a group of RNA-binding proteins (RBPs), whose binding sites were significantly enriched (p:s; 0.05 calculated by Fisher's exact test) in highly conserved FLEXIs (phastCons scores 0.99), including alternative splicing regulators (KHSRP, TIAL1, TIA1, PCBP2), extrinsic splicing factors (SFRS1, U2AF1, U2AF2), and a number of protein with no known RNA splicing- or miRNA-related function described further below (FIG. 9D). By contrast, annotated binding sites for core spliceosomal proteins (AQR, BUD13, EFTUD2, PRPF8, SF3B4) were under-represented in these highly conserved FLEXI RNAs (FIG. 9D). FLEX! RNAs contain experimentally identified binding sites for a variety of RNA-binding proteins

The finding above that highly conserved FLEXIs are enriched in CLIP-seq identified binding sites for a distinct set of RBPs prompted a comprehensive search for RBPs associated with different FLEXI RNAs in high confidence eCLIP (Van Nostrand et al. 2016), DICER PAR-CLIP (Rybak-Wolf et al. 2014), and AGO1-4 PAR-CLIP (Hafner et al. 2010) datasets. It was found that more than half of the detected FLEXI RNAs (4,505; 55%) contained an experimentally identified binding site for one or more of 126 different RBPs (FIG. 10, FIG. 21). These 126 RBPs included spliceosome components and proteins that function in RNA splicing regulation; DICER, AGOI-4 and other proteins that function in the processing or function of miRNAs; and a surprising number of proteins whose primary functions are unrelated to RNA splicing or miRNAs. Notably, 121 of the identified RBPs had CLIP-seq-identified binding sites in multiple different FLEXI RNAs (FIG. 21), with 53 RBPs having CLIP-seq-identified binding sites in 30 or more different FLEXI RNAs (FIG. 10A).

Overall, compared to longer introns>300 nt or all RBP-binding sites in the CLIP-seq datasets, the detected FLEXI RNAs were significantly enriched (p:s; 0.05 calculated by Fisher's exact test) in CLIP-seq-identified binding sites for six spliceosomal proteins (AQR. BUD13, EFTUD2, PPIG, PRPF8, SF3B4), with these proteins found associated with 740 to 1,922 different FLEXI RNAs (FIG. 10). The enrichment of CLIP-seq identified binding sites for this set of spliceosomal proteins in this large group of FLEXI RNAs indicates that they may dissociate more slowly from spliceosomal complexes than do longer introns.

Many of the detected FLEXIs also contained CLIP-seq-identified binding sites for proteins with miRNA-related functions, with 250 containing a binding site for AGO1-4, 308 containing a binding site for DICER, and 66 containing binding sites for both AGO1-4 and DICER (FIG. 10A). However, only 23 of the 250 FLEXI RNAs identified as a binding site for AGO1-4 in the AGO1-4 PAR-CLIP dataset corresponded to an annotated agotron (Hansen et al. 2016) and only 44 of the 308 FLEXIs identified as binding site for DICER in the DICER PAR-CLIP dataset corresponded to a pre-miRNA for an annotated mirtron (FIG. 9C) (Wen et al. 2015). The large numbers of additional FLEXIs containing AGO1-4 or DICER binding sites could be unannotated agotrons or mirtrons. Alternatively, they could be processed by DICER into other types of short regulatory RNAs, function as sponges for AGO1-4 and DICER, or affect the subcellular localization of these proteins, as found recently for a circular RNA linked to aberrant nuclear localization of DICER in glioblastoma (Bronisz et al. 2020). As noted previously, the FLEXI RNAs with annotated DICER-binding sites differed from other FLEXIs in showing discrete size classes of relatively abundant shorter RNA fragments, as expected for DICER cleavage (FIG. 18).

Surprisingly, 23 RBPs that bind 30 to 365 different FLEXI RNAs have no known RNA splicing- or miRNA-related function (FIG. 10A, protein names in black; Table 6). They instead function in a variety of other cellular processes, including regulation of transcription, apoptosis, stress responses, cellular growth regulation, and histone assembly and disassembly (summarized in Table 6), potentially linking FLEXI RNA binding to the regulation of these processes. In general, the binding of these protein to FLEXI RNAs can contribute to the regulation of cellular processes by regulating the splicing and expression of the FLEX host genes, by forming an RNP complex that functions directly in the process or its regulation; or by changing the intracellular localization or level or free protein, particularly for those proteins that bind large numbers of different FLEXI RNAs.

Subsets of FLEXIs bind RBPs that perform specialized biological Junctions

Although the majority of FLEXI RNAs have annotated binding sites for spliceosomal proteins, the findings above that highly conserved FLEXIs were enriched in CLIP-seq-identified binding sites for other types of proteins and under-represented in binding sites for spliceosomal proteins (FIG. 9D) suggested that there could be different classes of FLEXIs that bind different RBPs, possibly in order to perform specialized biological functions. Precedents for the latter are agotrons and mirtrons, which presumably dissociate from the spliceosome and preferentially bind AGO1-4 to downregulate mRNAs or DICER to function as miRNA precursors (Berezikov et al. 2007; Hansen et al. 2016).

To search for such FLEXIs, RBPs with no known RNA splicing- or miRNA-related function that bind 30 or more different FLEXIs were the focus and examined in UpSet plots to assess the extent to which these RBPs are associated with FLEXIs that lack CLIP-seq-identified binding sites for the five most ubiquitous core spliceosomal proteins (PRPF8, SF3B4, AQR, EFTUD2, and BUD13), which collectively bind thousands of other FLEXI RNAs (FIG. 10A). Using agotrons and mirtrons as standards for FLEXI RNAs with specialized biological functions, it was found that 51% of the FLEXIs with a CLIP-seq-identified binding site for AGO1-4 and 44% of those with a CLIP-seq-identified binding site for DICER lacked annotated binding sites for any of the five ubiquitous spliceosomal proteins (FIG. 11A and B). Similar UpSet plots for the 23 RBPs that bind 30 or more different FLEXIs but have no known RNA splicing- or miRNA-related function identified 16 for which substantial proportions (29-55%) of the bound FLEXIs lacked annotated binding sites for any of the five spliceosomal proteins (examples shown in FIG. 11C-H; others listed in the legend of FIG. 11 and indicated by at in FIG. 12 below). These findings show that after splicing, some groups of FLEXIs may preferentially bind other non-splicing related RBPs with a variety of cellular functions.

FLEXIs that Bind the Same REP Originate from Host Genes with Related Biological Junctions

To further explore the biological significance of these FLEXI RNA-RBP interactions, hierarchical clustering was performed based on GO terms for the host genes encoding FLEXI RNAs bound by the same RBP. Focusing again on those RBPs that bind 30 or more different FLEXI RNAs, we identified five major clusters of FLEXIs whose host genes showed significant enrichment for different sets of biological processes (FIG. 12). By contrast, a control group consisting of the 3,639 detected FLEXI RNAs that did not contain an annotated RBP-binding site in the CLIP-seq datasets originated from host genes that showed no similar enrichment for GO terms associated with biological processes (FIG. 12, right lane), nor did randomly sampled subsets of host genes for all FLEXIs, FLEXIs that did not contain an annotated RBP-binding site, all human genes, or all human genes than contain short introns (:s; 300 nt) over a range of different randomly sampled pool sizes (FIG. 22). These controls indicate that the GO term enrichment for FLEXIs bound by different RBPs is not merely a byproduct of random sampling of the genes encoding FLEXIs. Collectively, these findings suggest that the host genes encoding FLEXI RNAs bound by the same RBP have related biological functions and thus might be coordinately regulated to produce different subsets of FLEXI RNPs.

### FLEX! RNAs May Junction in Diverse Cellular Regulatory Pathways

The GO term clustering and biological functions of the host genes encoding FLEXI RNAs bound by different RNPs identify previously unsuspected interactions and connections to cellular regulatory pathways. Cluster I comprised of FLEXI RNAs that bind the five ubiquitous core spliceosomal proteins (SF3B4, BUD13, EFTUD2, AQR, PRPF8) plus AGO1-4 originated from host genes associated with the widest variety of biological processes, whereas clusters II to V were comprised of FLEXI RNAs whose host genes were associated with different subsets of these processes. The host genes for FLEXI RNAs bound by the RBPs in cluster II were enriched for GO terms involved with rRNA processing, translation, and mRNA splicing, while those in cluster III were enriched for a smaller set of GO terms involved with rRNA processing and translation.

Notably, cluster III includes three RBPs (DKC1, NOLC1, and AATF; denoted with § in FIG. 12) that have annotated binding sites in overlapping sets of FLEXIs that also contained annotated binding sites for the five core spliceosomal proteins (FIG. 23). The FLEXI RNAs bound by these RBPs were distinguished by relatively low GC content (peak at 30-40% GC) and above average phastCons scores (peaks at 0.3 to 0.4; FIG. 24A), and upon further examination were found to include 41 of the 43 FLEXIs that encode snoRNAs. DKC1 (dyskerin) and NOLC1 (nucleolar and coiled-body phosphoprotein 1) are components of snoRNPs that bind intronic snoRNA sequences co-transcriptionally to delineate these regions for snoRNA processing (Kufel and Grzechnik 2019), possibly accounting for the occurrence of CLIP-seq identified spliceosomal protein binding sites in the same FLEXIs (FIG. 23). DKC1 also stabilizes telomerase RNA (MacNeil et al. 2019), and NOLC1 interacts with TRF2 (Telomeric Repeat-Binding Factor 2) to mediate its trafficking between the nucleolus and nucleus (Yuan et al. 2017). The third protein, AATF (Apoptosis Antagonizing Transcription Factor), is a Pol II-interacting protein that regulates the function of the p53 and Rb oncogenes and is over produced in many cancers to inhibit apoptosis (Iezzi and Fanciulli 2015; Kaiser et al. 2019). A recent study found that AATF binds 45S precursor rRNA, as well as mRNAs encoding ribosome biogenesis factors and both H/ACA- and C/D-box snoRNAs, leading to the hypothesis that AATF involvement in ribosome biogenesis might be linked to its role in apoptosis (Kaiser et al. 2019). However, 11 of the 34 FLEXIs bound by AATF are short introns that neither encode snoRNAs nor are in genes related to ribosome biogenesis, and AATF also binds to 925 long introns (>300 nt) of which only 295 encode snoRNAs, suggesting a broader role for AATF linked to RNA splicing or intron binding. Cluster III also includes RPS3, which has been implicated in regulating transcription, DNA damage response, and apoptosis (Gao and Hardwidge 2011); DDX3X, a DEAD-box RNA helicase with functions in regulating stress granule formation and apoptosis (Schroder 2010; Hilliker et al. 2011); and YBX3, a homolog of YBX1, a low specificity RBP that plays a role in regulating stress granule assembly, sorting small non-coding RNAs into extracellular vesicles, and a variety of other cellular processes (Somasekharan et al. 2015; Shurtleff et al. 2017).

The host genes for the FLEXI RNAs in cluster IV are enriched in many of the same GO terms related to RNA splicing as cluster II plus additional GO terms related to transcription and chromatin (FIG. 12). Surprisingly, 8 of the 12 RBPs that comprise this cluster corresponded to those identified above (FIG. 9D) as binding FLEXI RNAs with very high phastCons scores(0.99; BCLAFI, GRWD1, SRSF1, TIA1, UCHL5, U2AF1, U2AF2, and ZNF622; denoted with asterisks in FIG. 12), although these proteins also bind many additional FLEXIs with lower phastCons scores (FIG. 24B). Five of these proteins as well as TRA2 in cluster IV bind FLEXIs with significantly lower GC content than other FLEXIs (FIG. 24B). Four of the proteins that bind highly conserved FLEXIs (TIA1, SRSF1, U2AF1, and U2AF2) function in the regulation of alternative splicing, as does TRA2A and possibly PPIG, which is also found in this cluster, potentially providing examples of subsets of FLEXI RNPs that result from alternative splicing regulation. TIA1 also plays a key role in stress granule formation (Kedersha et al. 1999); GRWD1 is a histone-binding protein that regulates chromatin dynamics (Sugimoto et al. 2015); and BCLAFI (BCL2-2-associated transcriptional factor) and ZNF622 are positive regulators of apoptosis (Vohhodina et al. 2017), increasing to five the number of FLEXI RNA-binding proteins connected to this process (see above).

194. The host genes for the FLEXI RNAs in cluster V are enriched in only a few GO terms for each RBP. Four of these proteins function in splicing regulation (LSM1 1, PCBP2, RBFOX1, and GPKOW) and three others are notable regulatory proteins: IGF2BP1 (insulin-like growth factor 2 mRNA-binding protein!), which functions in cell cycle regulation (Muller et al. 2020); G3BP1 (Ras GTPase-activating protein binding protein 1), a helicase that plays an essential role in innate immunity, functions in stress granule assembly, is associated with cellular senescence, and regulates important signaling pathways (Zhang et al. 2019; Biermann et al. 2020; Omer et al. 2020); and PABPN1 (polyadenylate-binding protein 2), a crucial player in double-strand-break repair (Gavish-Izakson et al. 2018). Three of these proteins (IGF2BP1, LSM1 1, and G3BP1) were among those binding to substantial subsets of FLEXIs that lacked annotated binding sites for the five core spliceosomal proteins (see above). Collectively, these findings show that the binding of non-spliceosomal RBPs to different subsets of FLEXIs can be linked to multiple cellular regulatory pathways.

### FLEX! RNAs as Potential Cancer Biomarkers

The cell- and tissue-specific expression patterns of FLEXI RNAs showed that they can be useful as biomarkers to distinguish normal and abnormal cellular states. To test this, FLEXI RNAs and FLEXI host genes in matched tumor and neighboring healthy tissue from two breast cancer patients (patients A and B; PR+, ER+, HER2—and PR unknown, ER−, HER2−, respectively) and two breast cancer cell lines (MDA-MB-231 and MCF7) were examined. UpSet plots showed hundreds of differences in FLEXI RNAs and FLEXI host genes between the cancer and healthy samples (FIG. 13A for FLEXIs detected at 0.01 RPM and FIG. 25 for FLEXI RNAs detected at 1 read). The discriminatory ability of FLEXIs was also evident in scatter plots comparing the FLEXI RNAs detected in the matched healthy and tumor samples from patients A and B, which showed a wider spectrum of differences than did those for mRNAs from the same host genes quantitated in chemically fragmented RNA preparations (FIG. 13B). The scatter plots identified multiple candidate FLEXI RNA biomarkers, including 18 and 16 in patients A and B, respectively, that were detected at relatively high abundance (0.05-0.16 RPM) and in at least two replicate libraries from the cancer patient, but not detected in the matched healthy tissue (dots in FIG. 13B, genes listed to the right).

GO enrichment analysis of FLEXI RNA host genes in the four cancer samples but not healthy tissues showed significant enrichment (p 0.05) of hallmark gene sets (Liberzon et al. 2015) that may be disregulated in many cancers (e.g., glycolysis, G2M checkpoint, UV response up, and PI3K/AKT/MTOR signaling; FIG. 13C). Gene sets that were significantly enriched in one or more of the cancer samples but not in the healthy controls included mitotic spindle, MYC targets V1 and V2, estrogen response early and late, androgen response, oxidative phosphorylation, mTORC1 signaling, apical junction, and cholesterol homeostasis (FIG. 13C).

Only a small number of the potential FLEXI RNA biomarkers identified in the UpSet and scatter plots in FIG. 13 corresponded to previously identified oncogenes (names with asterisks in FIG. 13A and B). This reflects a combination of factors, including that some prominent oncogenes (e.g., CD24, ERAS, and MYC) as well as hormone receptors genes (ERBB2, ESRJ, and PR) do not encode FLEXIs; that FLEXI RNA abundance is dictated by alternative splicing and intron RNA turnover in addition to transcription; and that those FLEXI RNAs that best discriminate between cancer and healthy samples arise from genes that are strongly up or downregulated in response to oncogenesis but are not oncogenes that drive this process.

To directly examine the relationship between FLEXI RNAs and oncogene expression, FLEXI RNAs from known oncogenes were identified (n=803) (Liu et al. 2017) that were 2-fold up- or downregulated in any of the cancer samples. UpSet plots identified 169 FLEXI RNAs from known oncogenes that were up upregulated in any of the cancer samples compared to the healthy controls, with 13 to 60 upregulated in only one of the cancer samples and 5 upregulated in all four cancer samples (FIG. 14A). Another 81 FLEXI RNAs from known oncogenes were downregulated by 2-fold in any of the cancer samples compared to healthy controls, with 1 to 29 downregulated in only one of the cancer samples and 4 downregulated in all four cancer samples (FIG. 14B). Similar patterns were seen in UpSet plots for up— and downregulated tumor suppressor genes (n=1,217) (Zhao et al. 2016) (FIG. 14C and D).

The up and down patterns for FLEXI RNAs from both oncogenes and tumor suppressor genes again reflect that FLEXI RNA abundance is dictated by factors other than transcription (FIG. 8). The FASN (fatty acid synthase) gene, for example, contains 8 FLEXIs that were upregulated and 5 that were down regulated in the cancer samples (e.g., FASN-131 and FASN81 in MCF7 cells and FASN-311 and FASN-261 in patient B; FASNFLEXIs highlighted in red in FIG. 14A and B). This situation does not preclude these introns from serving as a biomarker for a specific cancer, so long as they are found to be reproducibly up or downregulated in that cancer.

Notably, the RBP-binding sites enriched in oncogene FLEXIs were also potentially informative, as illustrated by scatter plots for the RBP-bindings sites that were enriched in oncogene FLEXIs that were 2-fold upregulated in MCF-7 or MDA-MB-231 cells or in all four cancer samples. These included proteins that function in or regulate transcription (GTF2F1), RNA splicing (KHSRP), RNA processing (CSTF2T), nuclear cap binding (NCBP2), cell cycle progression (TBRG4), and cell division (CDC40; FIG. 14E).

### DISCUSSION

Here, a new large class of human RNAs, short full-length excised linear intron RNAs at the transcriptome level were characterized. In total, including FLEXIs detected in both the initial cellular and plasma samples and the subsequent cancer samples, 8,687 different FLEXI RNAs expressed from 3,923 host genes representing-17% of the 51,645 short introns (300 nt) annotated in Ensembl GRCh38 Release 93 annotations were identified. Most FLEXI RNAs have relatively high GC content (60-70%) and are predicted to fold into stable RNA secondary structures (−20 to −50 kcal/mole). The detected FLEXI RNAs had cell- and tissue-specific expression patterns, reflecting differences in host gene transcription, alternative splicing, and intron RNA turnover, and they contained experimentally identified binding sites for diverse proteins, including transcription factors, chromatin remodeling proteins, and proteins that function in cellular stress responses, apoptosis, and cell proliferation, potentially linking FLEXI RNA binding to regulation of these processes. Their cell-specific expression patterns and origin from thousands of different protein-coding genes suggest that FLEXI RNAs may have utility as RNA biomarkers for human diseases.

Some of the FLEXI RNAs that were detected in human cells and plasma were known to have specialized biological functions as agotrons or as mirtron- or snoRNA-precursors and to bind specific non-splicing related RBPs needed to carry out these functions. Such binding could occur either before or after dissociation from the spliceosome, which may occur at different rates for different excised intron RNAs. To explore whether the much larger number of newly detected FLEXIs might include others that have other specialized biological functions, FLEXI RNA-binding proteins were searched in published CLIP-seq datasets. 126 different proteins that have annotated binding sites in FLEXI RNAs were identified, with 121 of these proteins binding multiple different FLEXI RNAs and 53 binding 30 or more different FLEXIs (FIG. 10 and FIG. 21). Based on the CLIP-seq datasets, spliceosomal proteins have annotated binding sites in the largest numbers of FLEXI RNAs, followed by AGO1-4 and DICER, with the latter including but not limited to annotated agotrons and mirtron pre-miRNAs (FIG. 10).

Surprisingly, 23 proteins that have CLIP-seq identified binding sites in 30 to 365 different FLEXIs have no known RNA splicing- or miRNA-related functions (Table 6), and 16 of these proteins were associated with distinct subsets of FLEXI RNAs that were under-represented in binding sites for spliceosomal proteins (FIG. 9D and FIG. 11). These RBPs included 16 that function in other processes unrelated to RNA splicing, including seven transcription regulators, four chromatin-binding or remodeling proteins, and two proteins that function in protein modification. Five of these proteins function in the regulation of apoptosis (AATF, BCLAFI, DDX3X, RPS3, ZNF622); four are regulators of p53 transcription or function (AATF, BCLAFI, DDX24, GRWD1); four function in DNA damage responses (AATF, BCLAF1, PABPN1, RPS3); four function in cellular stress responses (BCLAFI, G3BP1, SUB1, AATF); three function in cell growth regulation (AATF, DDX3X, IGF2BP1); and three play key roles in stress granule formation (DDX3X, TIA1, and G3BP1).

In general, FLEXI RNAs could contribute to cellular regulation by serving as substrates for DICER- or RNase III-cleavage to generate as yet unannotated small regulatory RNAs; by forming an RNP complex that functions in or regulates a process; by regulating of FLEXI host gene splicing, as found for alternative splicing factors that bind highly conserved FLEXI RNAs within protein-coding sequences (FIG. 9D and FIG. 12); by altering the subcellular localization of the bound protein, as found for a circular RNA linked to aberrant nuclear localization of DICER in glioblastoma (Bronisz et al. 2020); or by sequestering proteins, as suggested for the yeast linear intron RNAs that accumulate in stationary phase (Morgan et al. 2019; Parenteau et al. 2019).

Pertinent to their ability to function in cellular regulation, more than half of the newly identified FLEXIs were as abundant as mirtrons, agotrons, or biologically relevant snoRNAs in the same datasets (Martens-Uzunova et al. 2015; Hansen et al. 2016; Oliveira et al. 2021), with quantitative estimates based on sncRNAs with known copy number per cell values in the same datasets showing that the most abundant FLEXIs may be present at 1-2×10³ copies per cell and substantial numbers (20-87% in the different cellular RNA samples) may be present at 150 copies per cell (FIG. 7D and FIG. 20). Although most individual FLEXIs were not present in sufficiently high abundance to bind a substantial fraction of a typical intracellular target protein, collectively thousands of different FLEXIs have annotated binding sites for a small set of spliceosomal proteins and hundreds of different FLEXIs have annotated binding sites for DICER and AGO1-4. Thus in aggregate, FLEXI abundance could be sufficient to affect intracellular protein levels in response to stimuli that globally affect FLEXI RNA turnover, as found for the collective of yeast linear introns that accumulate under stress conditions (Morgan et al. 2019). The findings that key cellular regulatory proteins bind groups of 30 to 365 different FLEXIs (FIG. 10A) and that host genes encoding FLEXI RNAs bound by the same RBP have related and possibly coordinately regulated biological functions (FIG. 12) further show how the effects of FLEXI binding on the intracellular protein concentrations could be amplified to compensate for the relatively low abundance of some FLEXI RNAs.

With respect to evolution, although more conserved than other short introns, most of the detected FLEXI RNAs, including those corresponding to mirtron pre-miRNAs or agotrons, had relatively low PhastCons scores (<0.2; FIG. 7C, right panel), indicating either recent acquisition or rapid sequence divergence. FLEXI RNAs with relatively high PhastCons scores included those encoding snoRNAs, which contain binding sites for proteins involved in snoRNA biogenesis (FIG. 23), and those that are alternatively spliced to generate different protein isoforms, which contain binding sites for a distinct set of non-spliceosomal RBPs, including proteins known to function in alternative splicing (FIG. 9D).

In general, FLEXI RNAs can arise either by splice-site acquisition, as found for an EIFJ intron whose acquisition resulted in a novel human EIF1 isoform (Kim et al. 2020), or by an active intron transposition process, as found for spliceosomal introns in fungi, algae, and yeast (van der Burgt et al. 2012; Simmons et al. 2015; Lee and Stevens 2016). Most of the short introns in the human genome (97%) have unique sequences, with the remainder (1,719 introns with 693 unique sequences) arising by external or internal gene duplications, as described in detail for an abundant FLEXI RNA found in human plasma (Yao et al. 2020). Thus, if intron transposition is involved in the origin of FLEXIs, it must either be relatively rare or followed by rapid sequence divergence. The large number of FLEXIs encoded in the human genome may reflect that short introns within protein-coding sequences are more easily acquired or less deleterious for gene function than longer introns. Additional functions of FLEXI RNAs may arise secondarily and would be favored by stable predicted secondary structures that facilitate splicing by bringing splice sites closer together, contribute to the formation of protein-binding sites, and/or stabilize the intron RNA from turnover by cellular RNases, enabling them to persist long enough to perform their function after debranching.

Regardless of their function or origin, FLEXI RNAs constitute a large previously unidentified class of potential RNA biomarkers, with genome coverage comparable to mRNAs or miRNA. In addition to being linked to the transcription of thousands of protein-coding and lncRNA genes, FLEXI RNA levels can also reflect differences in alternative splicing and intron RNA stability (FIG. 8), providing higher resolution of cellular differences than mRNAs transcribed from the same gene. FLEXI RNAs may have particular utility as biomarkers in bodily fluids such as plasma, where they are enriched compared to other RNA species and their stable secondary structures and/or bound proteins may protect them from extracellular RNases (Yao et al. 2020). As many FLEXIs are predicted to fold into stable RNA secondary structures, their initial identification as candidate biomarkers seems best done by TGIRT-seq, which can yield full-length, end-to-end sequence reads of structured RNAs. Once identified, the validation of candidate FLEXI biomarkers and their routine monitoring in clinical samples can best be done by methods that give quantitative read outs for specific RNAs, such as RT-qPCR, microarrays, other hybridization-based assays, or targeted RNA-seq. Targeted RNA panels of FLEXI RNAs by themselves or together with other RNA biomarkers or analytes can provide a rapid cost-effective method for the diagnosis and routine monitoring of progression and response to treatment of a wide variety of human diseases.

### Materials and Methods for Example 2

### DNA and RNA Oligonucleotides

The DNA and RNA oligonucleotides used for TGIRT-seq on the Illumina sequencing platform are listed in Table 7. Oligonucleotides were purchased from Integrated DNA Technologies (IDT) in RNase-free, HPLC-purified form. R2R DNA oligonucleotides with 3′ A, C, G, and T residues were hand-mixed in equimolar amounts prior to annealing to the R2 RNA oligonucleotide.

RNA preparations 210. Universal Human Reference RNA (UHRR) was purchased from Agilent, and HeLa S3 and MCF-7 RNAs were purchased from Thermo Fisher. RNAs from matched frozen healthy/tumor tissues of breast cancer patients were purchased from Origene (500 ng; Patient A: PR+, ER+, HER2−, CR562524/CR543839; Patient B: PR unknown, ER−, HER2−, CR560540/CR532030).

211. K-562, HEK-293T/17, and MDA-MB-231 RNAs were isolated from cultured cells by using a mir Vana miRNA Isolation Kit (Thermo Fisher). K-562 cells (ATCC CTL-243) were maintained in Iscove's Modified Dulbecco's Medium (IMDM)+4 mM L-glutamine and 25 mM HEPES; Thermo Fisher) supplemented with 10% Fetal Bovine Serum (FBS; Gemini Bio-Products), and approximately 2×10⁶ cells were used for RNA extraction. HEK-293T/17 cells (ATCC CRL-11268) were maintained in Dulbecco's Modified Eagle Medium (DMEM)+4.5 g/L D-glucose, 4 mM L-glutamine, and 1 mM sodium pyruvate; Thermo Fisher) supplemented with 10% FBS, and approximately 4×10⁶ cells were used for RNA extraction. MDA-MB-231 cells (ATCC HTB-26) were maintained in DMEM+4.5 g/L D-glucose and 4 mM L-glutamine; Thermo Fisher) supplemented with 10% FBS and 1× PSQ (Penicillin, Streptomycin, and Glutamine: Thermo Fisher), and approximately 4×10⁶ cells were used for RNA extraction. All cells were maintained at 37° C. in a humidified 5% CO2 atmosphere.

For RNA isolation, cells were harvested by centrifugation (after trypsinization for HEK-293T/1 7 and MDA-MB-231 cells) at 300× g for 10 min at 4° C. and washed twice by centrifugation with cold Dulbecco's Phosphate Buffered Saline (Thermo Fisher). The indicated number of cells (see above) was then resuspended in 600 μL of mir Vana Lysis Buffer and RNA was isolated according to the kit manufacturer's protocol with elution in a final volume of 100 μL. To remove residual DNA, UHRR and HeLa S3 RNAs (1 μg) and patients A and B healthy and cancer tissue RNAs (500 ng) were treated with 20 U exonuclease I (Lucigen) and 2 U Baseline-ZERO DNase (Lucigen) in Baseline-ZERO DNase Buffer for 30 min at 37° C. K562, MDA-MB-231 and HEK-293T cell RNAs (5 μg) were incubated with 2 U TURBO DNase (Thermo Fisher). After DNA digestion, RNA was cleaned up with an RNA Clean & Concentrator kit (Zymo Research) with 8 volumes of ethanol (8× ethanol) added to maximize the recovery of small RNAs. The eluted RNAs were ribodepleted by using the rRNA removal section of a TruSeq Stranded Total RNA Library Prep Human/Mouse/Rat kit (Illumina), with the supernatant from the magnetic-bead separation cleaned-up by using a Zymo RNA Clean & Concentrator kit with 8× ethanol. After checking RNA concentration and length by using an Agilent 2100 Bioanalyzer with a 6000 RNA Pico chip, RNAs were aliquoted into −20 ng portions and stored at −80° C. until use.

For the preparation of samples containing chemically fragmented long RNAs, RNA preparations were treated with exonuclease I and Baseline-Zero DNase to remove residual DNA and ribodepleted, as described above. The supernatant from the magnetic-bead separation after ribodepletion was then cleaned-up with a Zymo RNA Clean & Concentrator kit using the manufacturer's two-fraction protocol, which separates RNAs into long and short RNA fractions (200-nt cut-off). The long RNAs were then fragmented to 70-100 nt by using an NEBNext Magnesium RNA Fragmentation Module (94° C. for 7 min; New England Biolabs). After clean-up by using a Zymo RNA Clean & Concentrator kit (8× ethanol protocol), the fragmented long RNAs were combined with the unfragmented short RNAs and treated with T4 polynucleotide kinase (Epicentre) to remove 3′ phosphates (Xu et al. 2019), followed by clean-up using a Zymo RNA Clean & Concentrator kit (8× ethanol protocol). After confirming the RNA fragment size range and RNA concentration by using an Agilent 2100 Bioanalyzer with a 6000 RNA Pico chip, the RNA was aliquoted into 4 ng portions for storage in −80° C.

### TGIRT-seq

TGIRT-seq libraries were prepared as described (Xu et al. 2019) using 20-50 ng of ribodepleted unfragmented RNA or 4-10 ng of ribodepleted chemically fragmented RNA The template-switching and reverse transcription reactions were done with 1 μM TGIRT-111 (InGex) and 100 nM pre-annealed R2 RNA/R2R DNA starter duplex in 20 μl of reaction medium containing 450 mM NaCl, 5 mM MgCh, 20 mM Tris-HCl, pH 7.5 and 5 mM DTT. Reactions were set up with all components except dNTPs, pre-incubated for 30 min at room temperature, a step that increases the efficiency of RNA-seq adapter addition by TGIRT template switching, and initiated by adding dNTPs (final concentrations 1 mM each of dATP, dCTP, dGTP, and dTTP). The reactions were incubated for 15 min at 60° C. and then terminated by adding 1 μl 5 M NaOH to degrade RNA and heating at 95° C. for 5 min followed by neutralization with 1 μl 5 M HCl and one round of MinElute column clean-up (Qiagen). The RIR DNA adapter was adenylated by using a 5′ DNA Adenylation kit (New England Biolabs) and then ligated to the 3′ end of the cDNA by using thermostable 5′ App DNA/RNA Ligase (New England Biolabs) for 2 hat 65° C. The ligated products were purified by using a MinElute Reaction Cleanup Kit and amplified by PCR with Phusion High-Fidelity DNA polymerase (Thermo Fisher Scientific): denaturation at 98° C. for 5 see followed by 12 cycles of 98° C. 5 see, 60° C. 10 see, 72° C. 15 see and then held at 4° C. The PCR products were cleaned up by using Agencourt AMPure XP beads (1.4× volume; Beckman Coulter) and sequenced on an Illumina NextSeq 500 to obtain 2×75 nt paired-end reads or on an Illumina NovaSeq 6000 to obtain 2×150 nt paired-end reads at the Genome Sequence and Analysis Facility of the University of Texas at Austin.

TGIRT-seq of RNA from commercial human plasma pooled from multiple healthy individuals was described previously (Yao et al. 2020), and the resulting datasets were previously deposited in the National Center for Biotechnology Information Sequence Read Archive under accession number PRJNA640428.

### Bioinformatics

All data analysis used combined TGIRT-seq datasets obtained from multiple replicates of different sample types (Table 4). Illumina TruSeq adapters and PCR primer sequences were trimmed from the reads with Cutadapt v2.8 (sequencing quality score cut-off at 20; p-value<0.01) (Martin 2011) and reads<15-nt after trimming were discarded. To minimize mismapping, a sequential mapping strategy was used. First, reads were mapped to the human mitochondrial genome (Ensembl GRCh38 Release 93) and the Escherichia coli genome (GeneBank: NC_000913) using HISAT2 v2.1.0 (Kim et al. 2019) with customized settings (−k 10—rfg 1,3—rdg 1,3—mp 4,2—no-mixed—no-discordant—no-spliced-alignment) to filter out reads derived from mitochondrial and E. coli RNAs (denoted Pass 1). Unmapped read from Pass1 were then mapped to a customized set of references sequences for genes encoding human sncRNAs (miRNA, tRNA, Y RNA, Vault RNA, 7SL RNA, 7SK RNA genes) and rRNAs (the 2.2-kb 5S rRNA repeats from the 5S rRNA cluster on chromosome 1 (lq42, GeneBank: X12811) and the 43-kb 45S rRNA containing 5.8S, 18S and 28S rRNAs from clusters on chromosomes 13,14, 15, 21, and 22 (GeneBank: U13369)), using HISAT2 with the following settings-k 20—rdg 1,3—rfg 1,3—mp 2, 1—no-mixed—no-discordant—no-spliced-alignment—norc (denoted Pass 2). Unmapped reads from Pass 2 were then mapped to the human genome reference sequence (Ensembl GRCh38 Release 93) using HISAT2 with settings optimized for non-spliced mapping (−k 10—rdg 1,3—rfg 1,3—mp 4,2—no-mixed—no-discordant—no-spliced-alignment) (denoted Pass 3) and splice aware mapping (−k 10—rdg 1,3—rfg 1,3—mp 4,2—no-mixed—no-discordant—dta) (denoted Pass 4). Finally, the remaining unmapped reads were mapped to Ensembl GRCh38 Release 93 by Bowtie 2 v2.2.5 (Langmead and Salzberg 2012) using local alignment (with settings as:—k 10—rdg 1,3—rfg 1,3—mp 4—ma 1—no-mixed—no-discordant—very-sensitive-local) to improve the mapping rate for reads containing post-transcriptionally added 5′ or 3′ nucleotides (poly(A) or poly(U)), short untrimmed adapter sequences, or non-templated nucleotides added to the 3′ end of the cDNAs by TGIRT-III during TGIRT-seq library preparation (denoted Pass 5). For reads that map to multiple genomic loci with the same mapping score in passes 3 to 5, the alignment with the shortest distance between the two paired ends (i.e., the shortest read span) was selected. In the case of ties (i.e., reads with the same mapping score and read span), reads mapping to a chromosome were selected over reads mapping to scaffold sequences, and in other cases, the read was assigned randomly to one of the tied choices. The filtered multiply mapped reads were then combined with the uniquely mapped reads from Passes 3-5 by using SAMtools v1.10 (Li et al. 2009) and intersected with gene annotations (Ensembl GRCh38 Release 93) with the RNY5 gene and its 10 pseudogenes, which are not annotated in this release, added manually to generate the counts for individual features. Coverage of each feature was calculated by BEDTools v2.29.2 (Quinlan 2014). To avoid miscounting reads with embedded sncRNAs that were not filtered out in Pass 2 (e.g., snoRNAs), reads were first intersected with sncRNA annotations and the remaining reads were then intersected with the annotations for protein-coding genes RNAs, lincRNAs, antisense RNAs, and other lncRNAs to get the read count for each annotated feature.

Coverage plots and read alignments were created by using Integrative Genomics Viewer v2.6.2 (IGV) (Robinson et al. 2011). Genes with>100 mapped reads were down sampled to 100 mapped reads in IGV for visualization.

To identify short introns that could give rise to FLEXI RNAs, intron annotations were extracted from Ensemble GRCh38 Release 93 gene annotation using a customized script and filtered to remove introns>300 nt as well as duplicate intron annotations from different mRNA isoforms. To identify FLEXI RNAs, mapped reads were intersected with the short intron annotations using BEDTools, and read pairs (Read 1 and Read 2) ending at or within 3 nucleotides of annotated 5′—and 3′-splice sites were identified as corresponding to FLEXI RNAs.

UpSet plots of FLEXI RNAs from different sample types were plotted by using the Complex Heatmap package v2.2.0 in R (Gu et al. 2016), and Venn diagrams were plotted by using the VennDiagram package v1.6.20 in R (Chen and Boutros 2011). For plots of FLEXI host genes, FLEXI RNAs were aggregated by Ensemble ID, and different FLEXI RNAs from the same gene were combined into one entry. Density distribution plots and scatter plots of log 2 transformed RPM of the detected FLEXI RNAs and FLEXI host genes were plotted by using R. PCA analysis of cell-type specific FLEXI RNA profiles in replicate cellular RNA datasets was plotted using R, and PCA initialized t-SNE and ZINB-WaVE analyses of these datasets were plotted using the Rtsne and zinbwave packages in R (Risso et al. 2018).

5′—and 3′-splice sites (SS) and branch-point (BP) consensus sequences of human U2—and U12-type spliceosomal introns were obtained from previous publications (Sheth et al. 2006; Gao et al. 2008). Splice-site consensus sequences of FLEXI RNAs were calculated from nucleotides frequencies of the first and last 10 nt from the intron ends. FLEXI RNAS corresponding to U12-type introns were identified by searching for (i) FLEXI RNAs with AU-AC ends and (ii) the 5′-splice site consensus sequence of U12-type introns with GU-AG ends (Sheth et al. 2006) using FIMO (Grant et al. 2011) with the following settings: FIMO—text—norc<GU_AG_U12_5 SS motif file><sequence file>. The branch-point consensus sequence of U2-type FLEXI RNAs was determined by searching for motifs enriched within 40 nt of the 3′ end of the introns using MEME (Bailey et al. 2009) with settings: meme<sequence file>-ma-oc<output folder>-mod anr-nmotifs 100-minw 6-minsites 100-markov order 1-evt 0.05. The branch-point consensus sequence of U12-type FLEXI RNAs (2 with AU-AC ends and 34 with GU-AG matching the 5′ sequence of GU-AG U12-type introns) was identified by manual sequence alignment and calculation of nucleotide frequencies. Motif logos were plotted from the nucleotide frequency tables of each motif using scripts from MEME suite (Bailey et al. 2009).

FLEXI RNAs corresponding to annotated mirtrons, agotrons, and RNA-binding-protein (RBP) binding sites were identified by intersecting the FLEXI RNA coordinates with the coordinates of annotated mirtrons (Wen et al. 2015), agotrons (Hansen et al. 2016), 150 RBPs (eCLIP, GENCODE, annotations with irreproducible discovery rate analysis) (Van Nostrand et al. 2016), DICER PAR-CLIP (Rybak-Wolf et al. 2014), and Ago1-4 PAR-CLIP (Hafner et al. 2010) datasets by using BEDTools. The functional annotations, localization patterns, and predicted RNA-binding domains of the 150 RBPs in the ENCODE eCLIP dataset were based on Table 5 of (Van Nostrand et al. 2020). RBPs found in stress granules were as annotated in the RNA Granule and Mammalian Stress Granules Proteome (MSGP) databases (Nunes et al. 2019; Youn et al. 2019). The functional annotations, localization patterns, and RNA-binding domains of AGO1-4 and DICER were retrieved from the UniProt database (The UniProt Consortium 2018). FLEXI RNAs containing embedded snoRNAs were identified by intersecting the FLEXI RNA coordinates with the coordinates of annotated snoRNA and scaRNA from Ensembl GRCh38 annotations.

GO enrichment analysis of host genes encoding FLEXIs bound by different RBPs was performed using DAVID bioinfonnatics tools (Huang et al. 2009) with all FLEXI host genes as the background. Hierarchical clustering was performed based on p-values for GO tem1 enrichment of FLEXI host genes bound by the same RBPs using the Seaborn ClusterMap package in Python.

### Data Access

TGIRT-seq datasets have been deposited in the Sequence Read Archive (SRA) under accession numbers PRJNA648481 and PRJNA.640428. A gene counts table, dataset metadata file, FLEXI metadata file, RBP annotation file, and scripts used for data processing and plotting have been deposited in GitHub.

TABLE 4
Summary of datasets for example 2.

| | Raw reads | Trimmed | Mapped | Mapped to |
|---|---|---|---|---|
| RNA origin | (×10⁶} | reads × 10⁶} | reads × 10⁶} | feature × 10⁶} |
| HEK-293T* | 741.6 | 726.5 | 715.2 | 690.9 |
| | | (98.0%) | (98.5%) | (96.6%) |
| HeLa s3t | 851.0 | 803.3 | 768.4 | 705.6 |
| | | (94.4%) | (95.7%) | (92.0%) |
| UHRRt | 712.0 | 682.2 | 666.3 | 630.9 |
| | | (95.8%) | (97.7%) | (94.7%) |
| K-562§ | 744.2 | 725.0 | 713.8 | 698.5 |
| | | (97.4%) | (98.4%) | (97.9%) |
| Plasmaif | 232.5 | 122.7 | 71.1 | 61.7 |
| | | (91.3%) | (57.9%) | (87.2%) |
| MDA-MB-231# | 226.1 | 211.3 | 207.5 | 202.2 |
| | | (93.5%) | (98.2%) | (97.5%) |
| MCF711 | 757.8 | 703.7 | 692.1 | 673.7 |
| | | (92.9%) | (98.4%) | (97.3%) |
| Patient A Healthy** | 338.3 | 312.6 | 305.1 | 297.6 |
| | | (92.4%) | (97.6%) | (97.5%) |
| Patient A Cancer** | 295.8 | 275.0 | 268.2 | 256.5 |
| | | (93.0%) | (97.5%) | (95.6%) |
| Patient B Healthy** | 281.9 | 258.2 | 251.6 | 244.0 |
| | | (91.6%) | (97.4%) | (97.0%) |
| Patient B Cancer** | 549.7 | 492.4 | 477.5 | 461.6 |
| | | (89.6%) | (97.0%) | (96.7%) |
| Patient A Healthy (Fragmented) tt | 55.7 | 52.7 | 50.5 | 33.2 |
| | | (94.7%) | (95.8%) | (60.1%) |
| Patient A Cancer (Fragmented) tt | 61.9 | 59.8 | 57.0 | 40.5 |
| | | (96.7%) | (95.3%) | (68.8%) |
| Patient B Healthy (Fragmented) tt | 39.6 | 35.1 | 33.1 | 22.0 |
| | | (88.5%) | (94.3%) | (57.2%) |
| Patient B Cancer (Fragmented) tt | 58.4 | 56.9 | 54.1 | 47.1 |
| | | (97.5%) | (95.1%) | (85.5%) |

*HEK-293T cell RNA, combined datasets from 8 replicates (HEK-repl to 8).t HeLa S3 cell RNA, combined datasets from 10 replicates (HeLa-repl to 10).
tUniversal human reference RNA, combined datasets from 8 replicates (UHRR-repl to 8).
§K-562 cell RNA, combined datasets from 8 replicates (K-562-repl to 8).
_{i, i} Commercial human plasma pooled plasma from healthy individuals. Fifteen combined datasets (SRA BioProject accession number PRJNA640428, samples DNasel_1-12, Exol_1-3) (Yao et al. 2020).
#MDA-MB-231 RNA, combined datasets from 2 replicates (MDA-repl and 2).
II MCF7 RNA, combined datasets from 8 replicates (MCF-repl to 8).
**Matched healthy and cancer breast tissue RNA from patients A and B purchased from Origene (Patient A: PR+, ER+, HER2−; CR562524/CR543839); Patient B: PR unknown, ER−, HER2−; CR560540/CR532030). Combined datasets from 3 replicates for each sample type (rep1-3).
¹tt Chemically fragmented RNAs from matched healthy/cancer tissues from patients A and B.

TABLE 5
Abundance of sncRNAs (RPM) detected by TGIRT-seq in cellular RNA samples
compared to reported copy number per cell values for these RNAs.

| | RPM | | | | | |
|---|---|---|---|---|---|---|
| | | HEK- | HeLa | | | |
| Name | K-562 | 293T | S3 | UHRR | Copies/cell* | Type |
| U1 | 1,386 | 1,267 | 1,337 | 2,649 | 1 × 10⁶ | Major spliceosomal snRNA |
| U2 | 5,768 | 9,482 | 28,781 | 9,794 | 5 × 10⁵ | Major spliceosomal snRNA |
| U4 | 65 | 34 | 540 | 76 | 2 × 10⁵ | Major spliceosomal snRNA |
| U5 | 1,147 | 2,704 | 13,244 | 2,717 | 2 × 10⁵ | Major spliceosomal snRNA |
| | | | | | | Minor spliceosomal snRNA |
| U6 | 974 | 1,767 | 923 | 1,356 | 4 × 10⁵ | Major spliceosomal snRNA |
| U7 | 3 | 4 | 49 | 3 | 4 × 10³ | U7 |
| U11 | 2 | 3 | 11 | 8 | 1 × 10⁴ | Minor spliceosomal snRNA |
| U12 | 36 | 24 | 192 | 41 | 5 × 10³ | Minor spliceosomal snRNA |
| U4ATAC | 37 | 21 | 1,292 | 32 | 2 × 10³ | Minor spliceosomal snRNA |
| U6ATAC | 204 | 286 | 700 | 268 | 2 × 10³ | Minor spliceosomal snRNA |
| RN7SK | 1,369 | 2,773 | 5,044 | 7,180 | 2 × 10⁵ | 7SK |
| RN7SL | 3,447 | 8,085 | 4,690 | 19,815 | 5 × 10⁵ | 7SL |
| RPPH1 | 165 | 245 | 257 | 1,294 | 2 × 10⁵ | RNase P RNA component |
| SNORD3 | 7,637 | 12,699 | 23,195 | 38,533 | 2 × 10⁵ | CID box snoRNA, U3 |
| SNORD118 | 212 | 976 | 546 | 741 | 4 × 10⁴ | CID box snoRNA, US |
| SNORD13 | 435 | 366 | 817 | 1,221 | 4 × 10⁴ | CID box snoRNA, U13 |
| SNORD14 | 4,281 | 3,890 | 9,982 | 11,081 | 1 × 10⁴ | CID box snoRNA, U14 |
| SNORD22 | 362 | 241 | 748 | 948 | 1 × 10⁴ | CID box snoRNA, U22 |
| RMRP | 670 | 1,777 | 2,211 | 3,279 | 1 × 10⁵ | MRP |

*Copy number per cell values for sncRNAs in vertebrate cells (Tycowski et al. 2006) that were used in the linear regression analysis of FIG. 20.

TABLE 6
Proteins with no known RNA splicing- or miRNA-related
function that bind 30 or more different FLEXIs.

| Symbol | Name | Function |
|---|---|---|
| AATF | Apopwsis Antagonizing Transcription | Transcriptional cofactor with roles in |
| | Factor | cell proliferation, apoptosis, DNA |
| | | damage response and general stress |
| | | response through regulation of Rb, |
| | | HDACl, and p53 functions. |
| BCLAFl | BCL2-2-associated transcription factor 1 | Transcriptional repressor; promotes |
| | | apoptosis through interaction with |
| | | BCL2; Upregulated in senescence, |
| | | promotes p53 transcription in response |
| | | to DNA damage. |
| DDX24 | ATP-dependent RNA helicase DDX24 | ATP-dependent RNA helicase and |
| | | negative regulator of p53 |
| DDX3X | ATP-dependent RNA helicase DDX3X | Multifunctional ATP-dependent RNA |
| | | helicase with functions in cell cycle |
| | | control, apoptosis, and innate |
| | | immunity; critical role in stress |
| | | granule assembly. |
| DDX55 | ATP-dependent RNA helicase DDX55 | Probable ATP-binding RNA helicase |
| DKCl | H/ACA ribonucleoprotein complex | Catalytic subunit of H/ACA small |
| | subunit DKCl | nucleolar ribonucleoprotein (H/ACA |
| | | snoRNP) complex; plays an active role |
| | | in telomerase stabilization |
| FXR2 | Fragile X mental retardation syndrome- | RNA-binding protein |
| | related protein 2 | |
| G3BP1 | Ras GTPase-activating protein-binding | ATP- and Mg-dependent helicase that |
| | protein 1 | plays an essential role in innate |
| | | immunity; Also functions in stress |
| | | granule assembly and is associated with |
| | | cellular senescence. Regulates Ras, |
| | | TGF-P/Smad, Src/FAK and p53 |
| | | signaling pathways. |
| GRWDl | Glutamate-rich WD repeat-containing | Hi stone binding-protein that regulates |
| | protein 1 | chromatin dynamics and |
| | | minichromosome maintenance (MCM) |
| | | loading at replication origins; |
| | | negatively regulates p53. |
| IGF2BP1 | Insulin-like growth factor 2 mRNA- | RNA-binding protein that recruits |
| | binding protein 1 | target transcripts to cytoplasmic |
| | | protein-RNA complexes (mRNPs); |
| | | Promotes cell cycle progression |
| | | through regulation of E2F translation. |
| LARP4 | La-related protein 4 | RNA binding protein that binds to the |
| | | polyA tract of mRi′\fA molecules |
| LSMll | U7 snRNA-associated Sm-like protein | Component of the 1)7 snRNP complex |
| | LSmll | that is involved in the histone 3′-end |
| | | pre-rnRNA processing |
| METAP2 | Methionine aminopeptidase 2 | Co-translationally removes the N- |
| | | terminal methionine from nascent |
| | | protems. |
| NOLCl | Nucleolar and coiled-body | Nucleolar protein that plays a critical |
| | phosphoprotein 1 | role in snoRNP assembly and acts as a |
| | | regulator of RNA polymerase I by |
| | | connecting RNA polymerase I with |
| | | enzymes responsible for ribosomal |
| | | processing and modification; Stabilizes |
| | | telomeres by regulating TRF2 |
| | | retention. |
| PABPC4 | Polyadenylate-binding protein 4 | Binds the poly A tail of mRNA |
| PABPNl | Polyadenylate-binding protein 2 | Involved in the 3′-end formation of |
| | | mRNA precursors (pre-mRNA) by the |
| | | addition of a poly(A) tail; Regulated by |
| | | ATM and plays a crucial role in DSB |
| | | repa.ir. |
| RPS3 | 40S ribosomal protein S3 | Role in regulating transcription; |
| | | implicated in regulating DNA damage |
| | | response and apoptosis. |
| SUBl | Activated RNA polymerase II | General coactivator that functions |
| | transcriptional coactivator pl5 | cooperatively with TAFs and mediates |
| | | functional interactions between |
| | | upstream activators and the general |
| | | transcriptional machinery; critical role |
| | | in genome integrity and chromatin |
| | | compaction, regulates transcription in |
| | | response to stress. |
| UCHL5 | Ubiquitin carboxyl-terminal hydrolase | Protease |
| | isozyme LS | |
| XRN2 | 5′-3′ exoribonuclease 2 | May promote the termination of |
| | | transcription by Rl″-lA polymerase II |
| YBX3 | Y-Box-binding protein 3 | Binds also to full-length mRNA and to |
| | | short RNA sequences containing the |
| | | consensus site 5′-UCCAUCA-3′ (SEQ |
| | | ID NO: 8) |
| ZNF622 | Zinc finger protein 622 | May behave as an activator of the |
| | | bound transcription factor, MYBL2; |
| | | positive regulator of apoptosis. |
| ZNF800 | Zinc finger protein 800 | May be involved in transcriptional |
| | | regulation. |

TABLE 7
Oligonucleotides used in example 2 for construction of TGIRT-seq libraries

| Name | Sequence and notes |
|---|---|
| NTTR2RNA | 5′-AAGAUCGGAAGAGCACACGUCUGAACUCCAGUCAC/3SpC/ |
| | (SEQ ID NO: 9) |
| NTTR2RDNA | 5′-GTGACTGGAGTTCAGACGTGTGCTCTTCCGATCTTN-3′, where N |
| | is an equimolar mix of A, C, G, T (obtained by hand mixing of individual |
| | oligonucleotides with A, C, G and T at their 3′ end). (SEQ ID NO: 10) |
| RlRDNA | RlRDNA: 5′-/5Phos/GATCGTCGGACTGTAGAACTCTGAACGTGT |
| | AG/3SpC3/. (SEQ ID NO: 11) The RlR oligonucleotide was adenylated. as |
| | described in Materials and Methods. |
| Illumina | 5′-AATGATACGGCGACCACCGAGATCTACACGTTCAGAGTTCTA |
| multiplex PCR | CAGTCCGACGATC-3′ (SEQ ID NO: 12) |
| pnmer | |
| Illumina index | 5′CAAGCAGAAGACGGCATACGAGATBARCODE*GTGACTGGA |
| PCRprimer | GTTCAGACGTGTGCTCTTCCGATCT-3′ (SEQ ID NO: 13), where |
| | BARCODE* corresponds to the 6 nucleotide Illumina TruSeq barcode |
| | sequence. |

### G. REFERENCES

Berezikov, E., Chung, W.-J., Willis, J., Cuppen, E., and Lai, E. C. (2007). Mammalian Mirtron Genes. Molecular Cell 28, 328-336.
Blocker, F. J. H., Mohr, G., Conlan, L. H., Qi, L., Belfort, M., and Lambowitz, AM. (2005) Domain structure and three-dimensional model of a group II intron-encoded reverse transcriptase. RNA 11, 14-28.
Chapman, K. B., and Boeke, J. D. (1991). Isolation and characterization of the gene encoding yeast debranching enzyme. Cell 65, 483-492.
Burset, M., Seledtsov, I. A, and Solovyev, V. V. (2000). Analysis of canonical and non-canonical splice sites in mammalian genomes. Nucleic Acids Research 28, 4364-4375.
Chorev M, Carmel L. The function of introns. Front Genet. 2012; 3:55. Published 2012 Apr 13.
Hansen, T. B. (2018). Detecting Agotrons in Ago CLIPseq Data. Methods in Molecular Biology 1823, 221-232.
Gardner, E. J., Nizami, Z. F., Talbot Jr., C. C., and Gall, J. G. (2012). Stable intronic sequence RNA (sisRNA), a new class of noncoding RNA from the oocyte nucleus of Xenopus tropicalis. Genes & Dev. 26, 2550-2559.
Hansen, T. B., Ven0, M. T., Jensen, T. I., Schaefer, A, Damgaard, C. K., and Kjems, J. (2016). Argonaute-associated short introns are a novel class of gene regulators. Nature Communications 7, 11538.
Katibah, G. E., Qin, Y., Sidote, D. J., Yao, J., Lambowitz, A M., and Collins, K. (2014). Broad and adaptable RNA structure recognition by the human interferon-induced tetratricopeptide repeat protein IFIT5. Proc. Natl. Acad. Sci., USA 111, 12025-12030.
Kim, D., Paggi, J. M., Park, C., Bennett, C., and Salzberg, S.L. (2019). Graph-based genome alignment and genotyping with HISAT2 and HISAT-genotype. Nature Biotechnology 37,907-915.
Lambowitz, A M., and Zimmerly, S. (2011). Cold Spring Harb. Perspect. Biol. 2011; 3:a003616.
Langmead, B., and Salzberg, S. L. (2012). Fast gapped-read alignment with Bowtie 2. Nature Methods 9, 357-359.
Lentzsch, A M., Yao, J., Russell, R., and Lambowitz, AM. (2019). Template switching mechanism of a group II intron-encoded reverse transcriptase and its implications for biological function and RNA-seq. J. Biol. Chem. 294, 19764-19784.
Li, H., Handsaker, B., Wysoker, A, Fennell, T., Ruan, J., Homer, N., Marth, G., Abecasis, G., and Durbin, R. (2009). The sequence alignment/map format and SAMtools. Bioinformatics 25, 2078-2079.
Martin, M. (2011). Cutadapt removes adapter sequences from high-throughput sequencing reads. EMBnetjournal 17, pp. 10-12.
Mohr, S., Ghanem, E., Smith, Y., Sheeter, D., Qin, Y., King, O., Polioudakis, D., Iyer, V., Hunicke-Smith, S., Swamy, S., Kuersten, S., and Lambowitz, AM. (2013). RNA 19, 958-970.
Nottingham, RM., Wu, D. C., Qin, Y., Yao, J., Hunicke-Smith, S., and Lambowitz, A M. (2016). RNA-seq of human reference RNA samples using a thermostable group II intron reverse transcriptase. RNA 22, 597-613.
Morgan, J. T., Fink, G. R, and Bartel, D.P. (2019). Excised linear introns regulate growth in yeast. Nature 565, 606-611.
Okamura, K., Hagen, J. W., Duan, H., Tyler, D. M., and Lai, E. C. (2007). The mirtron pathway generates microRNA-class regulatory RNAs in Drosophila.Cell 130, 89-100.
Parenteau, J., Maignon, L., Berthoumieux, M., Catala, M., Gagnon, V., and Abou Elela, S. (2019). Introns as mediators of cell response to starvaton. Nature 565, 612-617.
Pek, J. W., Osman, I., Tay, M. L., and Zheng, R T. (2015). Stable intronic sequence RNAs have possible regulatory roles in Drosophila melanogaster. J. Cell Biol. 211, 243-251.
Qin, Y., Yao, J., Wu, D. C., Nottingham, R M., Mohr, S., Hunicke-Smith, S., and Lambowitz, A M. (2016). High-throughput sequencing of human plasma RNA by using thermostable group II intron reverse transcriptases. RNA 22, 111-128.
Quinlan, A R (2014). BEDTools: the swiss-army tool for genome feature analysis. Current Protocols in Bioinformatics 47, 11.12.11-34.
Rearick et al. Critical Association of ncRNA with Introns; Nucleic Acids Res. 39, 2357-2366 2011
Ruby, J. R, Jan, C. H., and Bartel, D.P. (2007). Intronic microRNA precursors that bypass Drosha processing. Nature 448, 83-86.
Saini, H., Bicknell, A. A, Eddy, S. R, and Moore, M. J. (2019). Free circular introns with an unusual branchpoint in neuronal projections. eLife 2019;8:e47809.
Shurtleff, M. J., Yao, J., Qin, Y., Nottingham, RM., Temoche-Diaz, M.M., Schekman, R, and Lambowitz, AM. (2017). Broad role for YBX1 in defining the small noncoding RNA composition of exosomes. Proceedings of the National Academy of Sciences 114, E8987-E8995.
Stamos, J. L., Lentzsch, A M., and Lambowitz, AM. (2017). Structure of a thermostable group II intron reverse transcriptase with template-primer and its functional and evolutionary implications. Molecular Cell 68, 926-939.
Talhouame, G. J. S., and Gall, J. G. (2018). Lariat intronic RNAs in the cytoplasma of vertebrate cells. Proc. Natl. Acad. Sci., U.S.A. 115, E7970-7977.
Wen, J., Ladewig, E., Shenker, S., Mohammed, J., and Lai, E. C. (2015). Analysis of Nearly One Thousand Mammalian Mirtrons Reveals Novel Features of Dicer Substrates. PLOS Computational Biology 11, el004441.
Wilkinson, M. E., Charenton, C., and Nagai, K. (2020). RNA splicing by the spliceosome. Annu. Rev. Biochem. 89, 1.1-1.30.
Xu, H., Yao, J., Wu, D. C., and Lambowitz, AM. (2019). Improved TGIRT-seq methods for comprehensive transcriptome profiling with decreased adapter dimer formation and bias correction. Scientific Reports 9, 7953.
Yu, G., Wang, L.-G., Han, Y., and He, Q.-Y. (2012). clusterProfiler: an R Package for Comparing Biological Themes Among Gene Clusters. OMICS: A Journal of Integrative Biology 16, 284-287.
Zhang, Y., Zhang, X.-Q., Chen, T., Xiang, J.-F., Yin, Q.-F., Xing, Y.-H., Zhu, S., Yang, L., and hen, L.-L. Circular intronic long noncoding RNAs. Molecular Cell 51, 792-806, 2013.
Zuker, M., and Stiegler, P. (1981). Optimal computer folding of large RNA sequences using thermodynamics and auxiliary information. Nucleic Acids Research 9, 133-148.
Bailey TL, Boden M, Buske F A, Frith M, Grant CE, Clementi L, Ren J, Li WW, Noble W S. 2009. MEME Suite: tools for motif discovery and searching. Nucleic Acids Res 37: W202-W208.
Bronisz A, Rooj A K, Krawczynski K, Peruzzi P, Salin.ska E, Nakano I, Purow B, Chiocca E A, Godlewski J. 2020. The nuclear DICER-circular RNA complex drives the deregulation of the glioblastoma cell microRNAome. Sci Adv 6: eabc0221.
Chen H, Boutros P C. 2011. VennDiagram: a package for the generation of highly-customizable Venn and Euler diagrams in R. BMC Bioinformatics 12: 35.
Biermann N, Haneke K, Sun Z, Stoecklin G, Ruggieri A 2020. Dance with the devil: stress granules and signaling in antiviral responses. Viruses 12.
Farrell M J, Dobson A T, Feldman L T. 1991. Herpes simplex virus latency-associated transcript is a stable intron. Proc Natl Acad Sci USA 88: 790-794.
Gao K, Masuda A, Matsuura T, Ohno K. 2008. Human branch point consensus sequence is yUnAy. Nucleic Acids Res 36: 2257-2267.
Gao X, Hardwidge PR 2011. Ribosomal protein s3: a multifunctional target of attaching/effacing bacterial pathogens. Front Microbiol 2: 137-137.
Gavish-Izakson M, Velpula B B, Elkon R, Prados-Carvajal R, Barnabas G D, Ugalde A P, Agami R, Geiger T, Huertas P, Ziv Yet al. 2018. Nuclear poly(A)—binding protein 1 is an ATM target and essential for DNA double-strand break repair. Nucleic Acids Res 46: 730-747.
Grant C E, Bailey TL, Noble W S. 2011. FIMO: scanning for occurrences of a given motif. Bioinformatics 27: 1017-1018.
Gu Z, Eils R, Schlesner M. 2016. Complex heatmaps reveal patterns and correlations in multidimensional genomic data. Bioinformatics 32: 2847-2849.
Hafner M, Landthaler M, Burger L, Khorshid M, Hausser J, Berninger P, Rothballer A, Ascano M, Jr., Jungkamp A-C, Munschauer Met al. 2010. Transcriptome-wide identification of RNA-binding protein and microRNA target sites by PAR-CLIP. Cell 141: 129-141.
Hilliker A, Gao Z, Jankowsky E, Parker R. 2011. The DEAD-box protein Dedl modulates translation by the formation and resolution of an eIF4F-mRNA complex. Mol Cell 43: 962-972.
Huang D W, Sherman B T, Lempicki R A 2009. Systematic and integrative analysis of large gene lists using DAVID bioinformatics resources. Nat Protoc 4: 44-57.
Iezzi S, Fanciulli M. 2015. Discovering Che-1/AATF: a new attractive target for cancer therapy. Front Genet 6.
Kaiser R W J, Ignarski M, Van Nostrand E L, Frese C K, Jain M, Cukoski S, Heinen H, Schaechter M, Seufert L, Bunte Ket al. 2019. A protein-RNA interaction atlas of the ribosome biogenesis factor AATF. Sci Rep 9: 11071.
Kedersha N L, Gupta M, Li W, Miller I, Anderson P. 1999. RNA-binding proteins TIA-1 and TIAR link the phosphorylation of eIF-2 alpha to the assembly of mammalian stress granules. J Cell Biol 147: 1431-1442.
Kim P, Yang M, Yiya K, Zhao W, Zhou X. 2020. ExonSkipDB: functional annotation of exon skipping event in human. Nucleic Acids Res 48: D896-D907.
Kobak D, Berens P. 2019. The art of using t-SNE for single-cell transcriptomics. Nat Commun 10: 5416.
Kufel J, Grzechnik P. 2019. Small nucleolar RNAs tell a different tale. Trends Genet 35: 104-117.
Kulesza C A, Shenk T. 2006. Murine cytomegalovirus encodes a stable intron that facilitates persistent replication in the mouse. Proc Natl Acad Sci USA 10^{3 : 18302}-18307.
Langmead B, Salzberg S L. 2012. Fast gapped-read alignment with Bowtie 2. Nat Methods 9: 357-359.
Lee S, Stevens S W. 2016. Spliceosomal introno genesis. Proc Natl Acad Sci USA 113: 6514-6519.
Liberzon A, Birger C, Thorvaldsdottir H, Ghandi M, Mesirov J P, Tamayo P. 2015. The Molecular Signatures Database (MSigDB) hallmark gene set collection. Cell Syst 1: 417-425.
Liu Y, Sun J, Zhao M. 2017. ONGene: A literature-based database for human oncogenes. J Genet Genomics 44: 119-121.
MacNeil D E, Lambert-Lanteigne P, Autexier C. 2019. N-terminal residues of human dyskerin are required for interactions with telomerase RNA that prevent RNA degradation. Nucleic Acids Res 47: 5368-5380.
Martens-Uzunova E S, Hoogstrate Y, Kalsbeek A, Pigmans B, Vredenbregt-van den Berg M, Dits N, Nielsen S J, Baker A, Visakorpi T, Bangma C et al. 2015. CID-box snoRNA-derived RNA production is associated with malignant transformation and metastatic progression in prostate cancer. Oncotarget 6: 17430-17444.
Morgan J T, Fink G R, Bartel D P. 2019. Excised linear introns regulate growth in yeast. Nature 565: 606-611.
Moss W N, Steitz J A. 2013. Genome-wide analyses of Epstein-Barr virus reveal conserved RNA structures and a novel stable intronic sequence RNA BMC Genomics 14: 543.
Muller S, Bley N, Busch B, Gla. 13 M, Lederer M, Misiak C, Fuchs T, Wedler A, Haase J, Bertoldo J B et al. 2020. The oncofetal RNA-binding protein IGF2BP1 is a druggable, post-transcriptional super-enhancer of E2F-driven gene expression in cancer. Nucleic Acids Res 48: 8576-8590.
Nunes C, Mestre I, Marcelo A, Koppenol R, Matos C A, Nobrega C. 2019. MSGP: the first database of the protein components of the mammalian stress granules. Database 2019.
Oliveira D, Prahm K P, Christensen I J, Hansen A, HOgdall C K, HOgdall E V. 2021. Noncoding RNA (ncRNA) profile association with patient outcome in epithelial ovarian cancer cases. Reprod Sci 28: 757-765.
Omer A, Barrera M C, Moran J L, Lian X J, Di Marco S, Beausejour C, Gallouzi I E. 2020. G3BP1 controls the senescence-associated secretome and its impact on cancer progression. Nat Commun 11: 4979.
Risso D, Perraudeau F, Gribkova S, Dudoit S, Vert J-P. 2018. A general and flexible method for signal extraction from single-cell RNA-seq data. Nat Commun 9: 284.
Robinson J T, Thorvaldsdottir H, Winckler W, Guttman M, Lander E S, Getz G, Mesirov J P. 2011. Integrative genomics viewer. Nat Biotechnol 29: 24-26.
Rybak-Wolf A, Jens M, Murakawa Y, Herzog M, Landthaler M, Rajewsky N. 2014. A variety of Dicer substrates in human and C.elegans. Cell 159: 1153-1167.
Schroder M. 2010. Human DEAD-box protein 3 has multiple functions in gene regulation and cell cycle control and is a prime target for viral manipulation. Biochem Pharmacol 79: 297-306.
Sheth N, Roca X, Hastings M L, Roeder T, Krainer A R, Sachidanandam R. 2006. Comprehensive splice-site analysis using comparative genomics. Nucleic Acids Res 34: 3955-3967.
Simmons M P, Bachy C, Sudek S, van Baren M J, Sudek L, Ares M, Jr, Worden A′Z. 2015. Intron invasions trace algal speciation and reveal nearly identical arctic and antarctic micromonas populations. Mol Biol Evol 32: 2219-2235.
Somasekharan S P, El-Naggar A, Leprivier G, Cheng H, Hajee S, Grunewald T G P, Zhang F, Ng T, Delattre O, Evdokimova Vet al. 2015. YB-1 regulates stress granule formation and tumor progression by translationally activating G3BP1. J Cell Biol 208: 913-929.
Sugimoto N, Maehara K, Yoshida K, Yasukouchi S, Osano S, Watanabe S, Aizawa M, Yugawa T, Kiyono T, Kurumizaka H et al. 2015. Cdtl-binding protein GRWD1 is a novel histone-binding protein that facilitates MCM loading through its influence on chromatin architecture. Nucleic Acids Res 43: 5898-5911.
The UniProt Consortium. 2018. UniProt: a worldwide hub of protein knowledge. Nucleic Acids Res 47: D506-D515.
Tycowski K T, Kolev N G, Conrad N K, Fok V, Steitz J A. 2006. The ever-growing world of small nuclear ribonucleoproteins. In The RNA World, Third Edition, (ed. RF Gesteland, et al.), pp. 327-368. Cold Spring Harbor Laboratory Press, NY.
van der Burgt A, Severing E, de Wit Pierre J G M, Collemare J. 2012. Birth of new spliceosomal introns in fungi by multiplication of introner-like elements. Curr Biol 22: 1260-1265.
Van Nostrand E L, Pratt G A, Shishkin A A, Gelboin-Burkhart C, Fang MY, Sundararaman B, Blue S M, Nguyen T B, Surka C, Elkins Ket al. 2016. Robust transcriptome-wide discovery of RNA-binding protein binding sites with enhanced CLIP (eCLIP). Nat Methods 13: 508-514.
Van Nostrand E L, Pratt G A, Yee B A, Wheeler E C, Blue S M, Mueller J, Park S S, Garcia K E, Gelboin-Burkhart C, Nguyen TB et al. 2020. Principles of RNA processing from analysis of enhanced CLIP maps for 150 RNA binding proteins. Genome Biol 21: 90.
Vohhodina J, Barros E M, Savage A L, Liberante F G, Manti L, Bankhead P, Cosgrove N, Madden A F, Harkin D P, Savage K I. 2017. The RNA processing factors THRAP3 and BCLAFI promote the DNA damage response through selective mRNA splicing and nuclear export. Nucleic Acids Res 45: 12816-12833.
Yao J, Wu D C, Nottingham R M, Lambowitz A M. 2020. Identification of protein-protected mRNA fragments and structured excised intron RNAs in human plasma by TGIRT-seq peak calling. eLife 9: e60743.
Youn J-Y, Dyakov B J A, Zhang J, Knight J D R, Vernon R M, Forman-Kay J D, Gingras A-C. 2019. Properties of stress granule and P-body proteomes. Mol Cell 76: 286-294.
Yuan F, Li G, Tong T. 2017. Nucleolar and coiled-body phosphoprotein 1 (NOLC1) regulates the nucleolar retention of TRF2. Cell Death Discov 3: 17043.
Zhang C-H, Wang J-X, Cai M-L, Shao R, Liu H, Zhao W-L. 2019. The roles and mechanisms of G3BP1 in tumour promotion. J Drug Target 27: 300-305.
Zhao M, Kim P, Mitra R, Zhao J, Zhao Z. 2016. TSGene 2.0: an updated literature-based knowledgebase for tumor suppressor genes. Nucleic Acids Res 44: D1023-D1031.

1. A method of determining one or more biomarkers in Full-Length Excised Linear Intron RNAs (FLEXI RNAs), wherein said one or more biomarkers are indicative of a specific characteristic, trait, disease, disorder or condition, the method comprising:
a obtaining FLEXI RNAs from one or more subjects with a specific characteristic, trait, disease, disorder or condition;
b. determining the sequence or sequences of the FLEXI RNAs from said one or more subjects;
c. comparing the sequence or sequences of said FLEXI RNAs from subjects with a specific characteristic, trait, disease, disorder or condition to sequences of control FLEXI RNAs to determine differences; and
d. determining which differences are indicative of a specific characteristic, trait, disease, disorder or condition, thereby identifying biomarkers for said specific characteristic, trait, disease, disorder or condition.

2. The method of claim 1, wherein said FLEXI RNAs are sequenced by RNA sequencing.

3. The method of claim 1, wherein said FLEXI RNAs are sequenced by using a non-LTR-retroelement reverse transcriptase-based method.

4. The method of claim 3, wherein the non-LTR retroelement reverse transcriptase is a group II intron-encoded reverse transcriptase.

5. The method of claim 1, wherein said specific disease is cancer, an infectious disease, an autoimmune disease, tissue damage, or mental disease.

6. (canceled)

7. The method of claim 1, wherein said biomarker is a predictive, diagnostic, or prognostic biomarker.

8. (canceled)

9. (canceled)

10. The method of claim 1, wherein said biomarker relates to a drug interaction, drug response, or heritable condition.

11. (canceled)

12. (canceled)

13. The method of claim 1, wherein said biomarker is used to track disease progression and/or response to treatment in a subject.

14. The method of claim 1, comprising determining two or more FLEXI RNA biomarkers.

15. The method of claim 14, wherein when at least two biomarkers are present together, they are indicative of a specific characteristic, trait, disease, disorder or condition.

16. The method of claim 14, wherein the at least two biomarkers are present in the same gene or in different genes.

17. (canceled)

18. The method of claim 1, wherein said control FLEXI RNAs are from one or more subjects without the specific characteristic, trait, disease, disorder or condition.

19. The method of claim 1, wherein said FLEXI RNAs comprise a panel.

20. The method of claim 19, wherein said panel further comprises control FLEXI RNAS.

21. The method of claim 19, wherein said panel further comprises other RNA or non-RNA analytes.

22. The method of claim 1, wherein said determining which differences are indicative of a specific characteristic, trait, disease, disorder or condition, is done via computer program.

23. The method of claim 1, wherein said FLEXI RNAs are specific for a cell or tissue type.

24. The method of claim 1, wherein said FLEXI RNAs are obtained from plasma.

25. The method of claim 1, wherein said FLEXI RNAs are useful in determining gene expression, alternative splicing, or differential stability.

26-99. (canceled)
